(19) United States
(12) Patent Application Publication
(10) Pub. No.: US 2024/0393689 A1
(43) Pub. Date: Nov. 28, 2024

(54) COMPOUNDS, MONOMERS, POLYMERS, PHOTORESIST COMPOSITIONS AND PATTERN FORMATION METHODS

(71) Applicant: DuPont Electronic Materials International, LLC, Marlborough, MA (US)

(72) Inventors: Li Cui, Westborough, MA (US); Emad Aqad, Northborough, MA (US); Conner Hoelzel, Watertown, MA (US); Jong Keun Park, Shrewsbury, MA (US); Choong-Bong Lee, Westborough, MA (US); Bhooshan Popere, Acton, MA (US)

(21) Appl. No.: 18/324,450

(22) Filed: May 26, 2023

(51) Int. Cl. G03F 7/039; C08F 222/20; G03F 7/004

(52) U.S. Cl. CPC G03F 7/0397; C08F 222/20; G03F 7/0045; G03F 7/0048

(57) ABSTRACT

Compounds comprise a C₆₋₃₀ aromatic or C₃₋₃₀ heteroaromatic core, wherein the core comprises a first substituent comprising (i) an enol ether group or (ii) a halomethyl ether group, wherein the halomethyl ether group is substituted or unsubstituted, a second substituent comprising an acid labile group, and a third substituent that is a halogen atom. The core is optionally further substituted. The compounds find use in the synthesis of monomers and polymers, which may be used in photoresist compositions for the manufacture of electronic devices.

### BACKGROUND OF THE INVENTION

### 1. Field of the Invention

The invention relates generally to the manufacture of electronic devices. More specifically. this invention relates to enol ether compounds, to halomethyl ether compounds, to monomers and polymers formed from such compounds, to photoresist compositions containing such polymers, and to pattern formation methods using the photoresist compositions. The compounds, monomers, polymers. photoresist compositions, and patterning methods find particular use in the formation of lithographic patterns useful in the manufacture of semiconductor devices.

### 2. Description of the Related Art

Photoresist compositions are photosensitive materials used to transfer a pattern to one or more underlying layers, such as a metal, semiconductor, or dielectric layer disposed on a substrate. Positive-tone chemically amplified photoresist compositions are conventionally used for high-resolution processing. Such resist compositions typically include a polymer having acid-labile groups and a photoacid generator (PAG). A layer of the photoresist composition is pattern-wise exposed to activating radiation and the PAG generates an acid in the exposed regions. During post-exposure baking, the acid causes cleavage of the polymer's acid-labile groups. This creates a difference in solubility characteristics between exposed and unexposed regions of the photoresist layer in a developer solution. In a positive tone development (PTD) process, exposed regions of the photoresist layer become soluble in a developer, typically an aqueous base developer, and are removed from the substrate surface. Unexposed regions, which are insoluble in the developer, remain after development to form a positive relief image. The resulting relief image permits selective processing of the substrate.

To increase the integration density of semiconductor devices and allow for the formation of structures having dimensions in the nanometer (nm) range, photoresists and photolithography processing tools having high-resolution capabilities have been and continue to be developed. One approach to achieving nm-scale feature sizes in semiconductor devices is the use of activating radiation having a short wavelength, for example, 193 nm or less, for exposure of the photoresist layer. To further improve lithographic performance, immersion lithography tools have been developed to effectively increase the numerical aperture (NA) of the lens of the imaging device. This is accomplished by use of a relatively high refractive index fluid, typically water, between the last surface of the imaging device and the upper surface of the semiconductor wafer.

ArF excimer-laser (193 nm) immersion tools are currently pushing the boundaries of lithographic processing to the 16 nm and 14 nm device nodes with the use of multiple (double, triple, or higher order) patterning techniques. The use of multiple patterning, however, can be costly in terms of increased material usage and number of process steps required as compared with single exposure, directly-imaged patterns. Photoresist compositions useful with next generation, EUV (13.5 nm) lithography have thus become of increased importance for advanced device nodes. As a result of the reduced feature sizes associated with these nodes, the performance requirements of photoresist compositions have become increasingly more stringent. Desired performance properties include, for example, high sensitivity to activating radiation, high-resolving capability, and good line-width roughness (LWR).

One way to increase EUV photoresist sensitivity is by increasing the absorption cross-section at 13.5 nm. Absorption at 13.5 nm is an atomic property of the material and can be theoretically calculated using known atomic absorptions. Typical atoms that make up resist material, such as carbon, oxygen, hydrogen, and nitrogen exhibit very low EUV absorption. Absorption cross-section can be increased, for example, by incorporation of halogen atoms (e.g., F, Cl, Br, I) into the photoresist polymer, with iodine being particularly beneficial for increasing absorption. U.S. Pat. No. 10,095,109 B1 discloses acid-cleavable iodine-containing monomers for use in forming a photoresist polymer. The monomers include a (meth)acryloyl polymerizable group and a tertiary ester acid-labile group. The acid-labile group includes a monocyclic or polycyclic unsubstituted or substituted C₆₋₃₀ arylene group or C₃₋₃₀ heteroarylene group with one or more iodine atom substituents. While incorporation of such monomer into a photoresist polymer can increase EUV absorption cross-section, the aromatic fragment that cleaves on interaction with photoacid can cause patterning defects due to insolubility in conventional TMAH developer solutions. It would therefore be desirable to have a more highly developer-soluble cleaved fragment.

There is a need in the art for compounds, monomers, polymers, photoresist compositions, and pattern formation methods which address one or more problems associated with the state of the art.

### SUMMARY OF THE INVENTION

In accordance with a first aspect of the invention, compounds are provided. The compounds comprise a C₆₋₃₀ aromatic or C₃₋₃₀ heteroaromatic core, wherein the core comprises a first substituent comprising (i) an enol ether group or (ii) a halomethyl ether group, wherein the halomethyl ether group is substituted or unsubstituted, a second substituent comprising an acid labile group, and a third substituent that is a halogen atom, wherein the core is optionally further substituted.

Also provided are monomers. The monomers comprise the reaction product of (i) an enol ether compound or a halomethyl ether compound as described herein, and (ii) a monomer precursor comprising a polymerizable group and a group chosen from an —OH group or a —C(O)OH group. The reaction product comprises an acetal or ketal linker formed by reaction between an enol ether moiety or a halomethyl ether moiety of the compound and the —OH or —C(O)OH group of the monomer precursor.

Also provided are methods of making monomers. The methods comprise reacting (i) an enol ether compound or a halomethyl ether compound as described herein, and (ii) a monomer precursor comprising a polymerizable group and a group chosen from an —OH group or a —C(O)OH group. The monomer comprises an acetal or ketal linker formed by reaction between an enol ether moiety or a halomethyl ether moiety of the compound and the —OH or —C(O)OH group of the monomer precursor. Also provided are polymers comprising a repeat unit of a monomer as described herein.

Also provided are polymers comprising the reaction product of (i) an enol ether compound or a halomethyl ether compound as described herein, and (ii) a first polymer comprising a first repeat unit comprising a group chosen from one or more of —OH or —C(O)OH. The reaction product comprises an acetal or ketal linker formed by reaction between an enol ether moiety or a halomethyl ether moiety of the compound and the —OH or —C(O)OH group of the first repeat unit, thereby forming a modified first repeat unit.

Also provided are methods of making polymers. The methods comprise reacting (i) an enol ether compound or a halomethyl ether compound as described herein, and (ii) a first polymer comprising a first repeat unit comprising a group chosen from one or more of —OH or —C(O)OH. The polymer comprises an acetal or ketal linker formed by reaction between an enol ether moiety or a halomethyl ether moiety of the compound and the —OH or —C(O)OH group of the first repeat unit, thereby forming a modified first repeat unit.

Also provided are photoresist compositions. The photoresist compositions comprise a polymer as described herein and a solvent. The photoresist compositions typically further include a photoacid generator.

Also provided are pattern formation methods. The methods comprise: (a) forming a photoresist layer from a photoresist composition as described herein on a substrate; (b) exposing the photoresist layer to activating radiation; and (c) developing the exposed photoresist layer to provide a resist relief image.

### DETAILED DESCRIPTION OF THE INVENTION

The terminology used herein is for the purpose of describing particular embodiments only and is not intended to be limiting of the invention. The singular forms “a”, “an” and “the” are intended to include singular and plural forms, unless the context indicates otherwise. All ranges disclosed herein are inclusive of the endpoints, and the endpoints are independently combinable with each other. When an element is referred to as being “on” or “over” another element, it may be directly in contact with the other element or intervening elements may be present therebetween. In contrast, when an element is referred to as being “directly on” another element, there are no intervening elements present.

Unless otherwise indicated, a group that is “substituted” refers to a group having one or more of its hydrogen atoms replaced with one or more substituents. Exemplary substituent groups include, but are not limited to, hydroxy (OH), halogen (e.g., F, Cl, I, Br), C₁₋₁₈ alkyl, C₁₋₈ haloalkyl, C₃₋₁₂ cycloalkyl, C₆₋₁₂ aryl having at least one aromatic ring (e.g., phenyl, biphenyl, naphthyl, or the like, each ring either substituted or unsubstituted aromatic), C₇₋₁₉ arylalkyl having at least one aromatic ring, C₇₋₁₂ alkylaryl, and combinations thereof. For purposes of carbon number determination, when a group is substituted, the number of carbon atoms of the group is the total number of carbon atoms in such group excluding those of any substituents.

As used herein, an “acid-labile group” refers to a group in which a bond is cleaved by the catalytic action of an acid, optionally and typically with thermal treatment, resulting in formation of a polar group, such as a carboxylic acid or alcohol group, being formed on the polymer, and optionally and typically with a moiety connected to the cleaved bond becoming disconnected from the polymer. Such acid is typically a photo-generated acid with bond cleavage occurring during post-exposure baking. Suitable acid-labile groups include, for example: tertiary alkyl ester groups, secondary or tertiary aryl ester groups, secondary or tertiary ester groups having a combination of alkyl and aryl groups, tertiary alkoxy groups, acetal groups, or ketal groups. Acid-labile groups are also referred to in the art as “acid-cleavable groups,” “acid-cleavable protecting groups,” “acid-labile protecting groups,” “acid-leaving groups,” “acid-decomposable groups,” and “acid-sensitive groups.”

### Enol Ether Compounds

Enol ether compounds of the present invention comprise a C₆₋₃₀ aromatic or C₃₋₃₀ heteroaromatic core, wherein the core comprises a first substituent comprising an enol ether group, a second substituent comprising an acid labile group, and a third substituent that is a halogen atom, for example, F, Cl, Br, or I, with F and I being preferred, and I being particularly preferred. The core may be further substituted or further unsubstituted.

Suitable enol ether compounds of the invention include, for example, those represented by formula (1):

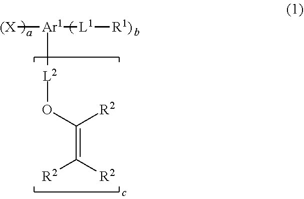

wherein: Ar¹ is a C₆₋₃₀ aromatic or C₃₋₃₀ heteroaromatic core; R¹ independently represents a moiety comprising an acid labile group; each R² independently represents H, substituted or unsubstituted C₁₋₁₀ alkyl, substituted or unsubstituted C₁₋₁₀ haloalkyl, optionally including as part of its structure one or more groups chosen from —O—, —S—, —N(R³)—, —C(O)—, —C(O)O—, or —C(O)N(R³)—, wherein R³ represents H, substituted or unsubstituted C₁₋₁₀ alkyl, substituted or unsubstituted C₁₋₁₀ heteroalkyl, substituted or unsubstituted C₆₋₃₀ aryl, or substituted or unsubstituted C₄₋₃₀ heteroaryl, any two R² groups together optionally forming a ring, and one or more R² groups and L¹ together optionally forming a ring; X independently represents a halogen atom; each L¹ and L² independently represents a single bond or a linking group; and a, b, and c independently represent an integer greater than or equal to 1;
wherein the core is optionally further substituted. As used herein “optionally further substituted” means optionally including one or more additional substituents beyond the substituents shown in the applicable general formula.

It is preferred that Ar¹ is chosen from pyridine, benzene, naphthalene, acenaphthylene, quinoline, isoquinoline, fluorene, carbazole, anthracene, phenanthrene, pyrene, coronene, triphenylene, chrysene, phenalene, benz[a]anthracene, dibenz[a,h]anthracene, benzo[a]pyrene, or pentacene, and more preferably from benzene, naphthalene, anthracene, phenanthrene, pyrene, coronene, triphenylene, chrysene, phenalene, benz[a]anthracene, dibenz[a,h]anthracene, and benzo[a]pyrene, and even more preferably from benzene, naphthalene, anthracene, phenanthrene, pyrene, coronene, triphenylene, chrysene, and phenalene, and most preferably Ar¹ is benzyl or naphthyl, each of which may be optionally further substituted.

Suitable acid-labile groups for R¹ include, for example, one or more of acid-labile ester groups including tertiary ester groups, such as tertiary alkyl esters, tertiary aryl esters, and tertiary esters having a combination of aryl and alkyl groups bonded to the tertiary carbon atom, and secondary esters, such as secondary aryl esters and secondary esters having an aryl group and an alkyl group bonded to the secondary carbon atoms, tertiary alkoxy groups, acetal groups, and ketal groups. Preferably, R¹ comprises an acid-labile ester group, an acetal group, or a ketal group.

Suitable exemplary acid-labile groups include the following:

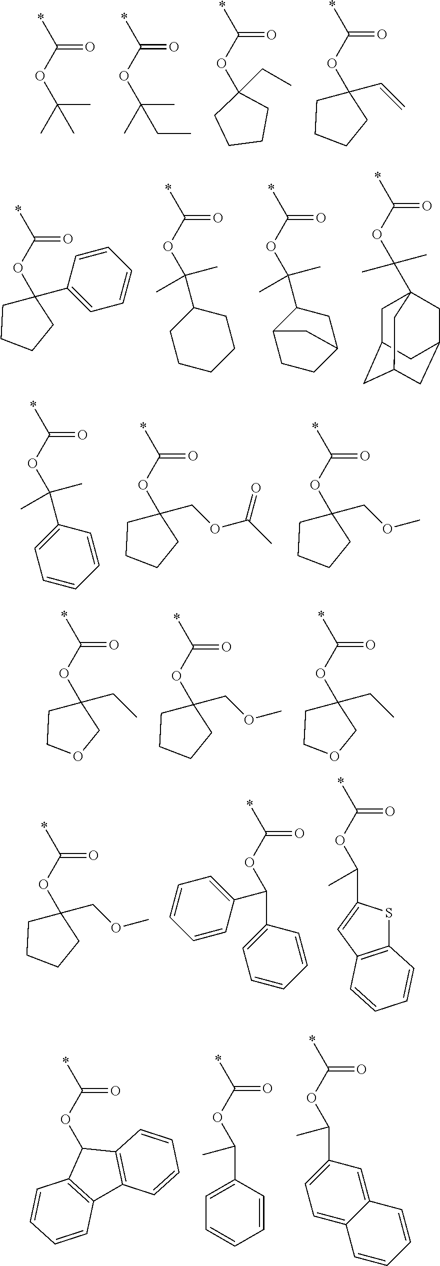

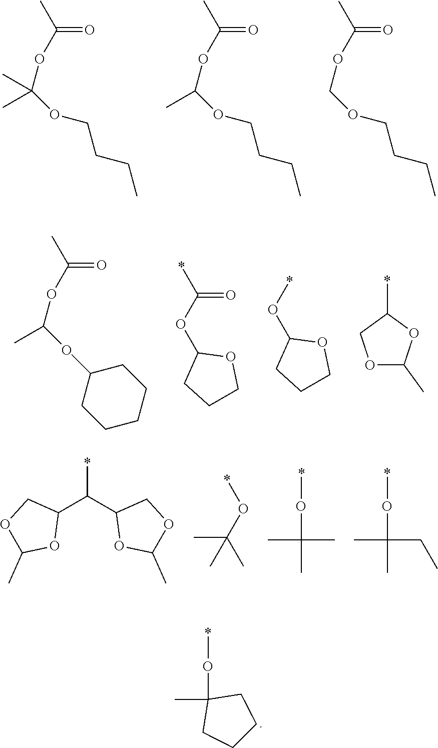

wherein * denotes a point of attachment to another portion of the compound.

Preferable for R² are H, substituted or unsubstituted C₁₋₁₀ alkyl, or substituted or unsubstituted C₁₋₁₀ fluoroalkyl, and any two R² groups together optionally forming a ring.

Preferable halogen atoms for X are F, Cl, Br, and I. Of these, fluorine and iodine atoms are more preferred, with iodine being particularly preferred.

L¹ and L² are typically a single bond or a linking group comprising one or more of: substituted or unsubstituted C₁₋₃₀ alkylene, substituted or unsubstituted C₁₋₃₀ heteroalkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted C₇₋₃₀ arylalkylene, substituted or unsubstituted C₁₋₃₀ heteroarylene, substituted or unsubstituted C₃₋₃₀ heteroarylalkylene, —O—, —C(O)—, —C(O)—O—, —S—, —S(O)₂—, or —N (R⁴⁴)—S(O)₂—, wherein R⁴⁴ is H, a straight chain or branched C₁₋₂₀ alkyl, monocyclic or polycyclic C₃₋₂₀ cycloalkyl, or monocyclic or polycyclic C₃₋₂₀ heterocycloalkyl.

a and b are each typically an integer from 1 to 6, and more typically from 1 to 3, or from 1 to 2. a+b can be an integer from 2 to the total number of available (not otherwise substituted) aromatic ring carbon atoms of Ar¹. a+b is typically an integer from 2 to 10, more typically from 2 to 6, or from 2 to 4. c is typically an integer from 1 to 3, and more typically 1 or 2.

Suitable enol ether compounds of formula (1) include, for example, the following:

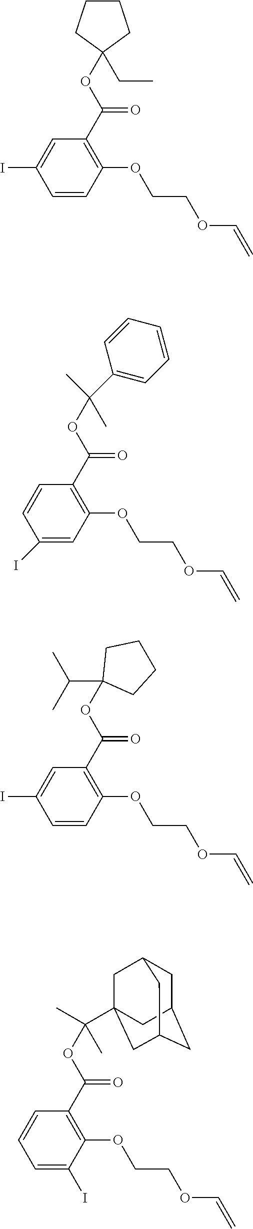 [SMILES: C=COCCOc1ccc(I)cc1C(=O)OC1(CC)CCCC1 ; C=COCCOc1cc(I)ccc1C(=O)OC(C)(C)c1ccccc1 ; C=COCCOc1ccc(I)cc1C(=O)OC1(C(C)C)CCCC1 ; C=COCCOc1c(I)cccc1C(=O)OC(C)(C)C12CC3CC(CC(C3)C1)C2]

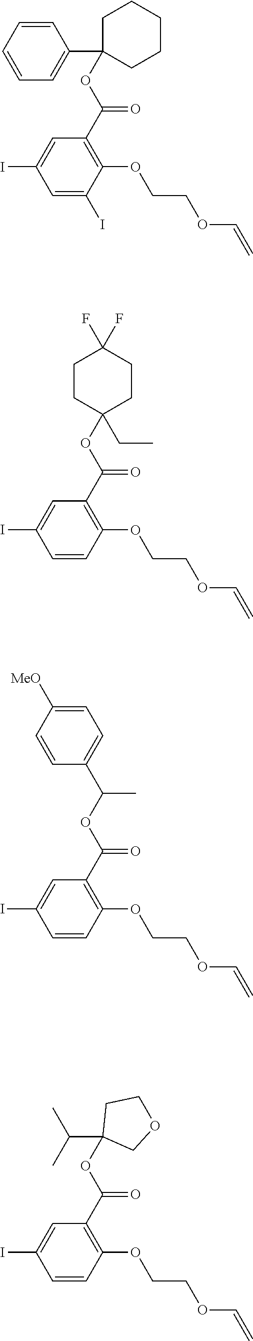 [SMILES: C=COCCOc1c(I)cc(I)cc1C(=O)OC1(c2ccccc2)CCCCC1 ; C=COCCOc1ccc(I)cc1C(=O)OC1(CC)CCC(F)(F)CC1 ; C=COCCOc1ccc(I)cc1C(=O)OC(C)c1ccc(OC)cc1 ; C=COCCOc1ccc(I)cc1C(=O)OC1(C(C)C)CCOC1]

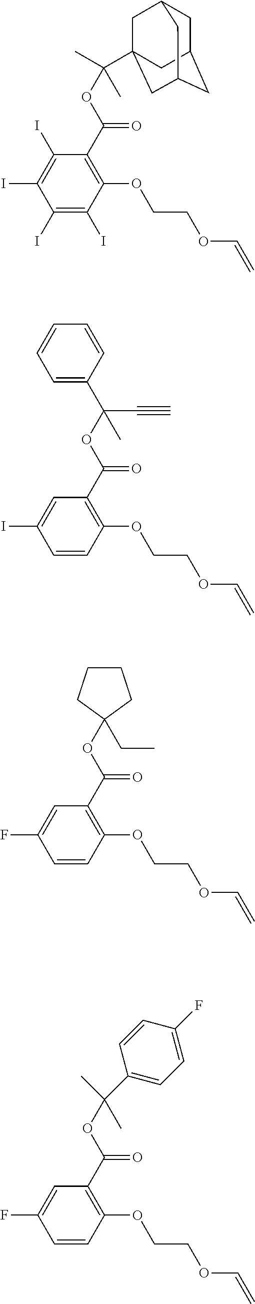 [SMILES: C=COCCOc1c(I)c(I)c(I)c(I)c1C(=O)OC(C)(C)C12CC3CC(CC(C3)C1)C2 ; C#CC(C)(OC(=O)c1cc(I)ccc1OCCOC=C)c1ccccc1 ; C=COCCOc1ccc(F)cc1C(=O)OC1(CC)CCCC1 ; C=COCCOc1ccc(F)cc1C(=O)OC(C)(C)c1ccc(F)cc1]

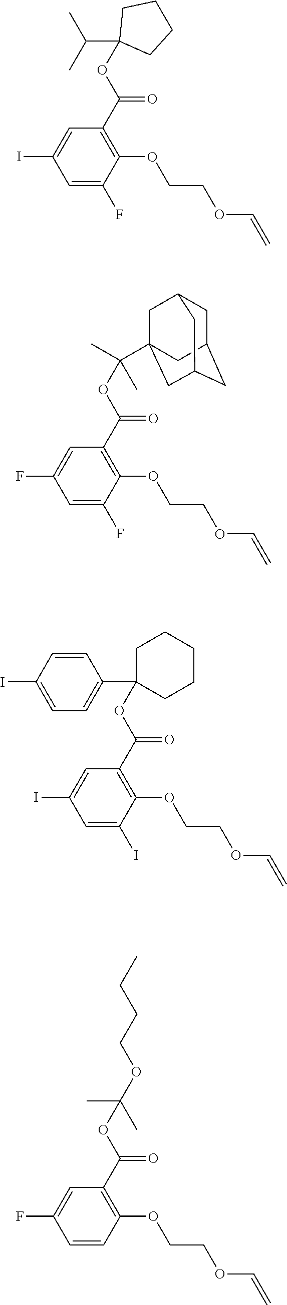 [SMILES: C=COCCOc1c(F)cc(I)cc1C(=O)OC1(C(C)C)CCCC1 ; C=COCCOc1c(F)cc(F)cc1C(=O)OC(C)(C)C12CC3CC(CC(C3)C1)C2 ; C=COCCOc1c(I)cc(I)cc1C(=O)OC1(c2ccc(I)cc2)CCCCC1 ; C=COCCOc1ccc(F)cc1C(=O)OC(C)(C)OCCCC]

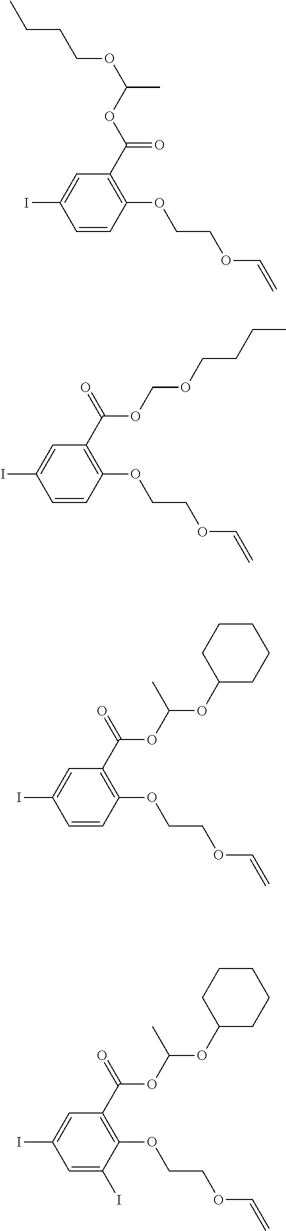

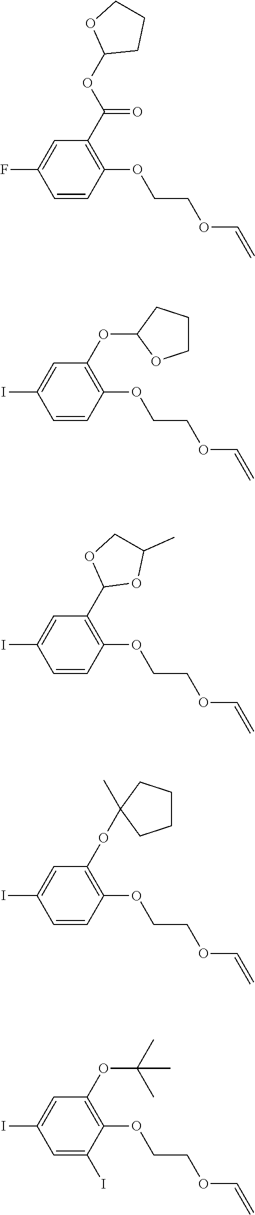 [SMILES: C=COCCOc1ccc(F)cc1C(=O)OC1CCCO1 ; C=COCCOc1ccc(I)cc1OC1CCCO1 ; C=COCCOc1ccc(I)cc1C1OCC(C)O1 ; C=COCCOc1ccc(I)cc1OC1(C)CCCC1 ; C=COCCOc1c(I)cc(I)cc1OC(C)(C)C]

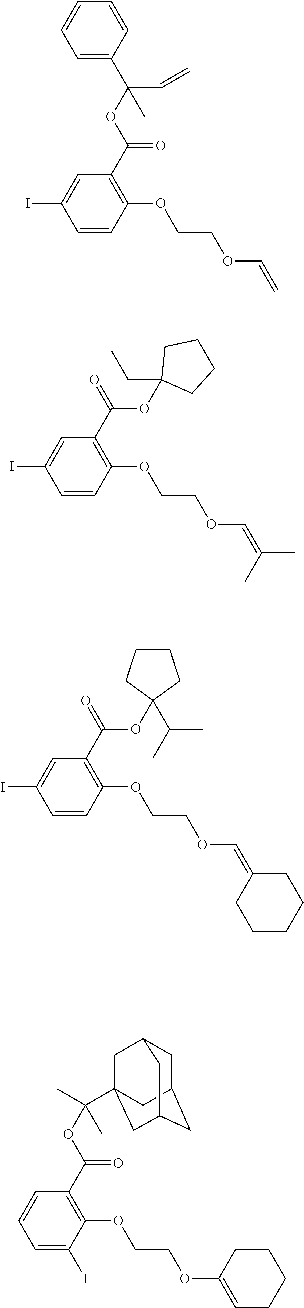 [SMILES: C=COCCOc1ccc(I)cc1C(=O)OC(C)(C=C)c1ccccc1 ; CCC1(OC(=O)c2cc(I)ccc2OCCOC=C(C)C)CCCC1 ; CC(C)C1(OC(=O)c2cc(I)ccc2OCCOC=C2CCCCC2)CCCC1 ; CC(C)(OC(=O)c1cccc(I)c1OCCOC1=CCCCC1)C12CC3CC(CC(C3)C1)C2]

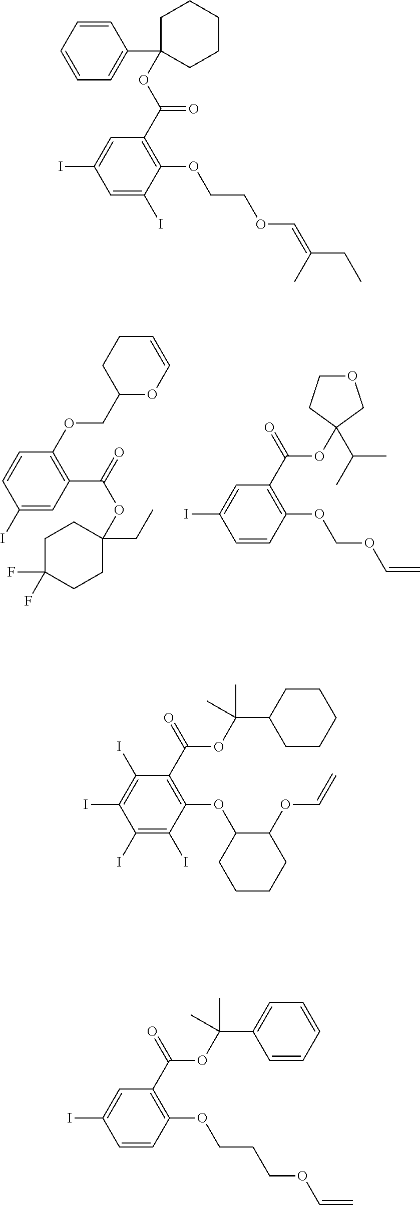

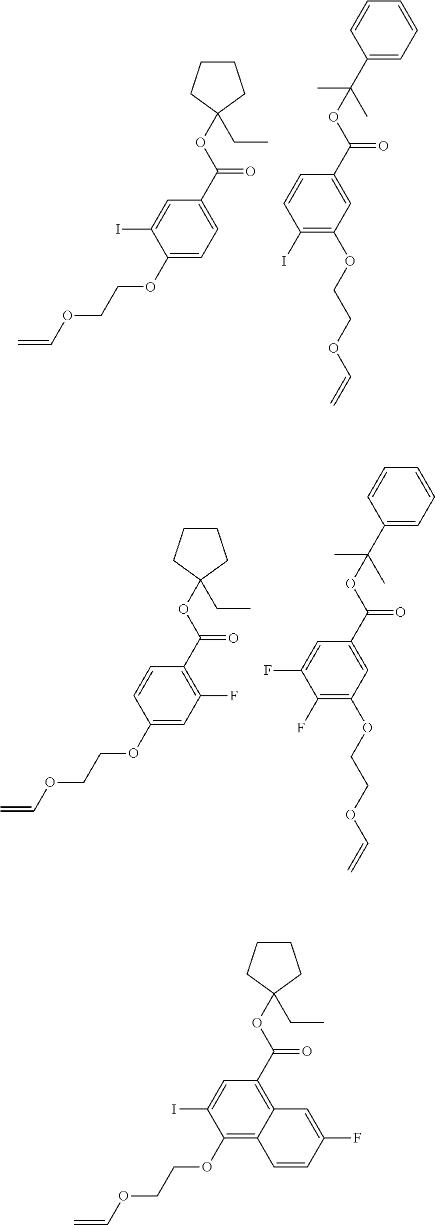

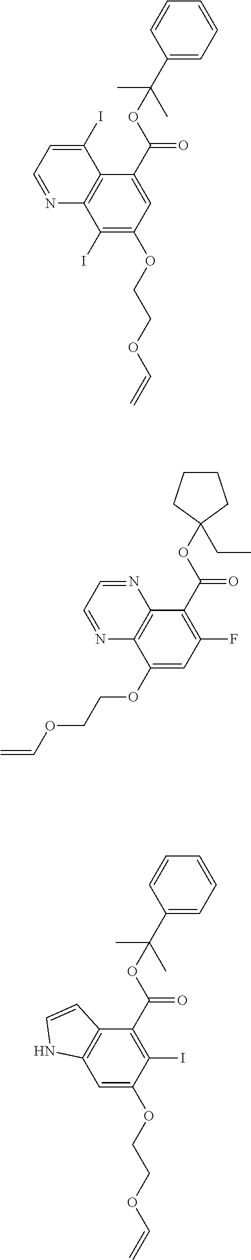 [SMILES: C=COCCOc1cc(C(=O)OC(C)(C)c2ccccc2)c2c(I)ccnc2c1I ; C=COCCOc1cc(F)c(C(=O)OC2(CC)CCCC2)c2nccnc12 ; C=COCCOc1cc2[nH]ccc2c(C(=O)OC(C)(C)c2ccccc2)c1I]

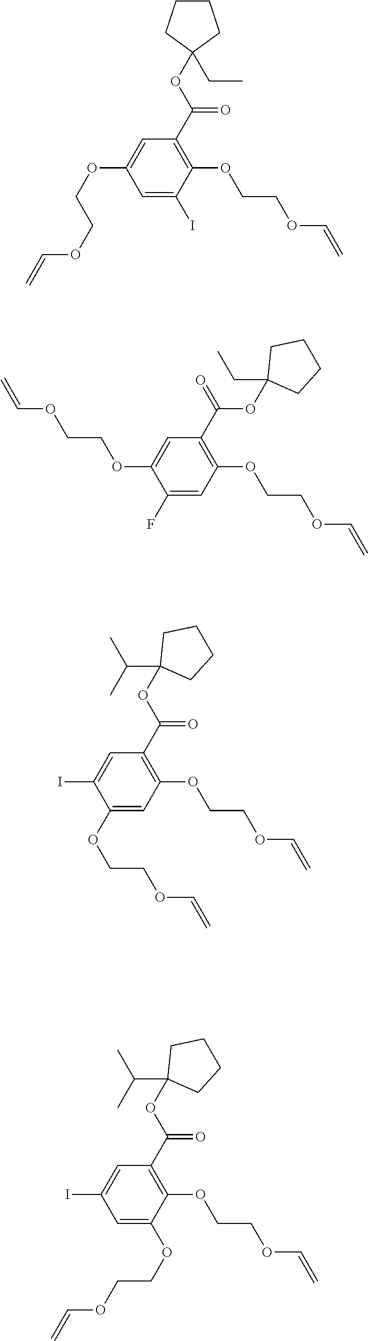

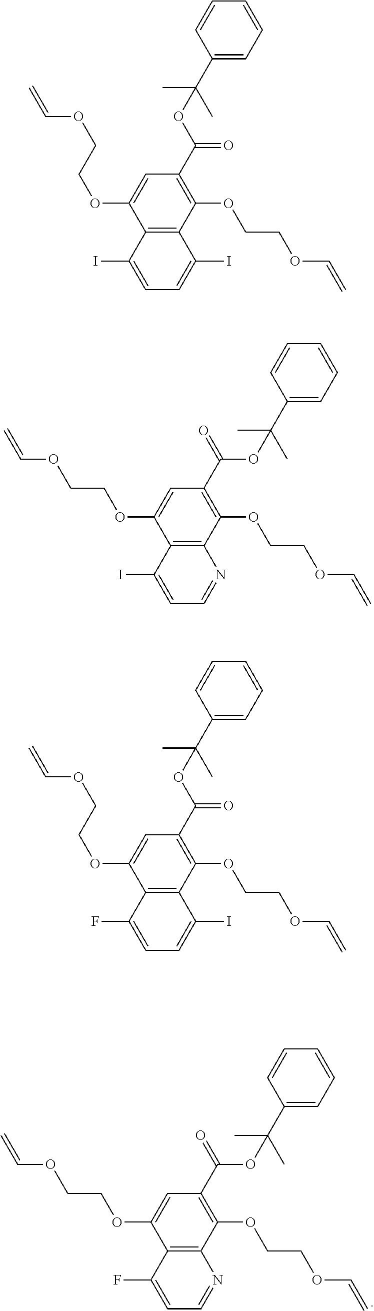

Enol ether compounds as described above can readily be made by persons skilled in the art using known synthesis techniques. For example, the compounds may be prepared by mixing reactants, for example, a phenol with vinyloxy alkyl chloride, bromide or iodide, base and solvent, optionally with an alkali halide catalyst, preferably an alkali iodide. The solvent is typically an organic solvent, for example, N,N-dimethylformamide, tetrahydrofuran, dimethyl sulfoxide, N-methyl-2-pyrrolidone, 1,4-dioxane, or combinations thereof. The reaction is carried at a temperature and time effective to cause reaction of the reactants in the reaction mixture to form the enol ether. The reaction temperature is typically from 0 to 150° C., preferably from 25 to 100° C. The reaction time is typically from 1 to 96 hours, preferably from 2 to 24 hours. The product compounds can be purified by techniques known in the art such as column chromatography.

Enol ether compounds as described herein find particular use in the formation of acid-sensitive monomers and acid-sensitive polymers. In a first aspect, the enol ether compounds may be used as a direct reactant to form the acid-sensitive monomer, which acid-sensitive monomer may be polymerized to form an acid-sensitive polymer. In a further aspect, the enol ether compound may be used as a direct reactant to form the acid-sensitive polymer by reaction with a precursor (or base) polymer. Acid-sensitive monomers and polymers of the invention are described below.

### Halomethyl Ether Compounds

Suitable halomethyl ether compounds of the invention include, for example, those represented by formula (2):

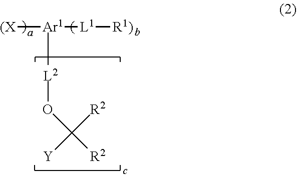

wherein: Ar¹, R¹, R², X, L¹, L², a, b, and c are as defined above with reference to formula (1); and Y independently represents Cl, Br, or I. The core is optionally further substituted.

Suitable halomethyl ether compounds of formula (2) include, for example, the following:

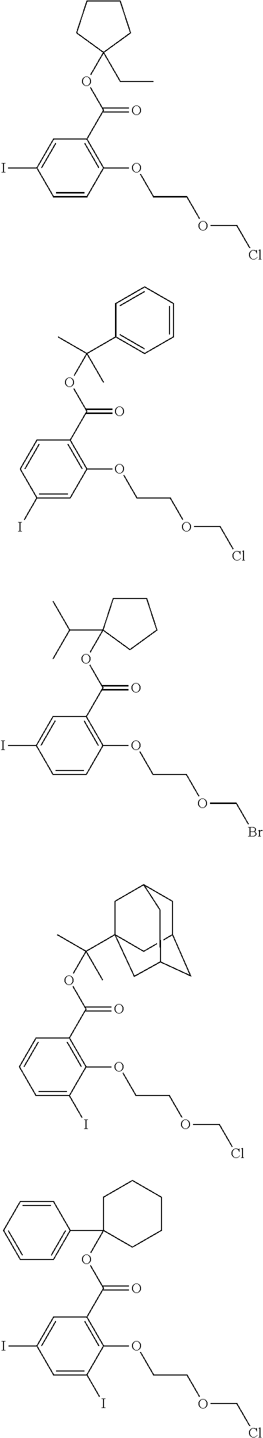 [SMILES: CCC1(OC(=O)c2cc(I)ccc2OCCOCCl)CCCC1 ; CC(C)(OC(=O)c1ccc(I)cc1OCCOCCl)c1ccccc1 ; CC(C)C1(OC(=O)c2cc(I)ccc2OCCOCBr)CCCC1 ; CC(C)(OC(=O)c1cccc(I)c1OCCOCCl)C12CC3CC(CC(C3)C1)C2 ; O=C(OC1(c2ccccc2)CCCCC1)c1cc(I)cc(I)c1OCCOCCl]

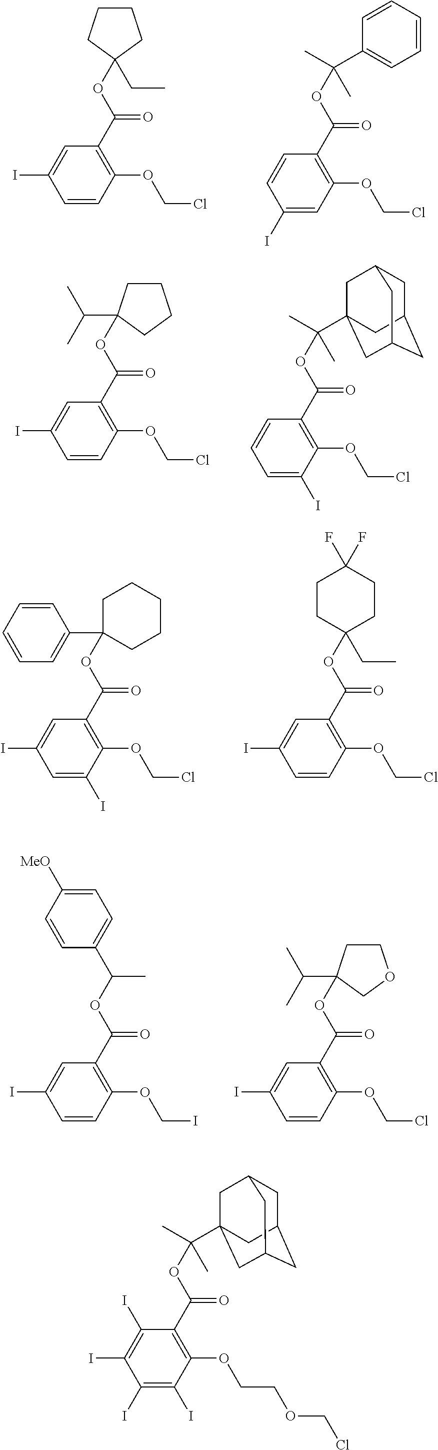

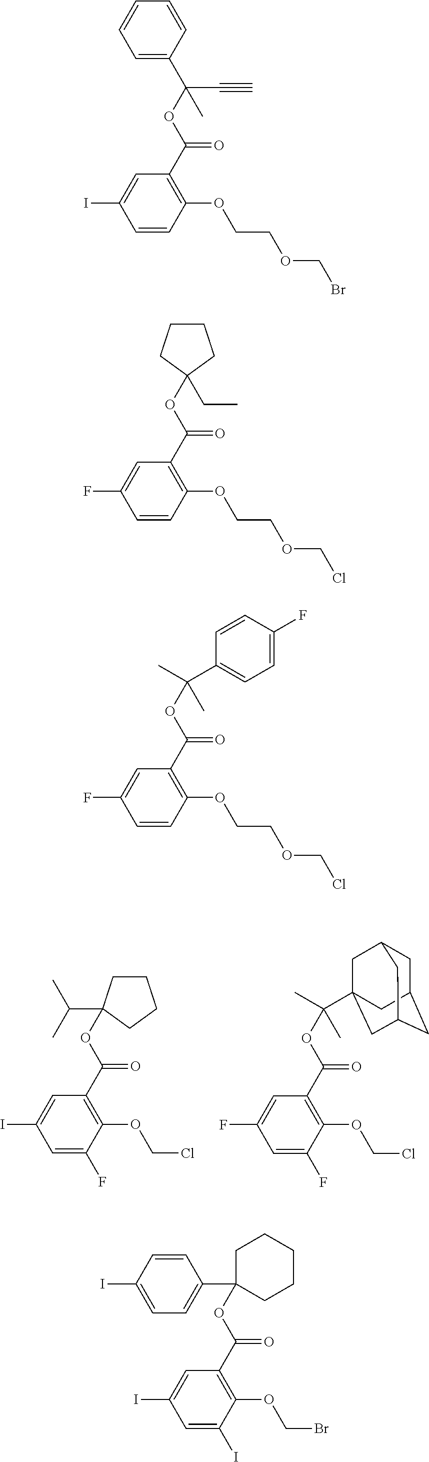 [SMILES: C#CC(C)(OC(=O)c1cc(I)ccc1OCCOCBr)c1ccccc1 ; CCC1(OC(=O)c2cc(F)ccc2OCCOCCl)CCCC1 ; CC(C)(OC(=O)c1cc(F)ccc1OCCOCCl)c1ccc(F)cc1 ; CC(C)C1(OC(=O)c2cc(I)cc(F)c2OCCl)CCCC1 ; CC(C)(OC(=O)c1cc(F)cc(F)c1OCCl)C12CC3CC(CC(C3)C1)C2 ; O=C(OC1(c2ccc(I)cc2)CCCCC1)c1cc(I)cc(I)c1OCBr]

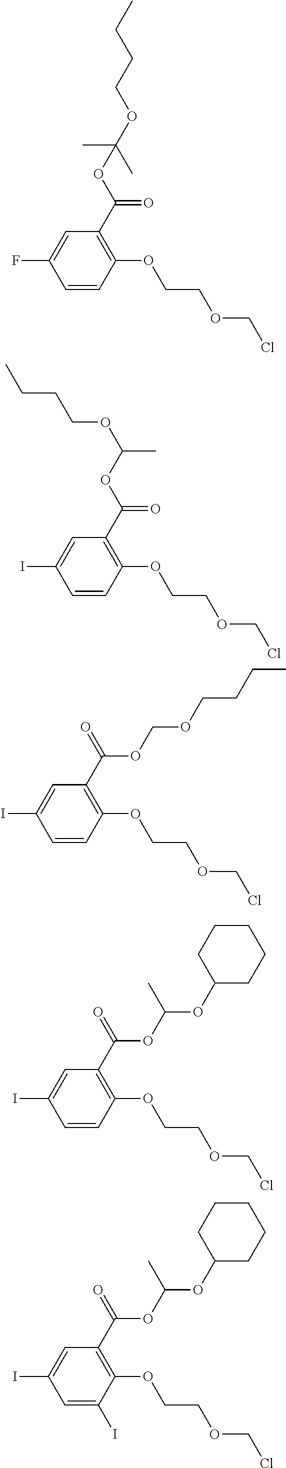

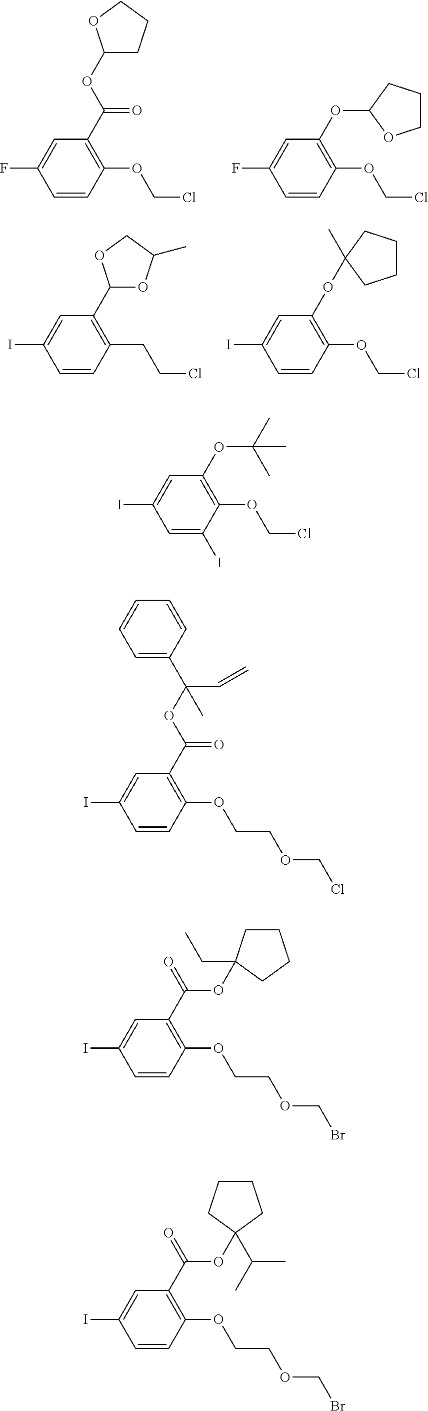

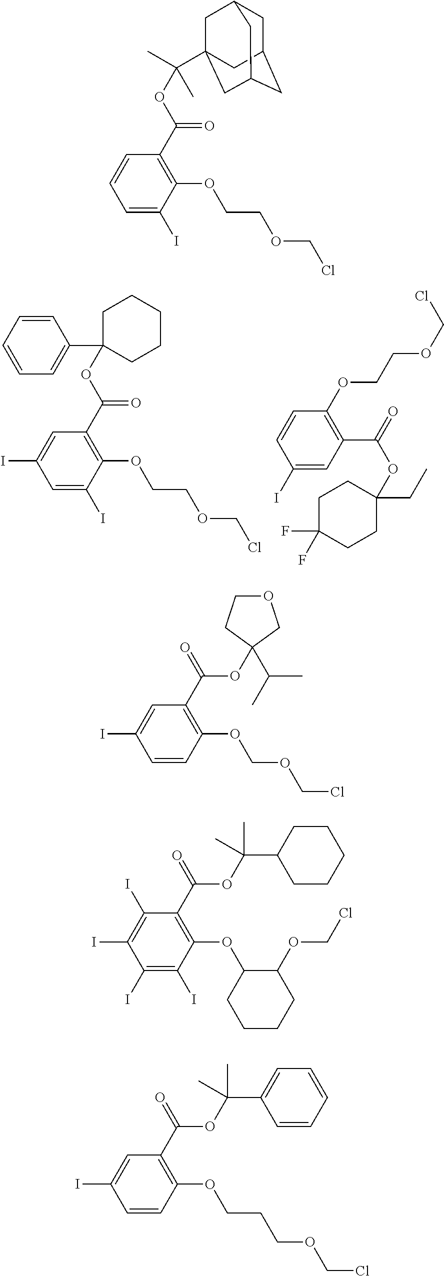

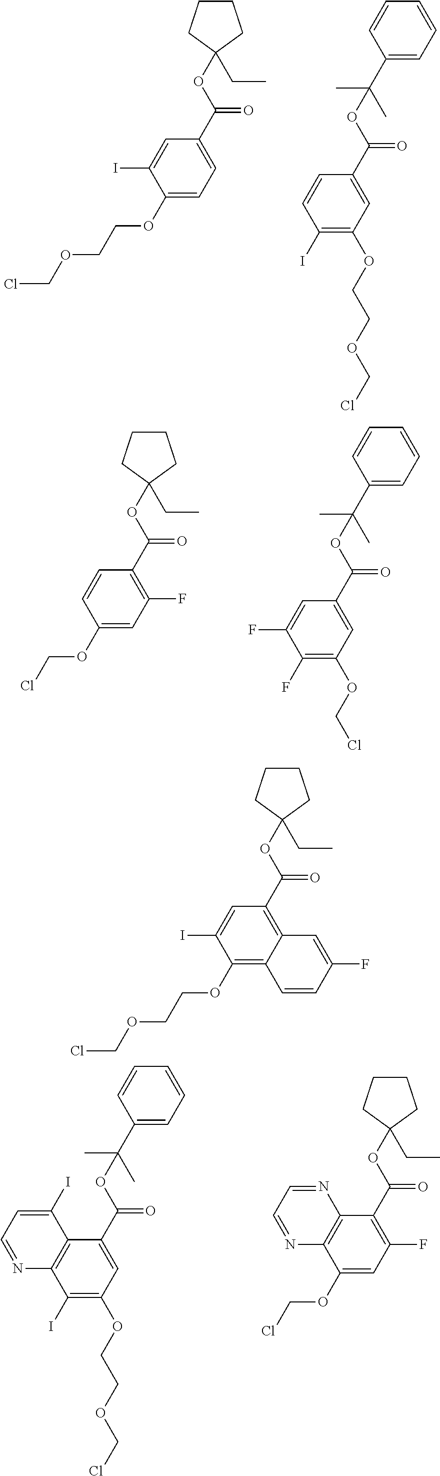

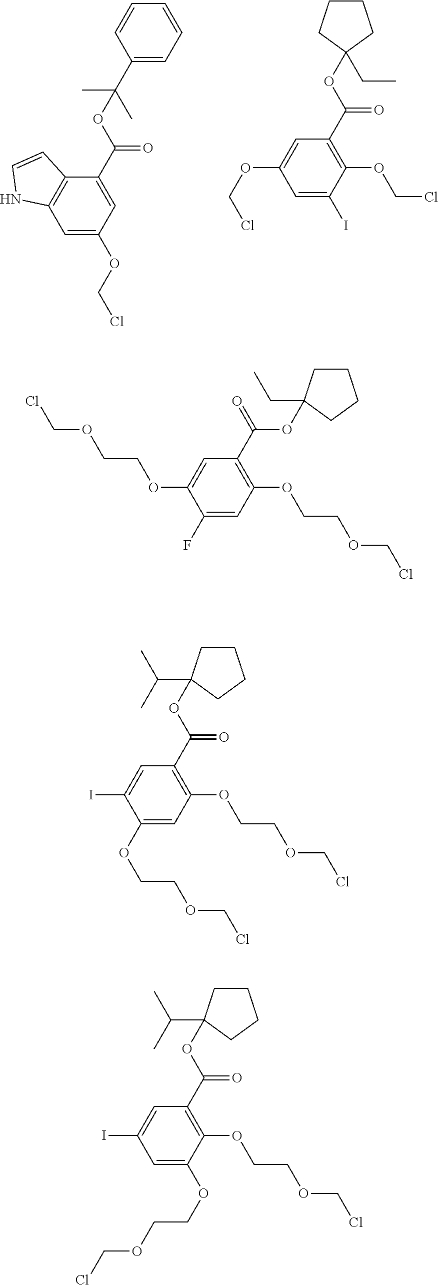 [SMILES: CC(C)(OC(=O)c1cc(OCCl)cc2[nH]ccc12)c1ccccc1 ; CCC1(OC(=O)c2cc(OCCl)cc(I)c2OCCl)CCCC1 ; CCC1(OC(=O)c2cc(OCCOCCl)c(F)cc2OCCOCCl)CCCC1 ; CC(C)C1(OC(=O)c2cc(I)c(OCCOCCl)cc2OCCOCCl)CCCC1 ; CC(C)C1(OC(=O)c2cc(I)cc(OCCOCCl)c2OCCOCCl)CCCC1]

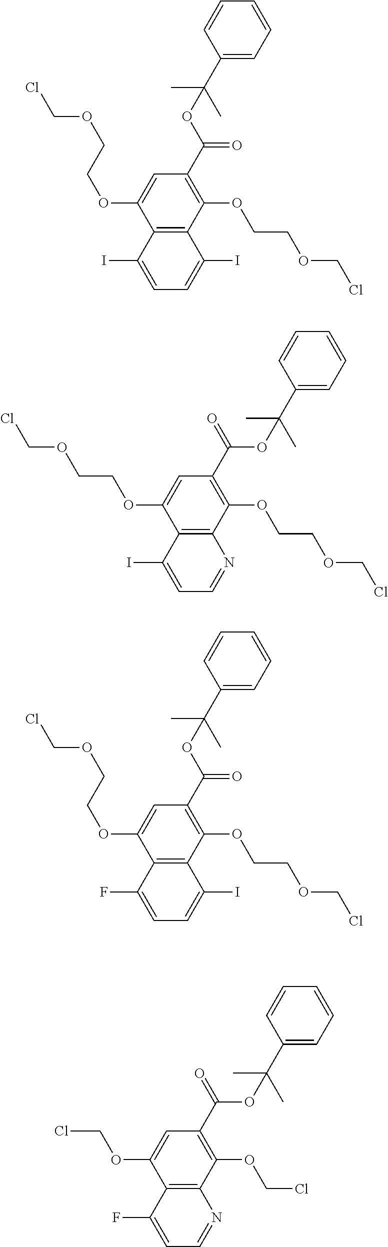

Halomethyl ether compounds of the invention can readily be made by persons skilled in the art using known synthesis techniques. For example, the halomethyl ether group can be covalently connected to a substrate having an alkyl alcohol group or an aryl alcohol group by reacting the OH group with an aqueous solution of formaldehyde or paraformaldehyde, and subsequent treatment with an aqueous solution of hydrogen chloride or hydrogen bromide. An exemplary process for the addition of a halomethyl ether group to a substrate having an alkyl alcohol group, and subsequent esterification reaction with a tertiary ester to from a halomethyl ether compound in accordance with the invention is illustrated in the following scheme:

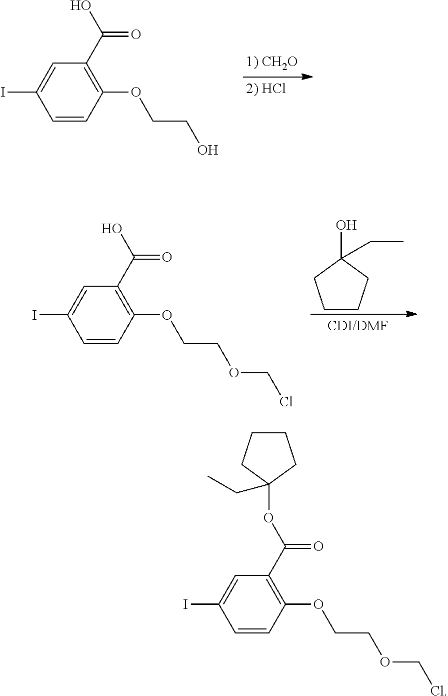

Halomethyl ether compounds as described herein find particular use in the formation of acid-sensitive monomers and acid-sensitive polymers. In a first aspect, the halomethyl ether compounds may be used as a reactant to form the acid-sensitive monomer, which acid-sensitive monomer may be polymerized to form an acid-sensitive polymer. In a further aspect, the halomethyl ether compound may be used as a direct reactant to form the acid-sensitive polymer by reaction with a precursor (or base) polymer. Acid-sensitive monomers and polymers of the invention are described below.

### Acid-Sensitive Monomers

The enol ether compounds or the halomethyl ether compounds described herein can be used in the synthesis of acid-sensitive monomers that may be used in the synthesis of polymers. The acid-sensitive monomers comprise the reaction product of (i) an enol ether compound or a halomethyl ether compound as described herein, and (ii) a monomer precursor comprising a polymerizable group, typically a group comprising an ethylenically unsaturated carbon-carbon double bond, for example, a polymerizable vinyl group, and a group chosen from an —OH group or a —C(O)OH group. The reaction product comprises an acetal or ketal linker formed by reaction between an enol ether moiety of the enol ether compound or a halomethyl ether moiety of the halomethyl ether compound and the —OH or —C(O)OH group of the monomer precursor.

Suitable polymerizable groups for the monomer precursor (and resulting acid-sensitive monomer) include an ethylenically unsaturated carbon-carbon double bond. The polymerizable groups is typically chosen from a substituted or unsubstituted C₂₋₂₀ alkenyl group, a substituted or unsubstituted norbornyl group, a substituted or unsubstituted (meth)acrylic group, a substituted or unsubstituted vinyl ether group, a substituted or unsubstituted vinyl ketone group, a substituted or unsubstituted vinyl ester group, a substituted or unsubstituted vinyl aromatic group, or a substituted or unsubstituted N-maleimide group. Of these, a substituted or unsubstituted (meth)acrylic group and a substituted or unsubstituted vinyl aromatic group are preferred.

Suitable monomer precursors are not particularly limited and include, for example, the following:

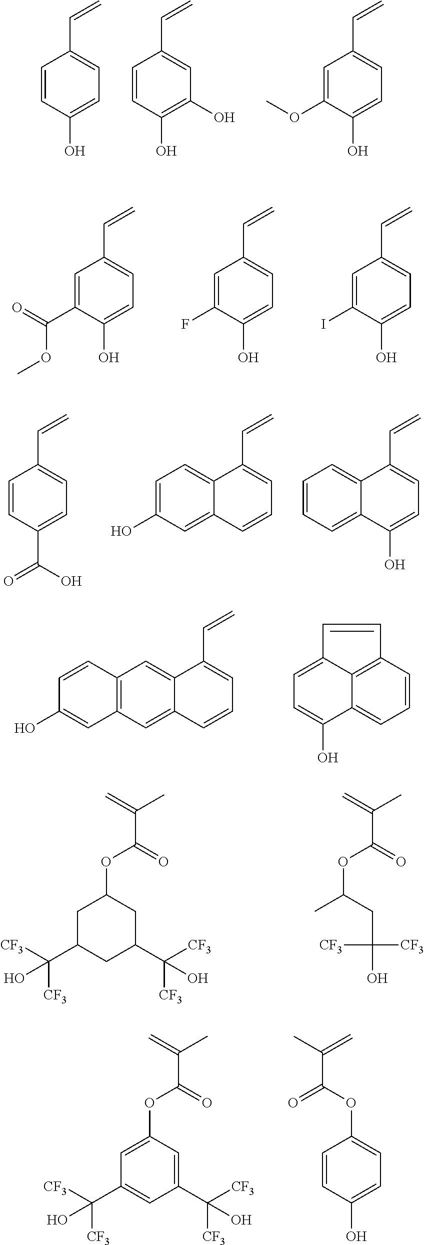

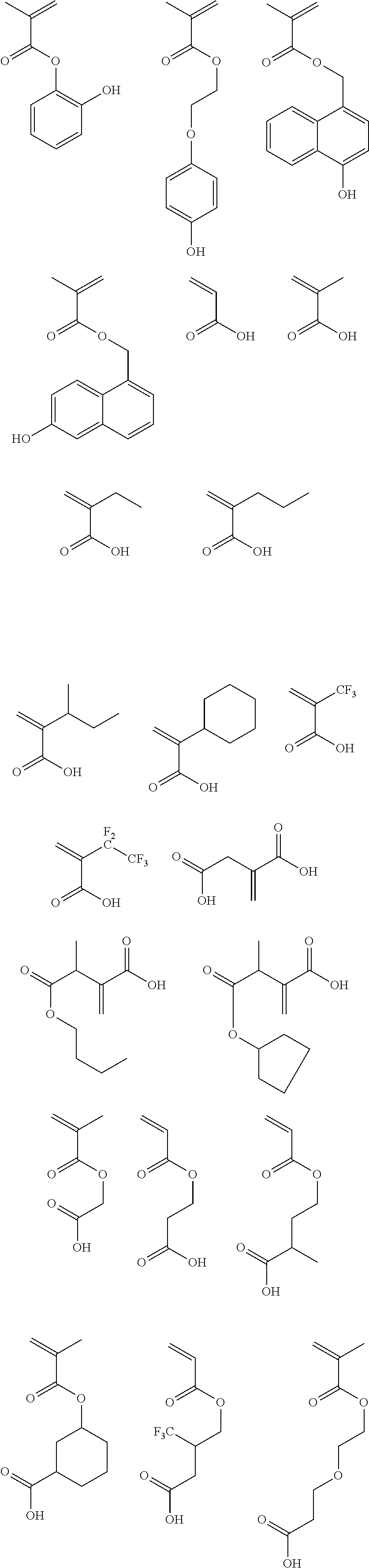

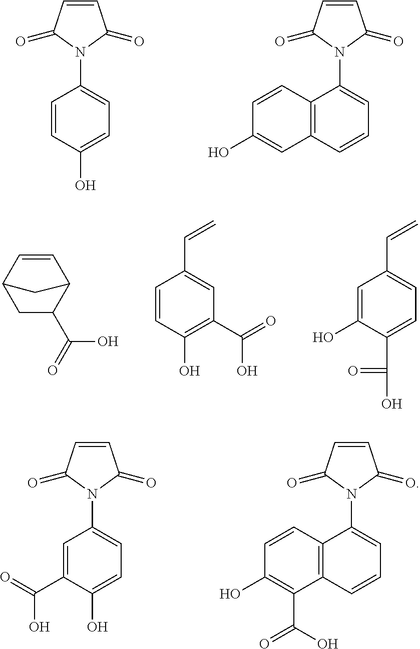

Suitable monomer precursors are commercially available or can be made using methods known in the art.

The acid-sensitive monomer preferably comprises a moiety of formula (3):

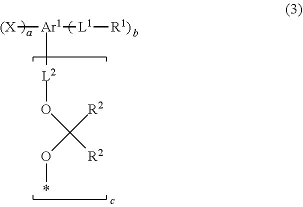

wherein: Ar¹, R¹, X, L¹, L², a, b, and c are as described above with reference to formula (1); R⁴ independently represents H, substituted or unsubstituted C₁₋₁₀ alkyl, substituted or unsubstituted C₁₋₁₀ heteroalkyl, substituted or unsubstituted C₆₋₃₀ aryl, or substituted or unsubstituted C₄₋₃₀ heteroaryl, any two R⁴ groups together optionally forming a ring, an R⁴ group and L² together optionally forming a ring, and an R⁴ group and another portion of the monomer not shown in formula (3) together optionally forming a ring; and * represents an attachment point to another portion of the monomer. Preferable for R⁴ are H, substituted or unsubstituted C₁₋₅ alkyl, substituted or unsubstituted C₁₋₅ fluoroalkyl, or substituted or unsubstituted C₁₋₅ heteroalkyl.

The acid-sensitive monomer preferably is of the following formula (4):

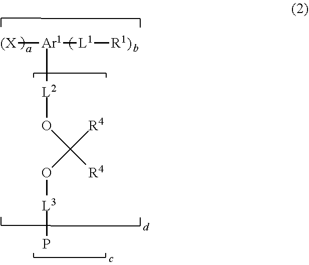

wherein: Ar¹, R¹, R⁴, X, L¹, L², a, b, and c are as described above with reference to formula (3); L³ is independently a single bond or a divalent linking group; P represents a polymerizable group as described above with reference to the monomer precursor; and d is 1 or 2, provided when c is greater than 1, d is 1.

L³ is typically a single bond or a divalent linking group comprising one or more of: substituted or unsubstituted C₁₋₃₀ alkylene, substituted or unsubstituted C₁₋₃₀ heteroalkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted C₇₋₃₀ arylalkylene, substituted or unsubstituted C₁₋₃₀ heteroarylene, substituted or unsubstituted C₃₋₃₀ heteroarylalkylene, —O—, —C(O)—, —C(O)—O—, —S—, —S(O)₂—, or —N(R⁴⁴)—S(O)₂—, wherein R⁴⁴ is H, a straight chain or branched C₁₋₂₀ alkyl, monocyclic or polycyclic C₃₋₂₀ cycloalkyl, or monocyclic or polycyclic C₃₋₂₀ heterocycloalkyl. The L³ divalent linking group preferably comprises an aromatic group covalently bonded to the adjacent acetal or ketal oxygen atom.

Preferable for P are polymerizable groups with a vinyl group, such as vinyl aromatic (e.g., styrene) and (meth)acrylate groups.

The acid-sensitive monomer is typically of the following formula (4a) or (4b):

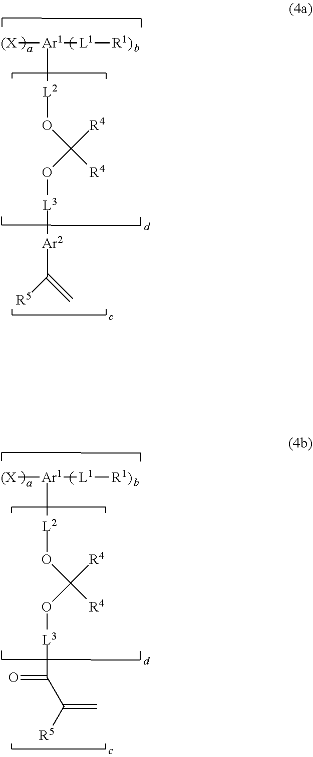

wherein: Ar¹, R¹, R⁴, X, L¹, L², L³, a, b, c, and d are as described above with reference to formula (4); R⁵ independently represents H, a halogen atom, or substituted or unsubstituted C₁₋₆ alkyl, with H or methyl being typical; and Ar² is a substituted or unsubstituted C₆₋₃₀ aromatic group or a substituted or unsubstituted C₃₋₃₀ heteroaromatic group. Ar² may be monocyclic or polycyclic and may be chosen, for example, from polyvalent forms of benzene, pyridine, naphthalene, acenaphthylene, quinoline, isoquinoline, fluorene, carbazole, anthracene, phenanthrene, pyrene, coronene, triphenylene, chrysene, phenalene, benz[a]anthracene, dibenz[a,h]anthracene, benzo[a]pyrene, or pentacene, preferably from polyvalent forms of benzene, naphthalene, anthracene, phenanthrene, pyrene, coronene, triphenylene, chrysene, phenalene, benz[a]anthracene, dibenz[a,h]anthracene, and benzo[a]pyrene, more preferably from polyvalent forms of benzene, naphthalene, anthracene, phenanthrene, pyrene, coronene, triphenylene, chrysene, and phenalene, and most preferably from polyvalent forms of benzene (e.g., phenylene).

Non-limiting examples of the acid-sensitive monomer include the following:

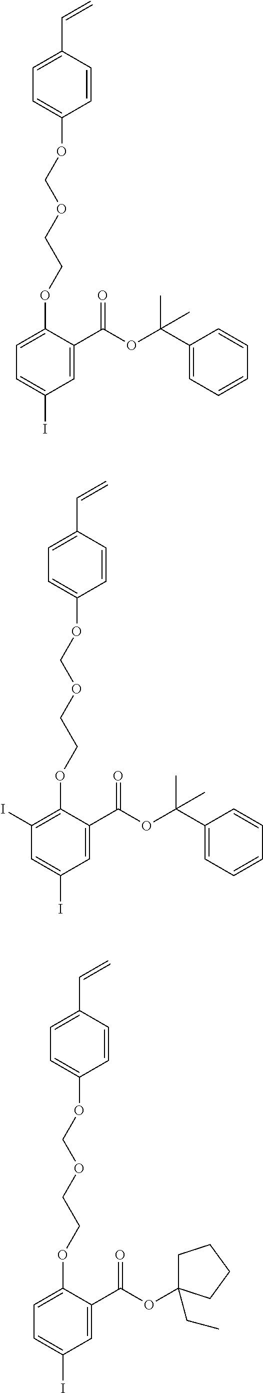 [SMILES: C=Cc1ccc(OCOCCOc2ccc(I)cc2C(=O)OC(C)(C)c2ccccc2)cc1 ; C=Cc1ccc(OCOCCOc2c(I)cc(I)cc2C(=O)OC(C)(C)c2ccccc2)cc1 ; C=Cc1ccc(OCOCCOc2ccc(I)cc2C(=O)OC2(CC)CCCC2)cc1]

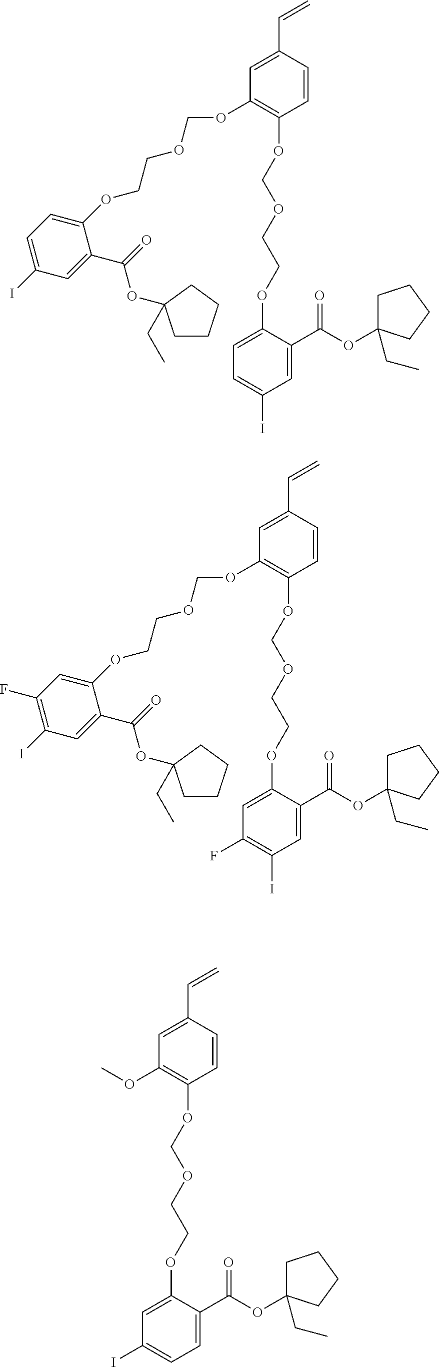 [SMILES: C=Cc1ccc(OCOCCOc2ccc(I)cc2C(=O)OC2(CC)CCCC2)c(OCOCCOc2ccc(I)cc2C(=O)OC2(CC)CCCC2)c1 ; C=Cc1ccc(OCOCCOc2cc(F)c(I)cc2C(=O)OC2(CC)CCCC2)c(OCOCCOc2cc(F)c(I)cc2C(=O)OC2(CC)CCCC2)c1 ; C=Cc1ccc(OCOCCOc2cc(I)ccc2C(=O)OC2(CC)CCCC2)c(OC)c1]

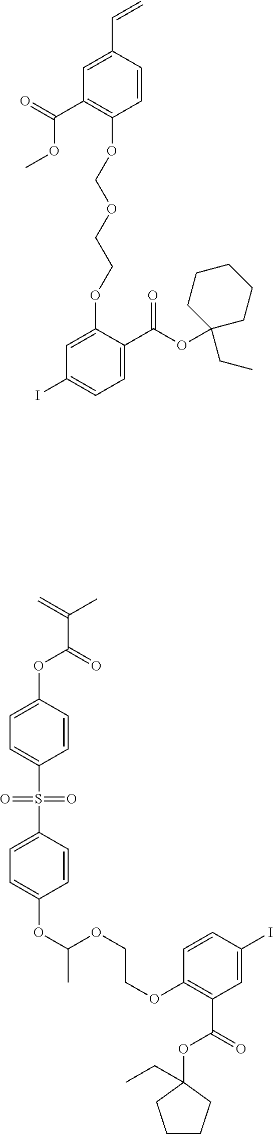 [SMILES: C=Cc1ccc(OCOCCOc2cc(I)ccc2C(=O)OC2(CC)CCCCC2)c(C(=O)OC)c1 ; C=C(C)C(=O)Oc1ccc(S(=O)(=O)c2ccc(OC(C)OCCOc3ccc(I)cc3C(=O)OC3(CC)CCCC3)cc2)cc1]

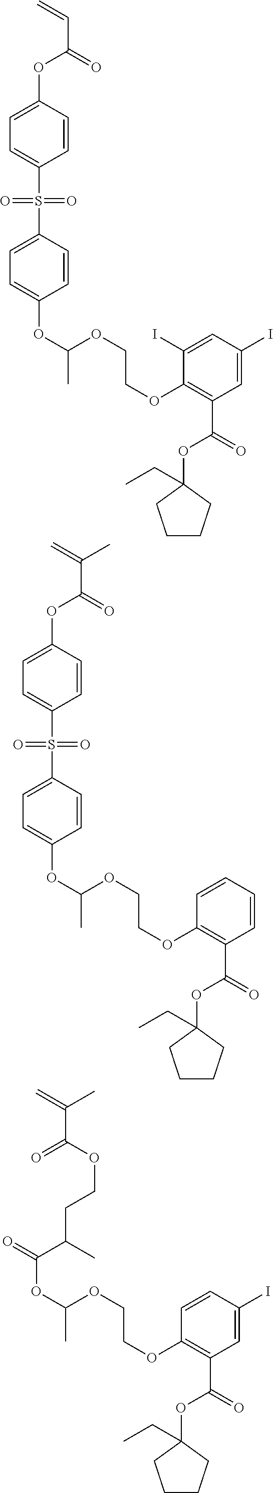 [SMILES: C=CC(=O)Oc1ccc(S(=O)(=O)c2ccc(OC(C)OCCOc3c(I)cc(I)cc3C(=O)OC3(CC)CCCC3)cc2)cc1 ; C=C(C)C(=O)Oc1ccc(S(=O)(=O)c2ccc(OC(C)OCCOc3ccccc3C(=O)OC3(CC)CCCC3)cc2)cc1 ; C=C(C)C(=O)OCCC(C)C(=O)OC(C)OCCOc1ccc(I)cc1C(=O)OC1(CC)CCCC1]

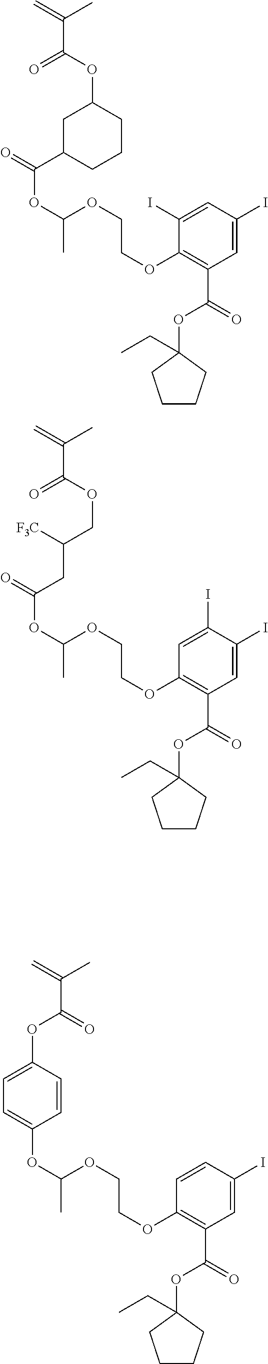 [SMILES: C=C(C)C(=O)OC1CCCC(C(=O)OC(C)OCCOc2c(I)cc(I)cc2C(=O)OC2(CC)CCCC2)C1 ; C=C(C)C(=O)OCC(CC(=O)OC(C)OCCOc1cc(I)c(I)cc1C(=O)OC1(CC)CCCC1)C(F)(F)F ; C=C(C)C(=O)Oc1ccc(OC(C)OCCOc2ccc(I)cc2C(=O)OC2(CC)CCCC2)cc1]

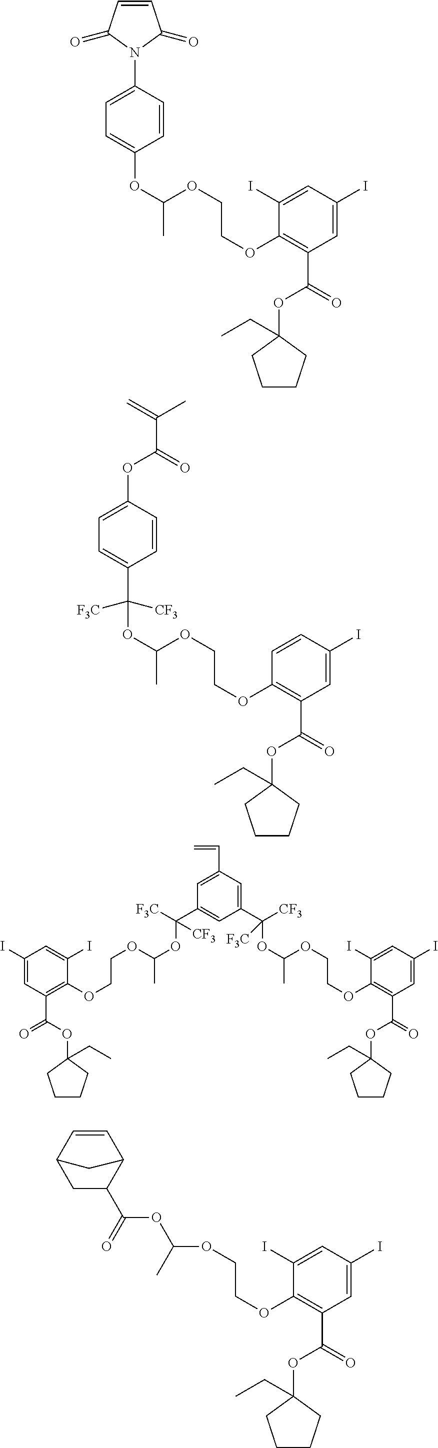

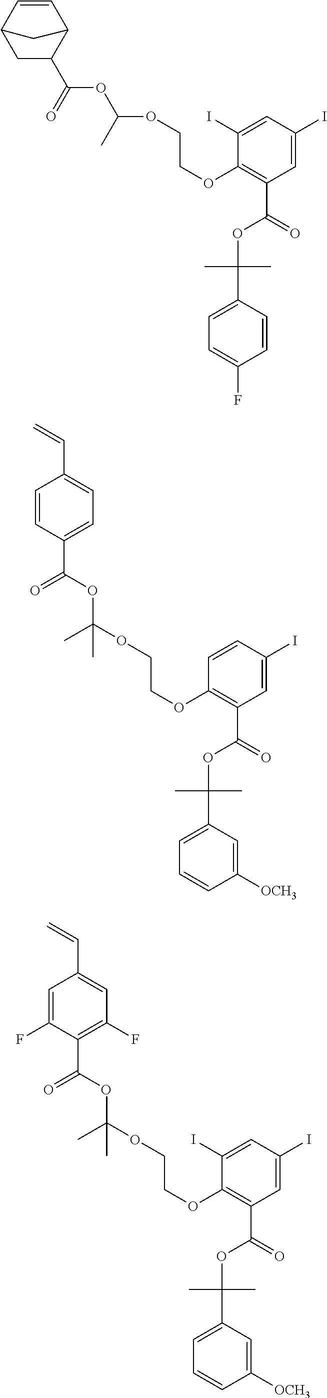 [SMILES: CC(OCCOc1c(I)cc(I)cc1C(=O)OC(C)(C)c1ccc(F)cc1)OC(=O)C1CC2C=CC1C2 ; C=Cc1ccc(C(=O)OC(C)(C)OCCOc2ccc(I)cc2C(=O)OC(C)(C)c2cccc(OC)c2)cc1 ; C=Cc1cc(F)c(C(=O)OC(C)(C)OCCOc2c(I)cc(I)cc2C(=O)OC(C)(C)c2cccc(OC)c2)c(F)c1]

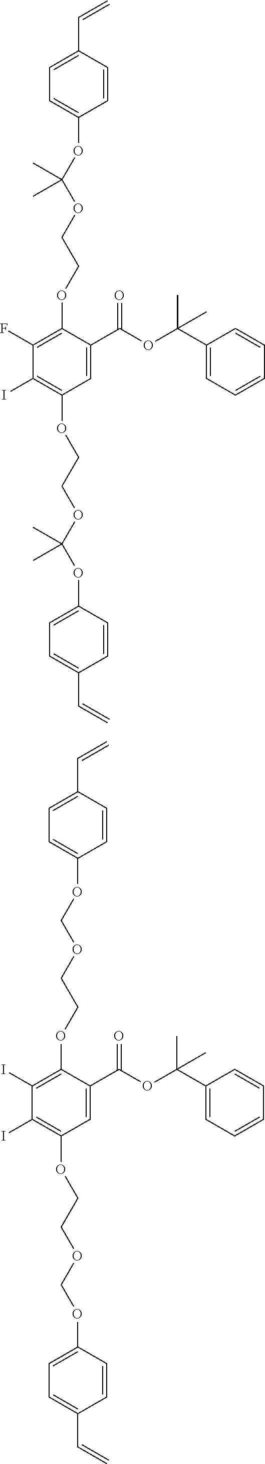

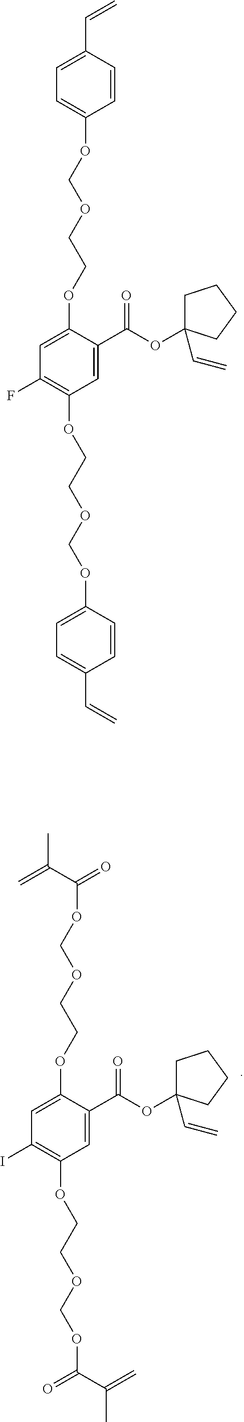 [SMILES: C=Cc1ccc(OCOCCOc2cc(C(=O)OC3(C=C)CCCC3)c(OCCOCOc3ccc(C=C)cc3)cc2F)cc1 ; C=CC1(OC(=O)c2cc(OCCOCOC(=O)C(=C)C)c(I)cc2OCCOCOC(=O)C(=C)C)CCCC1]

The monomers of the invention may be prepared using suitable methods known in the art. In the case of an enol ether reactant, for example, the monomers may be prepared by mixing the monomer precursor and enol ether compound as described herein with a suitable acid catalyst and solvent. Suitable monomer precursors are commercially available or can be made using methods known in the art. The acid catalyst can be an organic or inorganic acid catalyst, for example, methanesulfonic acid, p-toluenesulfonic acid, hydrochloric acid, acetic acid, sulfuric acid, trifluoracetic acid, citric acid, malic acid, formic acid, or combinations thereof. The solvent is typically anhydrous an organic solvent, for example, propylene glycol methyl ether acetate. opropylene glycol methyl ether, &N, N-dimethylformamide, tetrahydrofuran, dimethyl sulfoxide, N-methyl-2-pyrrolidone, 1,4-dioxane, or combinations thereof. The reaction is carried at a temperature and time effective to cause reaction between the reactants to form the acid-sensitive monomer. The reaction temperature is typically from 0 to 150° C., preferably from 25 to 100° C. The reaction time is typically from 1 hour to 96 hours, preferably from 2 to 48 hours. The product monomers can be purified by techniques known in the art such as precipitation, column chromatography recrystallization, or combination thereof.

Monomers prepared from a halomethyl ether reactant may be prepared by mixing the monomer precursor and a halomethyl ether compound as described herein with a suitable acid catalyst and solvent. The monomers of the invention may be prepared using suitable methods known in the art, for example, by mixing the monomer precursor and the halomethyl ether compound with a suitable base and solvent. Suitable monomer precursors are commercially available or can be made using methods known in the art. The base can be an inorganic or organic base, for example, potassium carbonate, cesium carbonate, sodium hydride, N,N-Diisopropylethylamine, or 1,8-Diazabicycloundec-7-ene. The solvent is typically an organic solvent, for example, N,N-dimethylformamide, tetrahydrofuran, dimethyl sulfoxide, N-methyl-2-pyrrolidone, 1,4-dioxane, or combinations thereof. The reaction is conducted at a temperature and time effective to cause reaction between the reactants to form the acid-sensitive monomer. The reaction temperature is typically from 0 to 150° C., preferably from 25 to 100° C. The reaction time is typically from 1 hour to 96 hours, preferably from 2 to 48 hours. The product monomers can be purified by techniques known in the art such as precipitation, column chromatography recrystallization, or combination thereof.

### Acid-Sensitive Polymers

In accordance with a further aspect of the invention, acid-sensitive polymers are provided. As described below, the polymers may be a direct or indirect reaction product of the enol ether compounds or halomethyl ether compounds described herein. The polymers can be a homopolymer or, more typically, a copolymer having a plurality of distinct repeat units, for example, two, three, four, or more distinct repeat units.

The polymer preferably comprises an acid-sensitive repeat unit comprising a moiety of formula (5):

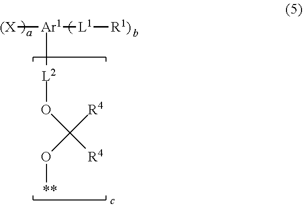

wherein: Ar¹, R¹, R⁴, X, L¹, L², a, b, and c are as described above with reference to formula (3); and ** represents an attachment point to another portion of the repeat unit.

The acid-sensitive repeat unit is preferably of the formula (6):

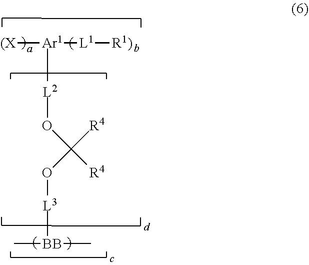

wherein: Ar¹, R¹, R⁴, X, L¹, L², L³, a, b, c, and d are as described above with reference to formula (4); and BB represents a polymer backbone. Typical for BB are polymer backbones formed by polymerization of an ethylenically unsaturated carbon-carbon double bond, preferably a polymerizable vinyl group, for example, a substituted or unsubstituted C₂₋₂₀ alkenyl group, a substituted or unsubstituted norbornyl group, a substituted or unsubstituted (meth)acrylic group, a substituted or unsubstituted vinyl ether group, a substituted or unsubstituted vinyl ketone group, a substituted or unsubstituted vinyl ester group, a substituted or unsubstituted vinyl aromatic group (e.g., styrene), or a substituted or unsubstituted N-maleimide group. Of these, substituted or unsubstituted vinyl aromatic groups and substituted or unsubstituted (meth)acrylate groups are typical.

The acid-sensitive repeat unit is typically of the following formula (6a) or (6b):

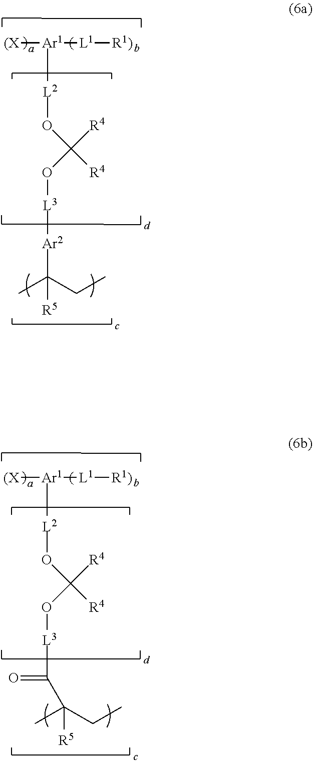

wherein: Ar¹, Ar², R¹, R⁴, R⁵, X, L¹, L², L³, a, b, c, and d are as described above with reference to formulae (4a) and (4b).

The acid-sensitive repeat unit is typically present in the polymer in an amount from 5 to 100 mol %, 5 to 90 mol %, more typically from 5 to 50 mol %, still more typically from 10 to 30 mol %, based on total repeating units in the polymer.

When the acid-sensitive polymer is in the form of a copolymer, it may include one or more additional structurally different acid-sensitive repeat units as described above, or may include one or more other types of repeat units. Other suitable additional repeat units include, for example, those comprising an acid-labile group, a base-decomposable group, a hydroxy aryl group, a fluoroalcohol group, or a lactone group.

In some aspects, the polymer may include an additional repeating unit having an aromatic group, wherein the aromatic group may be substituted or unsubstituted. The aromatic group is a monocyclic or polycyclic C₅₋₆₀ aromatic group optionally comprising one or more aromatic ring heteroatoms chosen from N, O, S, or a combination thereof. When the aromatic group is polycyclic, the ring or ring groups may be fused (such as naphthyl or the like), directly linked (such as biaryls such as biphenyl), bridged by a heteroatom (such as triphenylamino or diphenylene ether), and/or may include a combination of fused and directly linked rings (such as binaphthyl or the like).

The monocyclic or polycyclic C₅₋₆₀ aromatic group can be substituted or unsubstituted. Exemplary substituents include, but are not limited to, substituted or unsubstituted C₁₋₃₀ alkyl, substituted or unsubstituted C₁₋₃₀ haloalkyl, substituted or unsubstituted C₃₋₃₀ cycloalkyl, substituted or unsubstituted C₁₋₃₀ heterocycloalkyl, substituted or unsubstituted C₂₋₃₀ alkenyl, substituted or unsubstituted C₂₋₃₀ alkynyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ heteroarylalkyl, halogen, —OR⁵¹, —SR⁵², or —NR⁵³R⁵⁴, wherein R⁵¹ to R⁵⁴ are each independently hydrogen, or substituted or unsubstituted C₁₋₃₀ alkyl, substituted or unsubstituted C₃₋₃₀ cycloalkyl, substituted or unsubstituted C₂₋₃₀ heterocycloalkyl, substituted or unsubstituted C₆₋₃₀ aryl, substituted or unsubstituted C₇₋₃₀ arylalkyl, substituted or unsubstituted C₃₋₃₀ heteroaryl, or substituted or unsubstituted C₄₋₃₀ heteroarylalkyl. Preferably, the aromatic group is a substituted C₆₋₃₀ aryl group or a substituted C₇₋₃₀ heteroaryl group, wherein the aromatic group is substituted with a heteroatom-containing substituent group, such as —OR⁵¹, —SR⁵², or —NR⁵³R⁵⁴, wherein R⁵¹ to R⁵⁴ are each independently hydrogen, or substituted or unsubstituted C₁₋₁₀ alkyl, substituted or unsubstituted C₃₋₂₀ cycloalkyl, substituted or unsubstituted C₂₋₂₀ heterocycloalkyl, substituted or unsubstituted C₆₋₃₀ aryl, substituted or unsubstituted C₇₋₃₀ arylalkyl, substituted or unsubstituted C₃₋₃₀ heteroaryl, or substituted or unsubstituted C₄₋₃₀ heteroarylalkyl.

The repeating unit having the aromatic group is typically present in the polymer in an amount from 5 to 80 mol %, more typically from 10 to 50 mol %, still more typically from 10 to 40 mol %, based on total repeating units in the polymer.

The one or more additional repeat units are not particularly limited, and may be chosen, for example, from the optional repeat units described below with respect to the further method for forming polymers of the invention described below, or other repeat units known in the art for use in photoresist polymers.

The acid-sensitive polymer may comprise one or more additional repeat units comprising an acid-labile group, for example, an acid-labile ester group or an acetal group. Suitable monomers for these repeat units include, for example, the following:

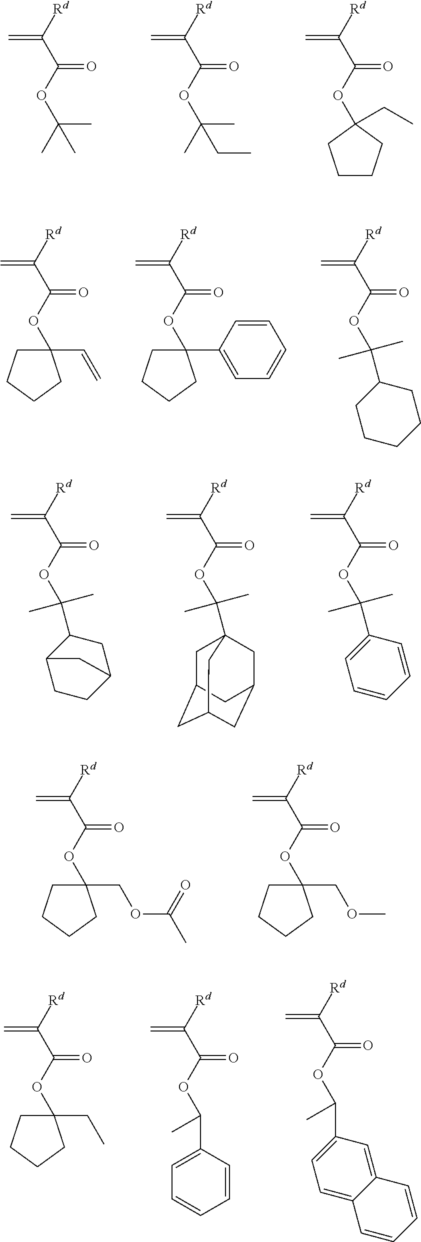

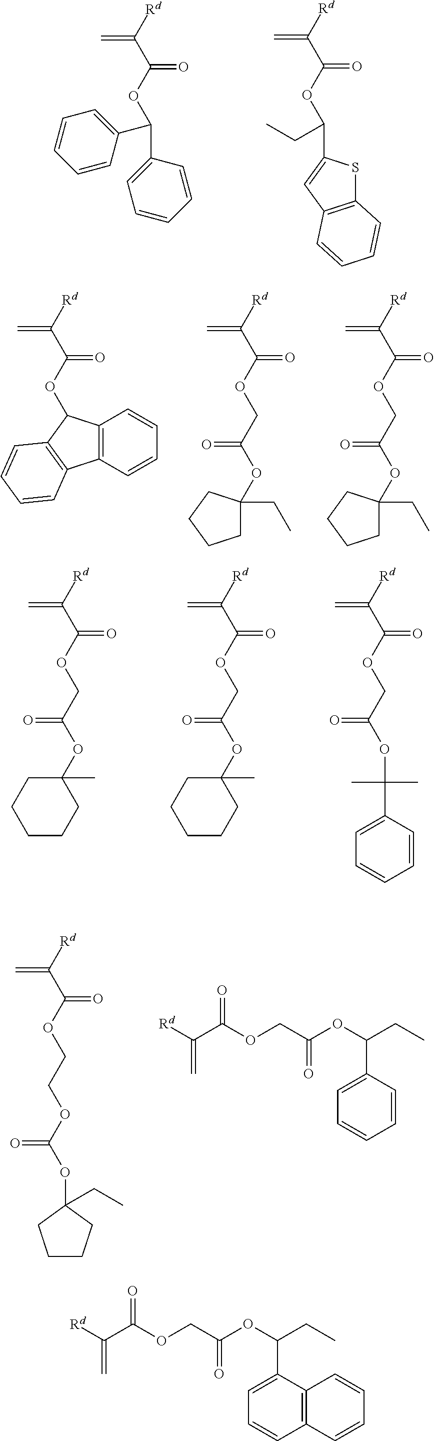

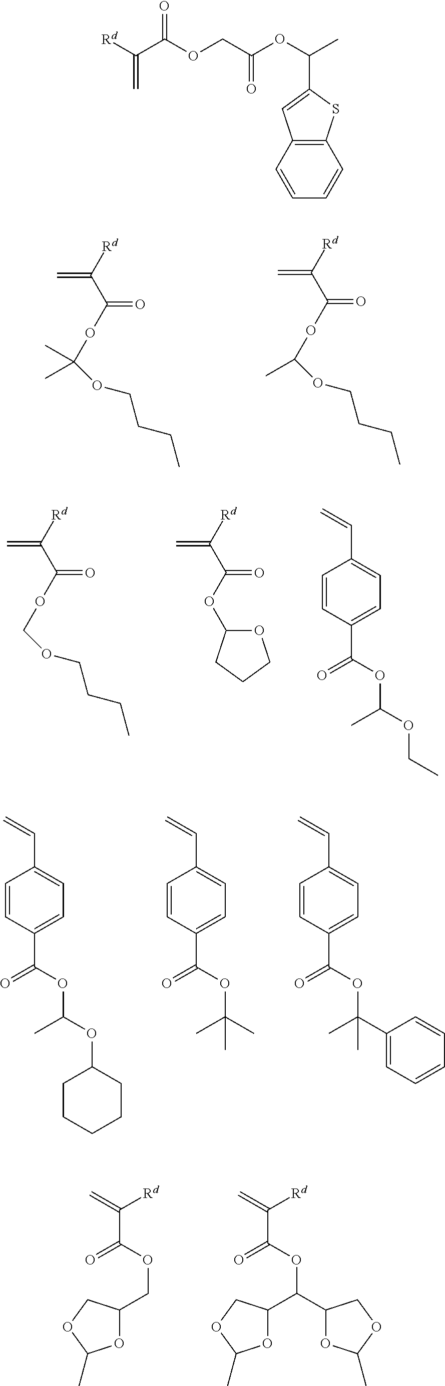

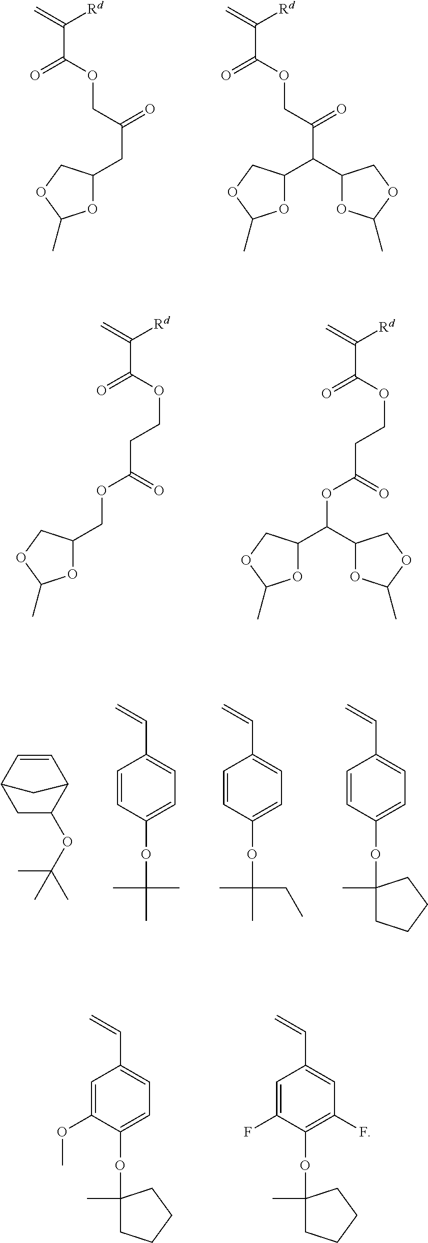

wherein R^{d} is hydrogen, fluorine, C₁₋₅ alkyl, or C₁₋₅ fluoroalkyl, typically hydrogen or methyl.

Repeating units having an acid-labile group are typically present in the acid-sensitive polymer in an amount from 10 to 80 mol %, more typically from 25 to 75 mol %, still more typically from 30 to 70 mol %, based on total repeating units in the acid-sensitive polymer.

In some aspects, the acid-sensitive polymer may include one or more additional repeat units having an aromatic group, wherein the aromatic group may be substituted or unsubstituted. The aromatic group is a monocyclic or polycyclic C₅₋₆₀ aromatic group optionally comprising one or more aromatic ring heteroatoms chosen from N, O, S, or a combination thereof. When the aromatic group is polycyclic, the ring or ring groups may be fused (such as naphthyl or the like), directly linked (such as biaryls such as biphenyl), bridged by a heteroatom (such as triphenylamino or diphenylene ether), and/or may include a combination of fused and directly linked rings (such as binaphthyl or the like).

The monocyclic or polycyclic C₅₋₆₀ aromatic group can be substituted or unsubstituted. Exemplary substituents include, but are not limited to, substituted or unsubstituted C₁₋₃₀ alkyl, substituted or unsubstituted C₁₋₃₀ haloalkyl, substituted or unsubstituted C₃₋₃₀ cycloalkyl, substituted or unsubstituted C₁₋₃₀ heterocycloalkyl, substituted or unsubstituted C₂₋₃₀ alkenyl, substituted or unsubstituted C₂₋₃₀ alkynyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ heteroarylalkyl, halogen, —OR⁵¹, —SR⁵², or —NR⁵³R⁵⁴, wherein R⁵¹ to R⁵⁴ are each independently hydrogen, or substituted or unsubstituted C₁₋₃₀ alkyl, substituted or unsubstituted C₃₋₃₀ cycloalkyl, substituted or unsubstituted C₂₋₃₀ heterocycloalkyl, substituted or unsubstituted C₆₋₃₀ aryl, substituted or unsubstituted C₇₋₃₀ arylalkyl, substituted or unsubstituted C₃₋₃₀ heteroaryl, or substituted or unsubstituted C₄₋₃₀ heteroarylalkyl. Preferably, the aromatic group is a substituted C₆₋₃₀ aryl group or a substituted C₇₋₃₀ heteroaryl group, wherein the aromatic group is substituted with a heteroatom-containing substituent group, such as —OR⁵¹, —SR⁵², or —NR⁵³R⁵⁴, wherein R⁵¹ to R⁵⁴ are each independently hydrogen, or substituted or unsubstituted C₁₋₁₀ alkyl, substituted or unsubstituted C₃₋₂₀ cycloalkyl, substituted or unsubstituted C₂₋₂₀ heterocycloalkyl, substituted or unsubstituted C₆₋₃₀ aryl, substituted or unsubstituted C₇₋₃₀ arylalkyl, substituted or unsubstituted C₃₋₃₀ heteroaryl, or substituted or unsubstituted C₄₋₃₀ heteroarylalkyl.

If used, the additional repeat unit having an aromatic group is typically present in the acid-sensitive polymer in an amount from 5 to 80 mol %, more typically from 10 to 50 mol %, still more typically from 10 to 40 mol %, based on total repeating units in the acid-sensitive polymer.

The acid-sensitive polymer may include a repeating unit comprising a lactone group. Suitable repeating units may, for example, be derived from a monomer of formula (6a) or formula (6b):

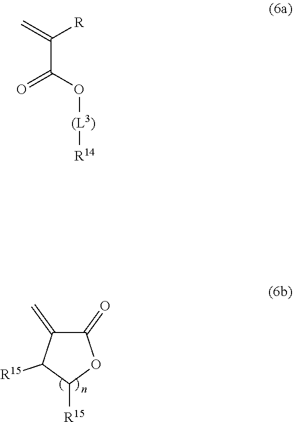

In formula (6a), R is hydrogen, fluorine, cyano, a substituted or unsubstituted C₁₋₁₀ alkyl, or a substituted or unsubstituted C₁₋₁₀ fluoroalkyl. Preferably, R is hydrogen, fluorine, or substituted or unsubstituted C₁₋₅ alkyl, typically methyl. L³ may be a single bond or a divalent linking group comprising one or more of substituted or unsubstituted C₁₋₃₀ alkylene, substituted or unsubstituted C₁₋₃₀ heteroalkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted C₇₋₃₀ arylalkylene, or substituted or unsubstituted C₁₋₃₀ heteroarylene, or substituted or unsubstituted C₃₋₃₀ heteroarylalkylene, wherein L³ optionally may further include one or more groups chosen, for example, from —O—, —C(O)—, —C(O)—O—, —S—, —S(O)₂—, and —N(R⁴⁴)—S(O)₂—, wherein R⁴⁴ may be hydrogen, a straight chain or branched C₁₋₂₀ alkyl, monocyclic or polycyclic C₃₋₂₀ cycloalkyl, or monocyclic or polycyclic C₃₋₂₀ heterocycloalkyl. R¹⁴ may be a monocyclic, polycyclic, or fused polycyclic C₄₋₂₀ lactone-containing group. In formula (6b), R¹⁵ is hydrogen or a non-hydrogen substituent, typically substituted or unsubstituted C₁₋₁₀ alkyl and n is 1 or 2.

Suitable exemplary lactone-containing monomers of formulas (6a) and (6b) include the following:

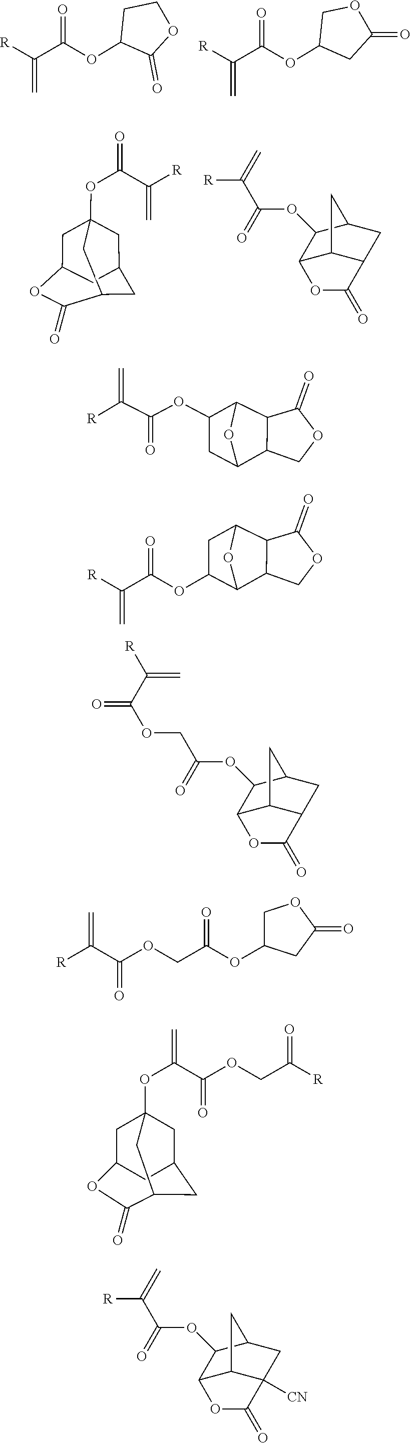

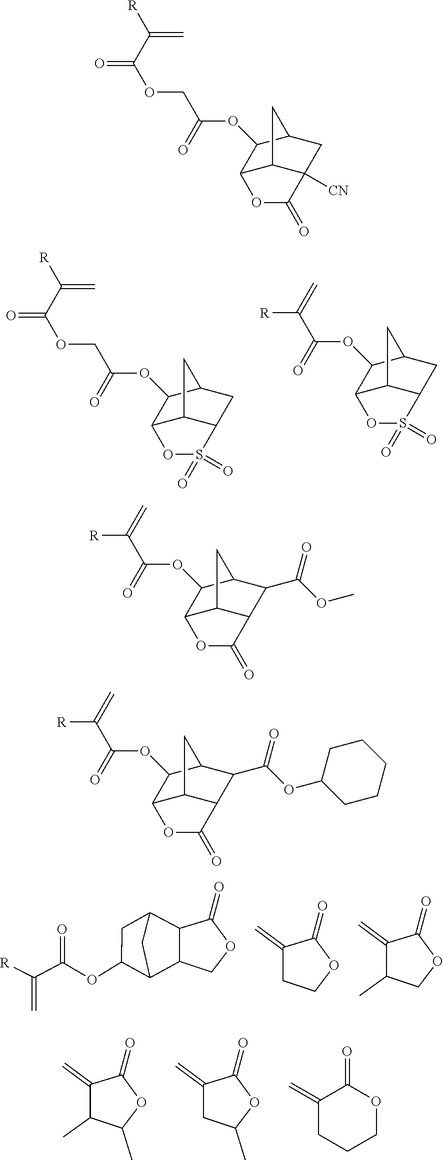

wherein R is as described above. When present, the acid-sensitive polymer typically comprises a lactone repeating unit in an amount from 5 to 60 mol %, typically 20 to 55 mol %, more typically 25 to 50 mol % based on total repeating units in the acid-sensitive polymer.

The acid-sensitive polymer may include a base-soluble repeating unit having a pKa of less than or equal to 12. For example, the base-soluble repeating unit can be derived from a monomer of formula (7):

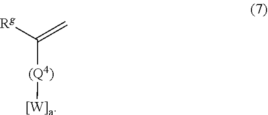

In formula (7), Re may be hydrogen, fluorine, cyano, a substituted or unsubstituted C₁₋₁₀ alkyl, or a substituted or unsubstituted C₁₋₁₀ fluoroalkyl. Preferably, R⁸ is hydrogen, fluorine, or substituted or unsubstituted C₁₋₅ alkyl, typically methyl. Q⁴ may comprise one or more of substituted or unsubstituted C₁₋₃₀ alkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted divalent C₇₋₃₀ arylalkyl, substituted or unsubstituted C₁₋₃₀ heteroarylene, or substituted or unsubstituted divalent C₃₋₃₀ heteroarylalkyl, or —C(O)—O—. W is a base-soluble group and can be chosen, for example, from hydroxyl (—OH); —C(O)—OH; a fluorinated alcohol such as —C (CF₃)₂OH; an imide; or —NH—S(O)₂—Y¹ where Y¹ is C₁₋₄ alkyl or fluoroalkyl, typically C₁₋₄ perfluoroalkyl. In formula (7), a is 1, 2, or 3.

Non-limiting examples of monomers of formula (7) include:

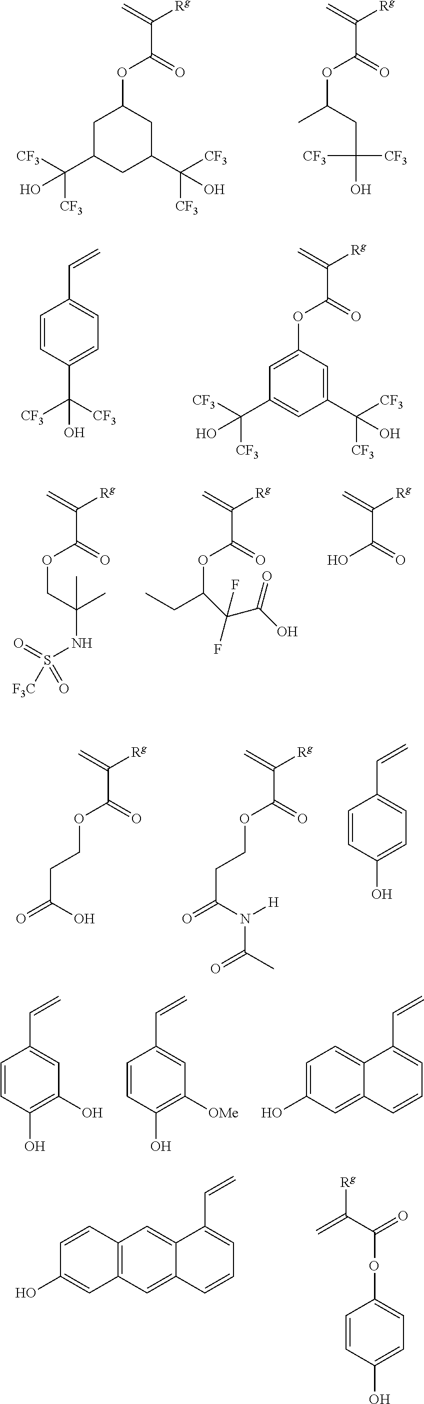

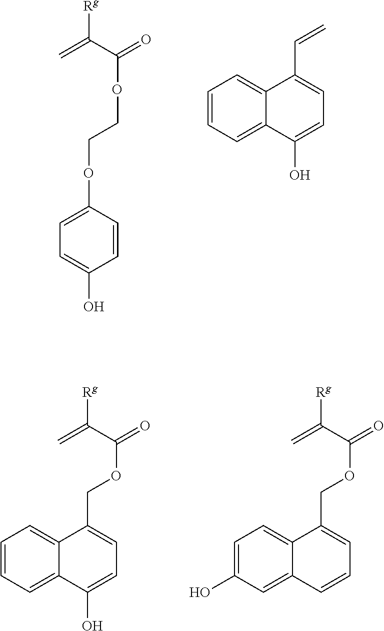

wherein R⁸ is as described above. When present, the acid-sensitive polymer typically comprises the above-described base-soluble repeating unit in an amount from 5 to 60 mol %, typically 20 to 55 mol %, more typically 25 to 50 mol % based on total repeating units in the acid-sensitive polymer.

The acid-sensitive polymer may optionally include one or more additional repeating units. The additional repeating units may include, for example, one or more additional units for purposes of adjusting properties of the photoresist composition, such as etch rate and solubility. The additional units may include, for example, one or more of styrene, (meth)acrylate, vinyl ether, vinyl ketone, or vinyl ester type units. The one or more additional repeating units if present in the acid-sensitive polymer may be used in an amount of up to 90 mol %, typically from 3 to 50 mol %, based on total repeating units of the acid-sensitive polymer.

Non-limiting examples of the acid-sensitive polymer include the following:

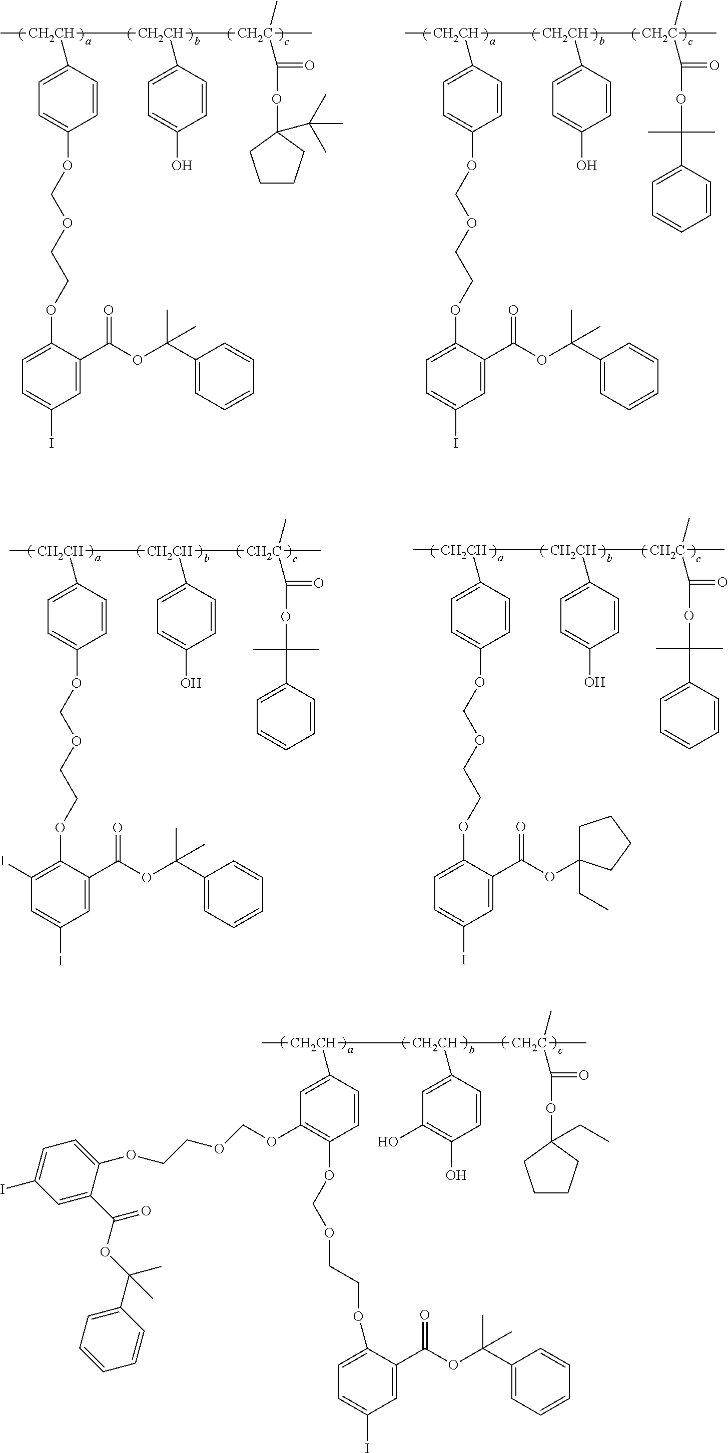

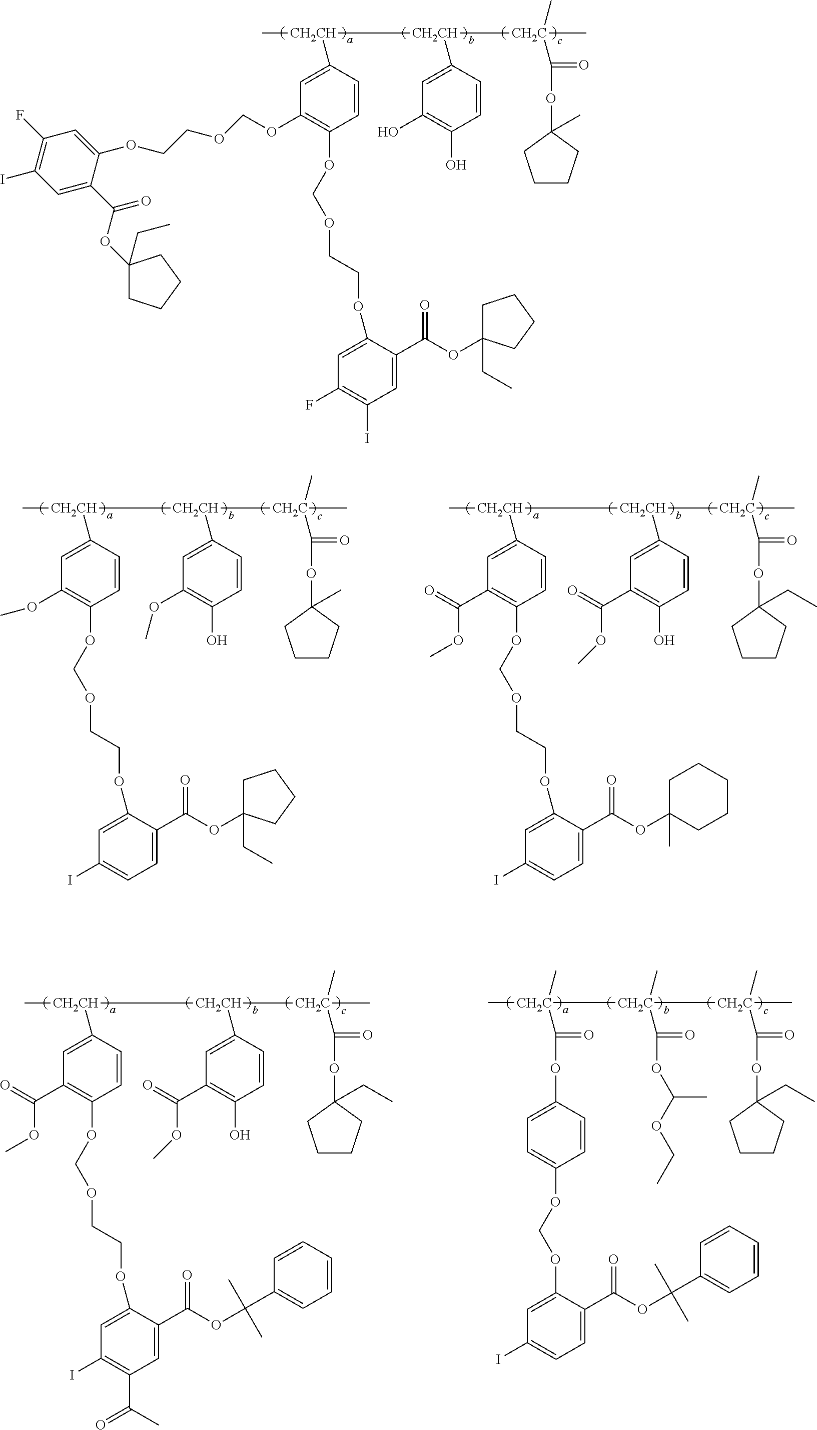

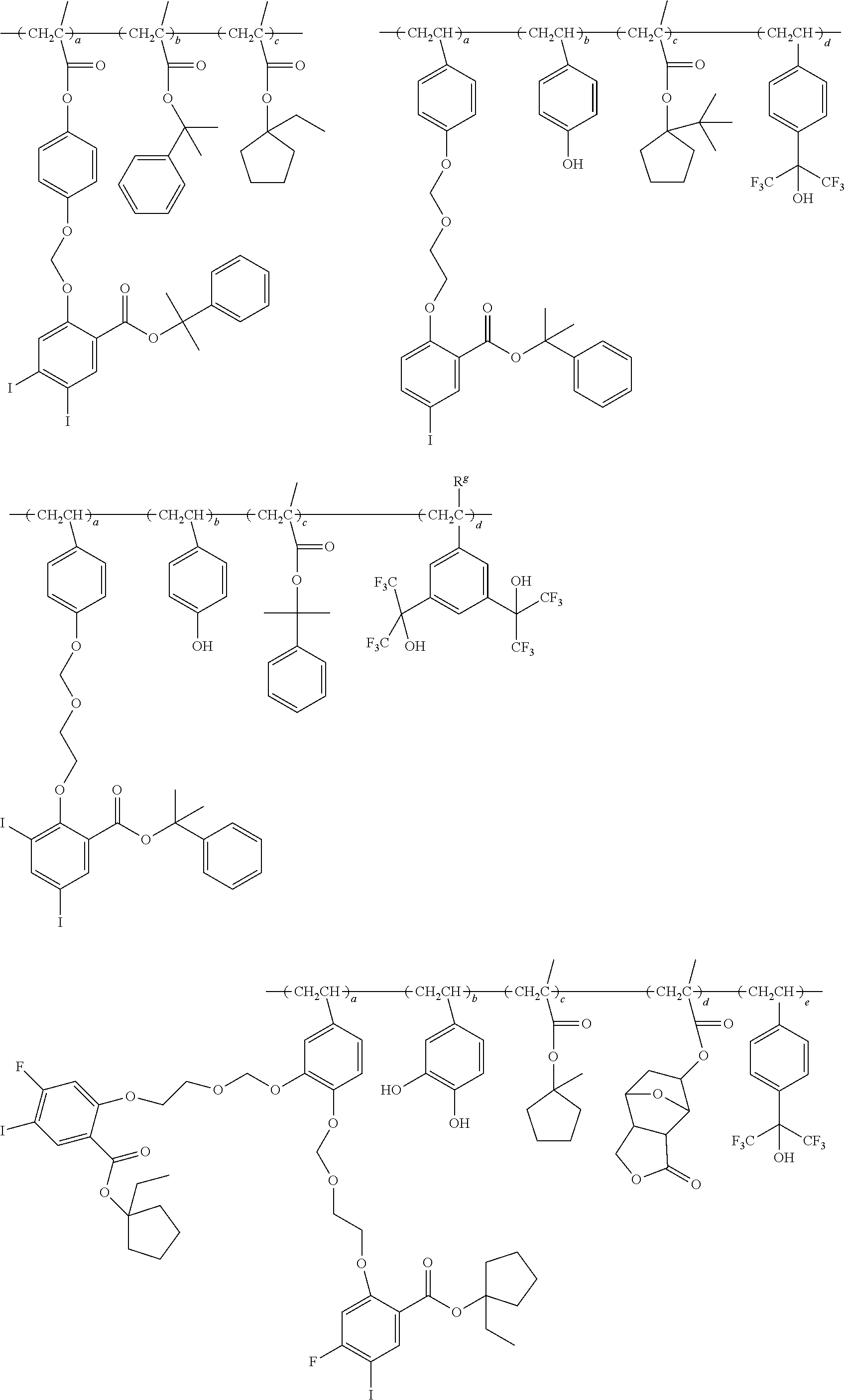

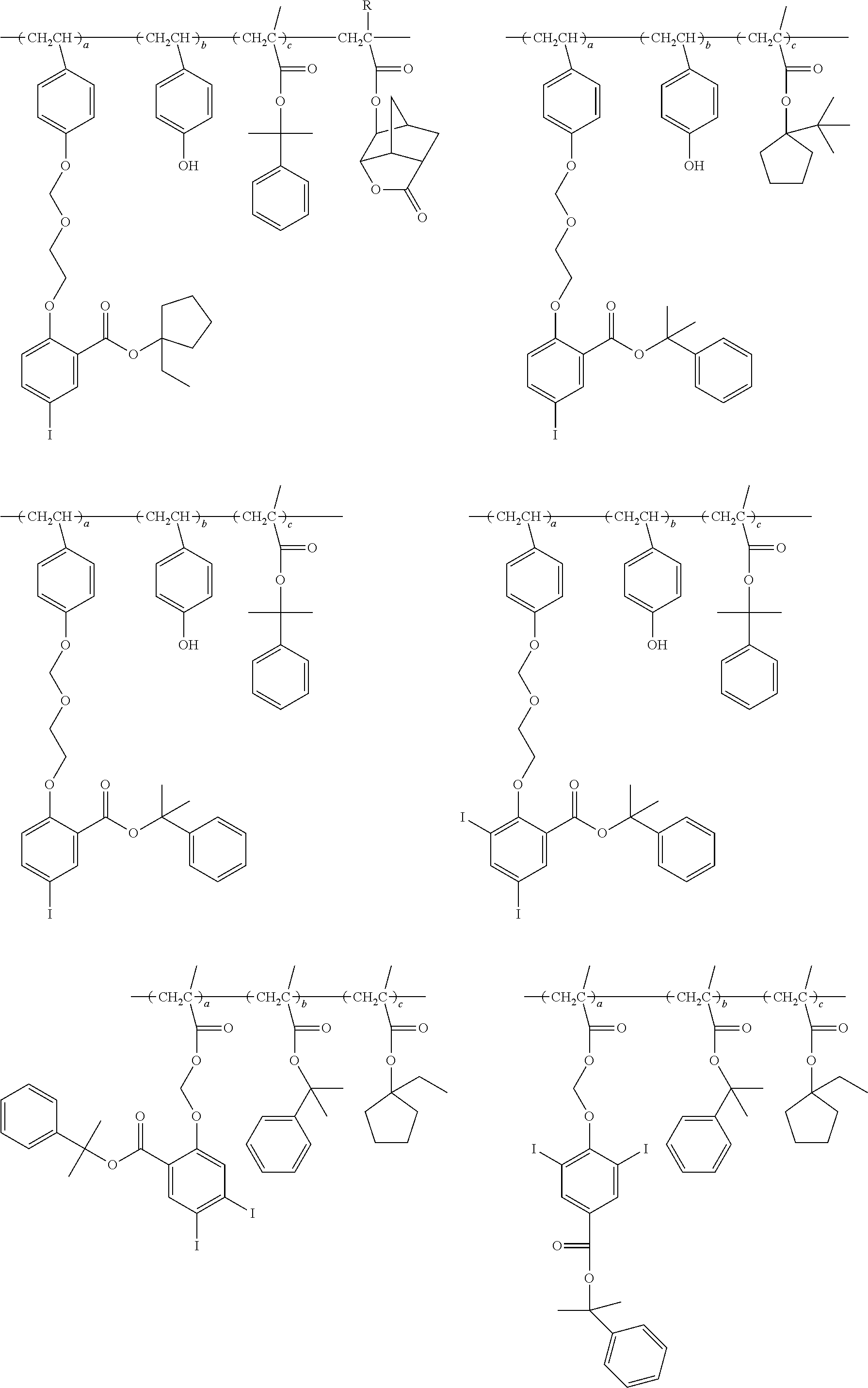

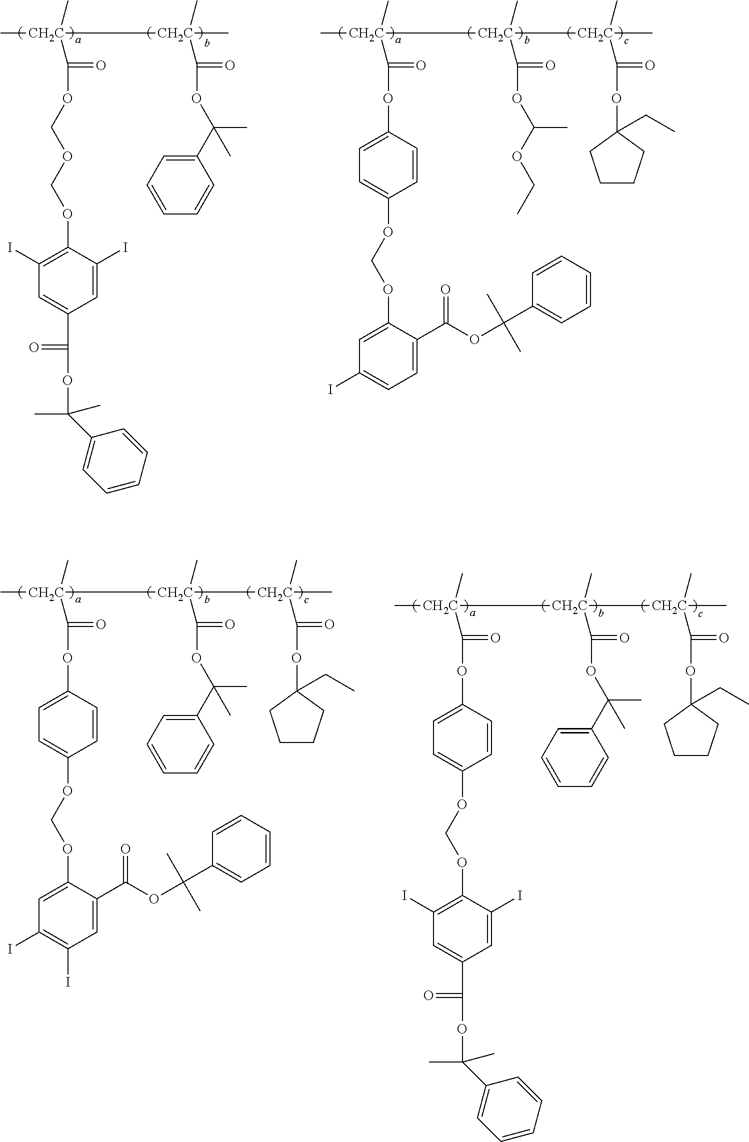

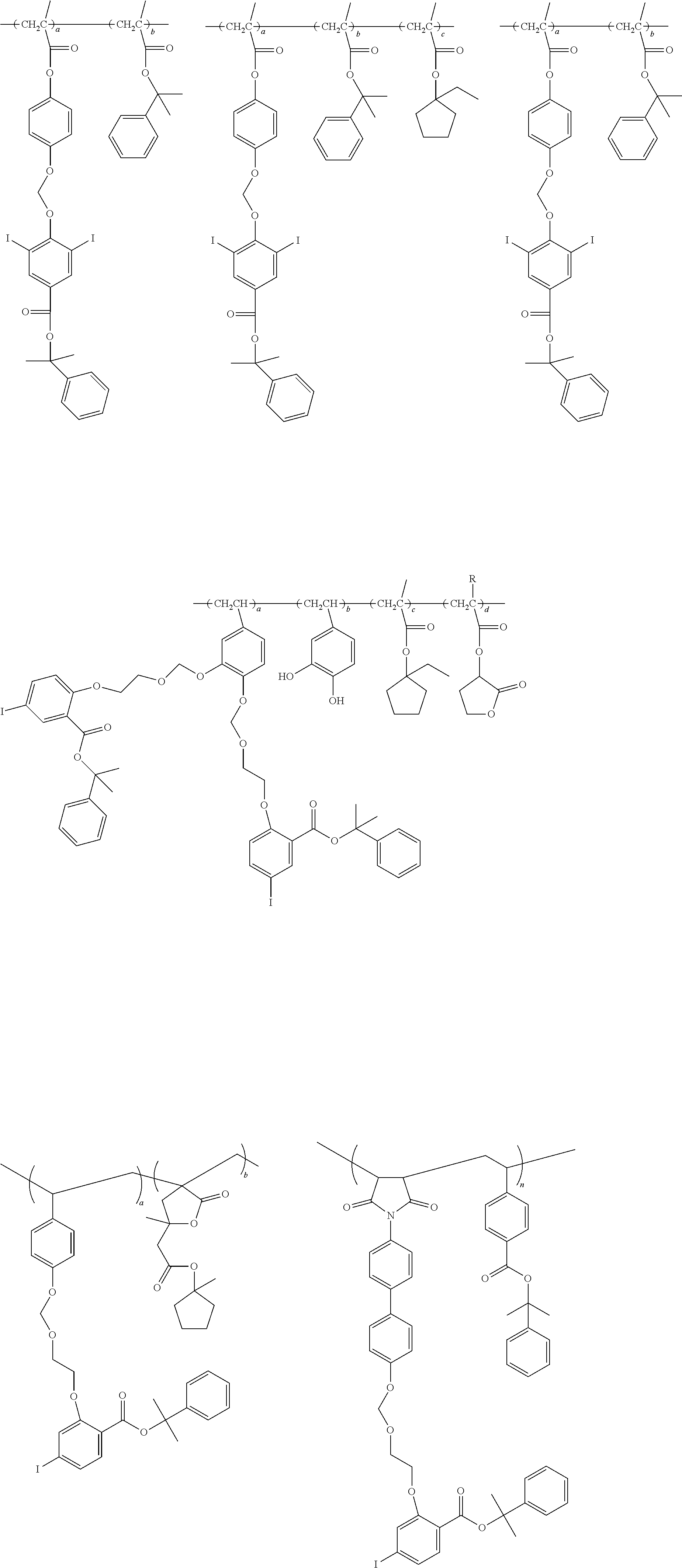

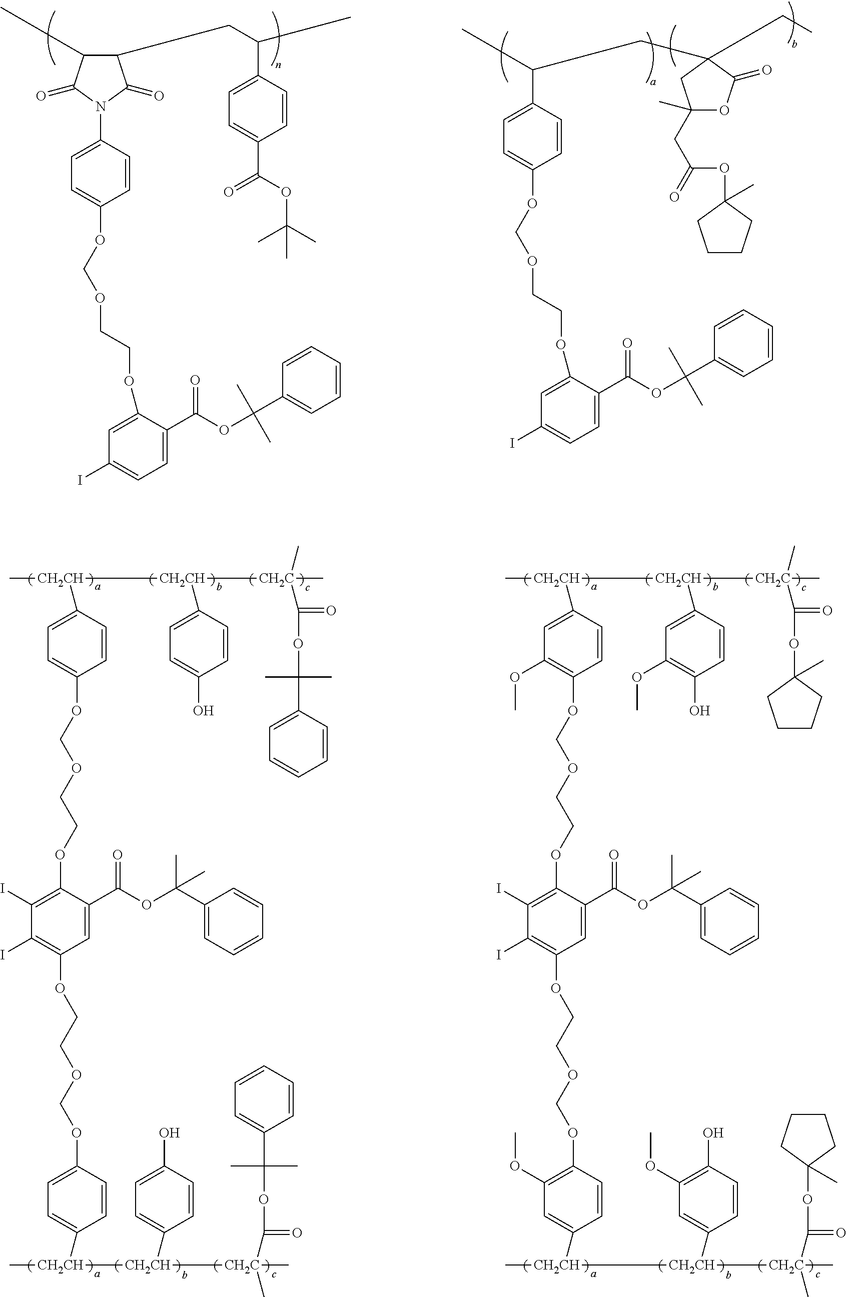

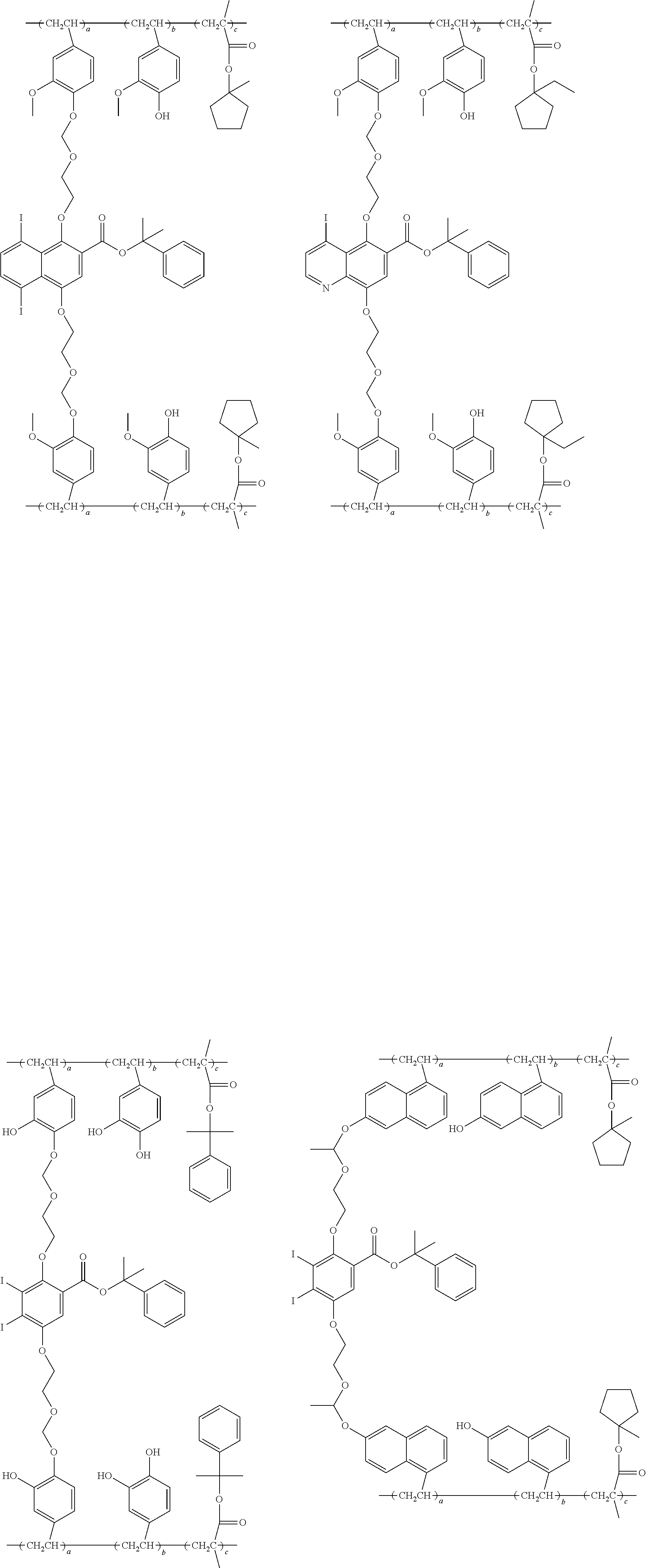

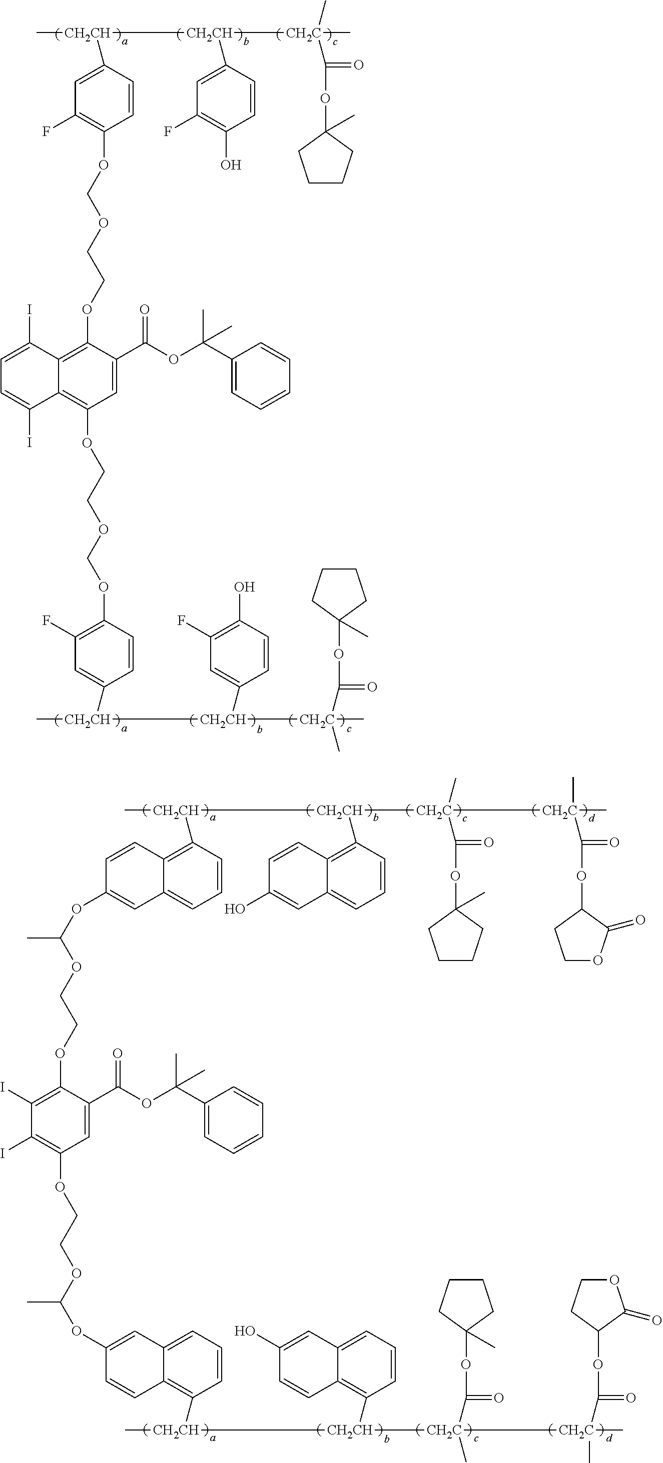

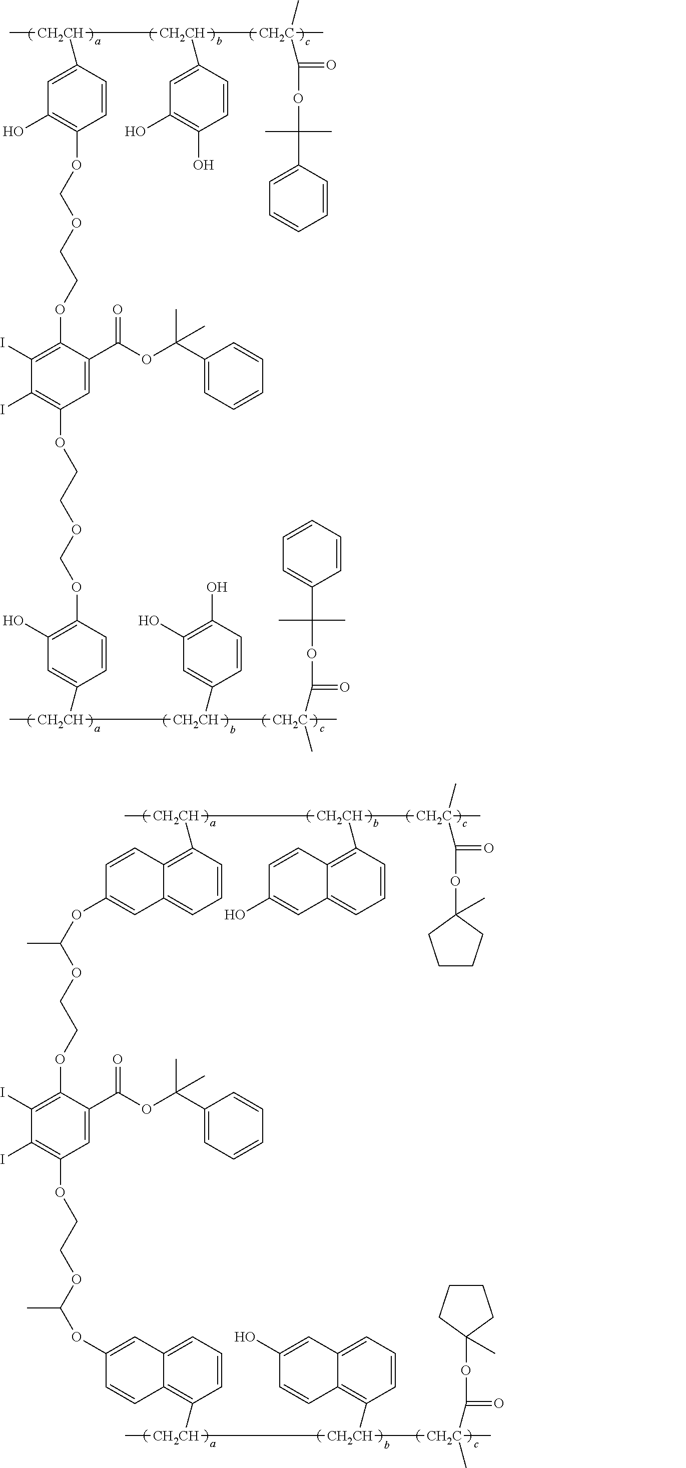

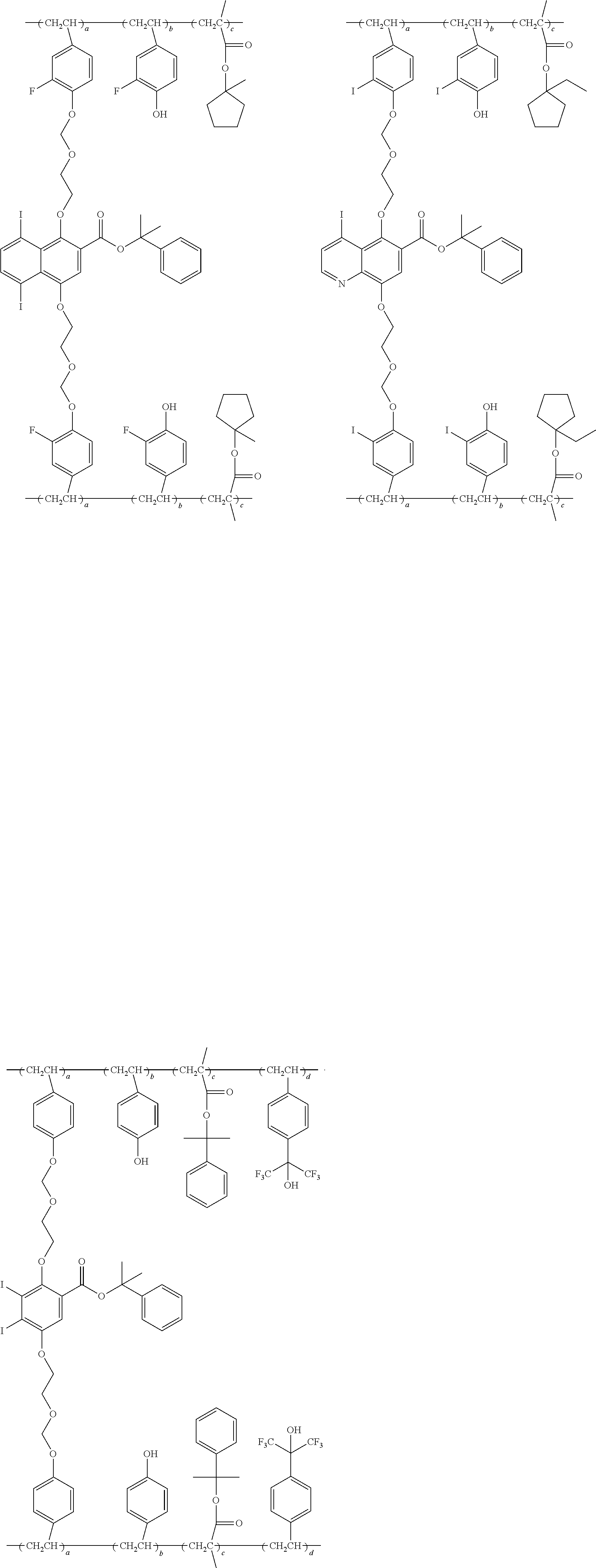

wherein a, b, c, and d represent molar fractions of the corresponding repeating units, with the sum of the molar fractions of all repeat units within each polymer being 1.

The acid-sensitive polymer typically has a weight average molecular weight (Mw) of 1,000 to 50,000 Daltons (Da), specifically 2,000 to 30,000 Da, more specifically 3,000 to 20,000 Da, still more specifically 3,000 to 10,000 Da. The polydispersity index (PDI) of the acid-sensitive polymer, which is the ratio of Mw to number average molecular weight (Mn) is typically 1.1 to 3, specifically 1.1 to 2. Molecular weight values are determined by gel permeation chromatography (GPC) using polystyrene standards.

Polymers of the invention can be prepared by various methods. For example, the polymers can be made by polymerization of an acid-sensitive monomer of the invention as described above, together with one or more optional comonomers as described above. The polymers may be prepared using any suitable methods in the art, for example, free-radical polymerization, anionic polymerization, cationic polymerization, and the like. One or more monomers corresponding to the repeating units of the precursor polymer may, for example, be combined or fed separately, using a suitable solvent and initiator, and polymerized in a reactor. Polymerization may be performed under suitable conditions, such as by heating at an effective temperature, irradiation with actinic radiation at an effective wavelength, or a combination thereof. Where the polymer includes a hydroxy-substituted vinyl aromatic repeat unit, such repeat unit may be formed by polymerization of a hydroxy-substituted vinyl aromatic monomer, for example, hydroxy styrene. The hydroxy-substituted vinyl aromatic repeat unit may alternatively be prepared by use of a protected hydroxy aromatic repeat unit, for example, repeat units of acetoxy styrene. The acetoxy groups are then removed by reaction with a base to produce the hydroxy aromatic repeat unit. The base is then removed, for example, by treatment of the crude polymer with an acidic ion exchange resin.

The acid-sensitive polymers may further be made by reacting (i) an enol ether compound or a halomethyl compound as described herein, with (ii) a precursor polymer comprising a repeat unit comprising a group chosen from one or more of —OH or —C(O)OH. The reaction product comprises an acetal or ketal linker formed by reaction between an enol ether moiety of the compound and the —OH or —C(O)OH group of the repeat unit, thereby forming a modified repeat unit.

The precursor polymer comprises one or more repeat units with an —OH or —C(O)OH group, with which the enol ether compound can react. Suitable such repeat units for the precursor polymer include the following:

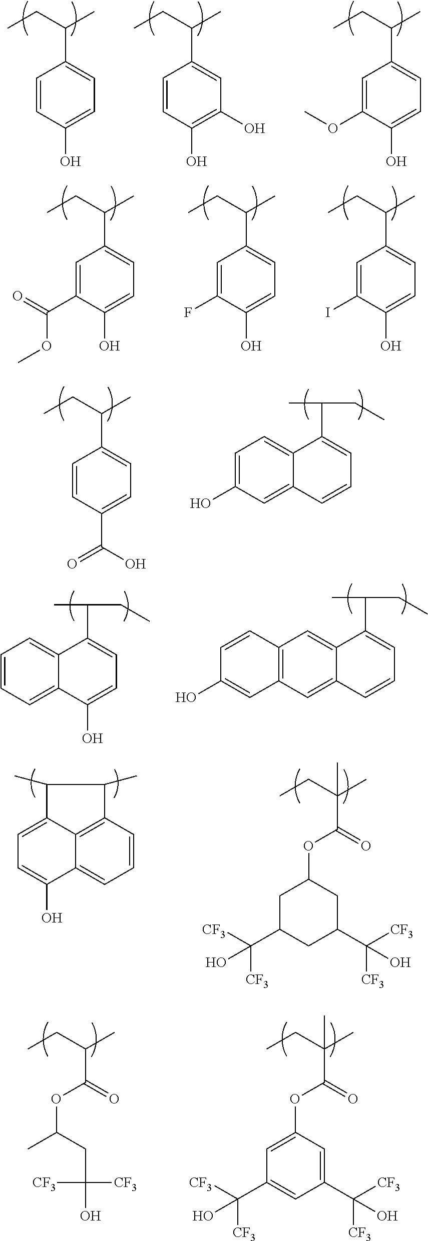

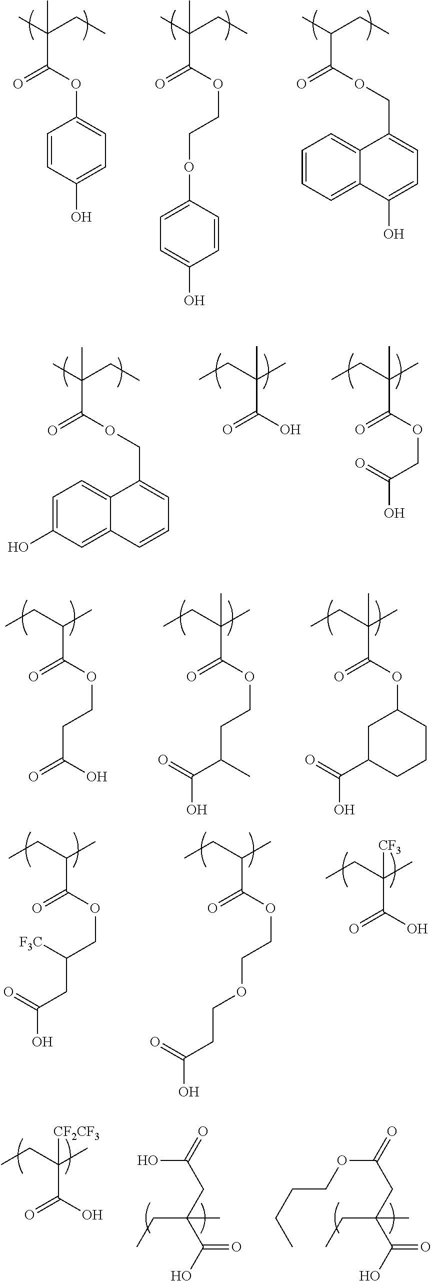

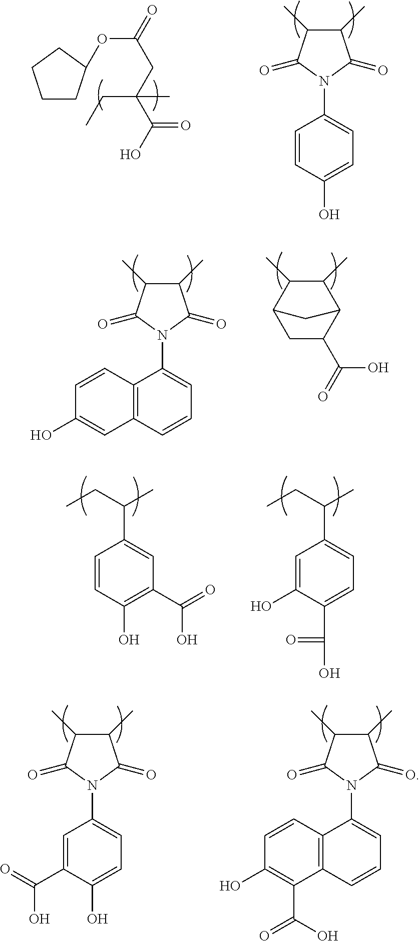

The repeating units of the precursor polymer comprising an —OH group or a —C(O)OH group are typically present in the acid-sensitive polymer in an amount from 1 to 100 mol %, 5 to 100 mol %, 5 to 90 mol %, more typically from 5 to 50 mol %, still more typically from 10 to 30 mol %, based on total repeating units of the precursor polymer.

The precursor polymer may be prepared using any suitable method in the art, for example, free-radical polymerization, anionic polymerization, cationic polymerization, and the like. One or more monomers corresponding to the repeating units of the precursor polymer may, for example, be combined, or fed separately, using suitable solvent and initiator, and polymerized in a reactor. Polymerization can be conducted under any suitable conditions, such as by heating at an effective temperature, irradiation with actinic radiation at an effective wavelength, or a combination thereof. Where the precursor polymer includes a hydroxy-substituted vinyl aromatic repeat unit, such repeat unit may be formed by polymerization of a hydroxy-substituted vinyl aromatic monomer, for example, hydroxy styrene, or by use of a protected hydroxy aromatic repeat unit, for example, repeat units of acetoxy styrene, as described above with reference to the first polymer synthesis method.

The precursor polymer may be reacted with an enol ether compound of a halomethyl ether compound of the invention using suitable methods known in the art. For the enol ether compound, the polymers of the invention may be prepared by mixing the precursor polymer and enol ether compound with a suitable acid catalyst and solvent. The acid catalyst can be an organic or inorganic acid catalyst, for example, methanesulfonic acid, p-toluenesulfonic acid, hydrochloric acid, acetic acid, sulfuric acid, trifluoracetic acid, citric acid, malic acid, formic acid, or combinations thereof. The solvent is typically an organic solvent, for example, propylene glycol methyl ether acetate, [symbol] ropylene glycol methyl ether, % N,N-dimethylformamide, tetrahydrofuran, dimethyl sulfoxide, N-methyl-2-pyrrolidone, 1,4-dioxane, or combinations thereof. The reaction is carried at a temperature and time effective to cause reaction of the reactants in the reaction mixture to form the polymer of the invention. The reaction temperature is typically from 0 to 150° C., preferably from 25 to 100° C. The reaction time is typically from 1 hour to 96 hours, preferably from 2 to 48 hours. The product polymers can be purified by techniques known in the art such as column chromatography.

For the halomethyl ether compound, the polymers of the invention may be prepared, for example, by mixing the polymer precursor and a halomethyl ether compound of the invention with a suitable base and solvent. The base can be an organic or inorganic base, for example, potassium carbonate, cesium carbonate, sodium hydride, N,N-Diisopropylethylamine (DIPEA), or 1,8-Diazabicycloundec-7-ene. The solvent is typically an organic solvent, for example, N,N-dimethylformamide, tetrahydrofuran, dimethyl sulfoxide, N-methyl-2-pyrrolidone, 1,4-dioxane, or combinations thereof. The reaction is carried at a temperature and time effective to cause reaction of the reactants in the reaction mixture to form the acid-sensitive monomer. The reaction temperature is typically from 0 to 150° C., preferably from 25 to 100° C. The reaction time is typically from 1 hour to 96 hours, preferably from 2 to 48 hours. The product polymer can be purified by techniques known in the art such as precipitation.

### Photoresist Compositions

The photoresist compositions of the invention comprise a polymer as described above and a solvent, and may include one or more additional optional components. The polymer may serve various functions in the composition depending on the nature of the polymer and other components of the photoresist composition. For example, in a preferred aspect, the polymer may serve as the photoresist matrix material. In another aspect, the polymer may function as a surface active polymer, segregating from other components of the photoresist composition during the coating process due to relative surface energy properties of the polymer and other components.

The photoresist composition may further include a photoacid generator (PAG). The PAG may be in ionic or non-ionic form. The PAG may be in polymeric or non-polymeric form. In polymeric form, the PAG may be present as a moiety in a repeating unit of the above-described polymer or a different polymer, the repeating unit being derived from a polymerizable PAG monomer.

Suitable PAG compounds may be of the formula G⁺A⁻, wherein G⁺ is a photoactive cation and A⁻ is an anion that can generate a photoacid. The photoactive cation is preferably chosen from onium cations, preferably iodonium or sulfonium cations. Particularly suitable anions include those whose conjugated acids have a pKa of from −15 to 10. The anion is typically an organic anion having a sulfonate group or a non-sulfonate-type group, such as sulfonamidate, sulfonimidate, methide, or borate.

In some embodiments, the photoactive cation may be a sulfonium cation or an iodonium cation. For example, the photoactive cation may be a sulfonium cation of formula (8) or an iodonium cation of formula (9):

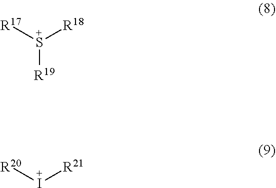

In formulae (8) and (9), R¹⁷ to R²¹ may each independently be substituted or unsubstituted C₁₋₂₀ alkyl, substituted or unsubstituted C₃₋₂₀ cycloalkyl, substituted or unsubstituted C₂₋₂₀ alkenyl, substituted or unsubstituted C₆₋₃₀ aryl, substituted or unsubstituted C₆₋₃₀ iodoaryl, substituted or unsubstituted C₃₋₃₀ heteroaryl, substituted or unsubstituted C₇₋₂₀ arylalkyl, or substituted or unsubstituted C₄₋₂₀ heteroarylalkyl, or combinations thereof. Each of R¹⁷ to R¹⁹ may be either separate or connected to another group of R¹⁷ to R¹⁹ via a single bond or a divalent linking group to form a ring. R²⁰ and R²¹ may be either separate or connected to each other via a single bond or a divalent linking group to form a ring. Each of R¹⁷ to R²¹ optionally may include as part of its structure a divalent linking group. Each of R¹⁷ to R²¹ independently may optionally comprise an acid-labile group chosen, for example, from tertiary alkyl ester groups, secondary or tertiary aryl ester groups, secondary or tertiary ester groups having a combination of alkyl and aryl groups, tertiary alkoxy groups, acetal groups, or ketal groups.

Exemplary sulfonium cations of formula (8) may include one or more of the following:

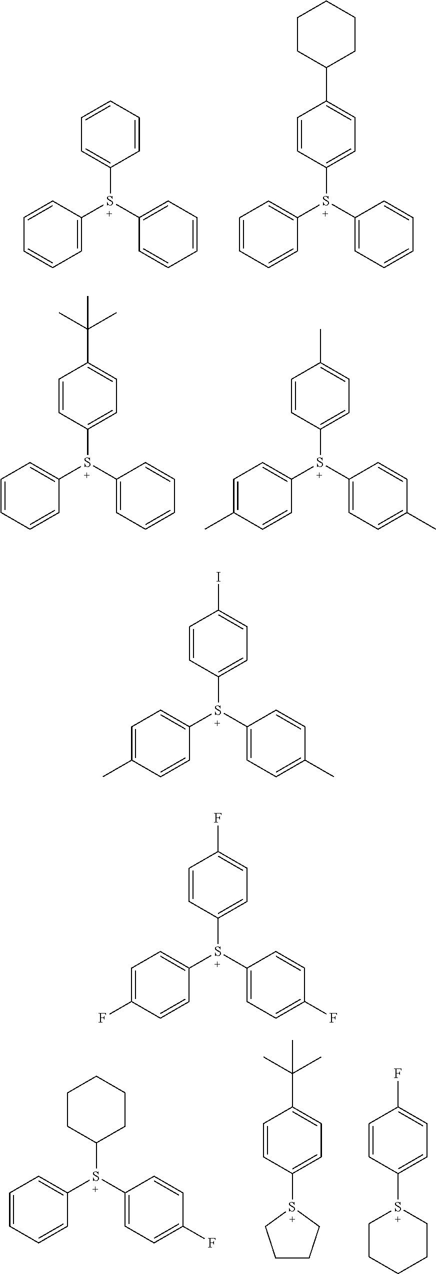

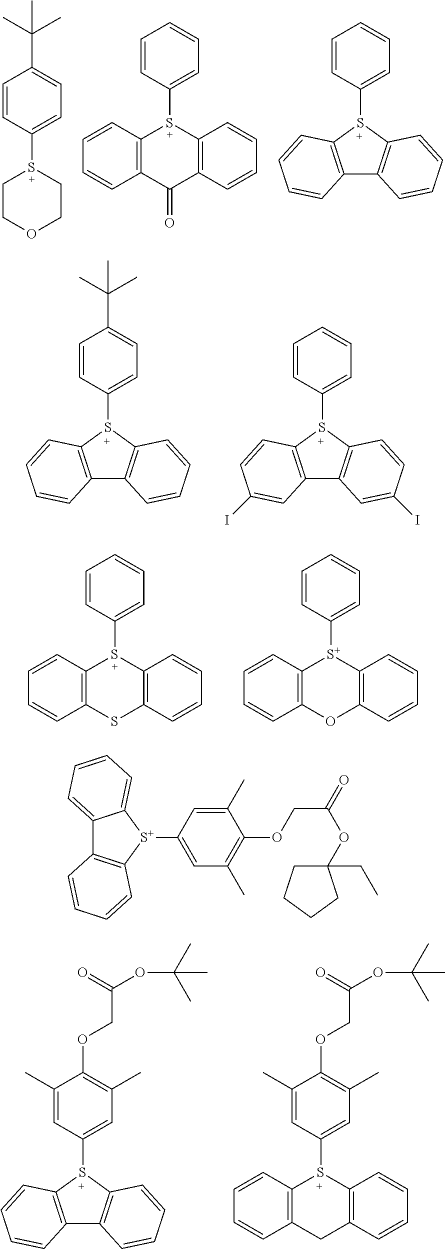

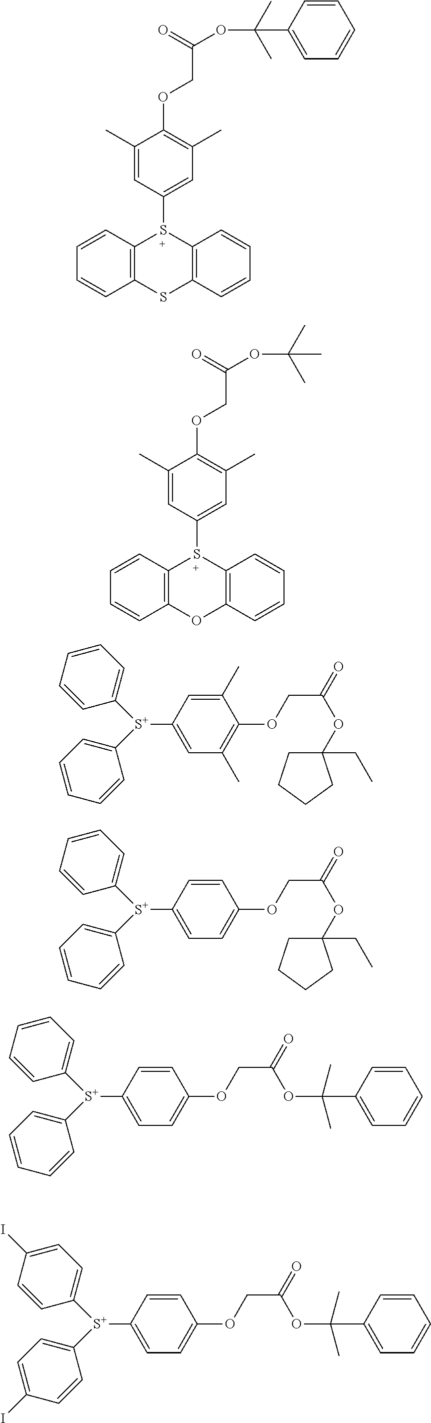

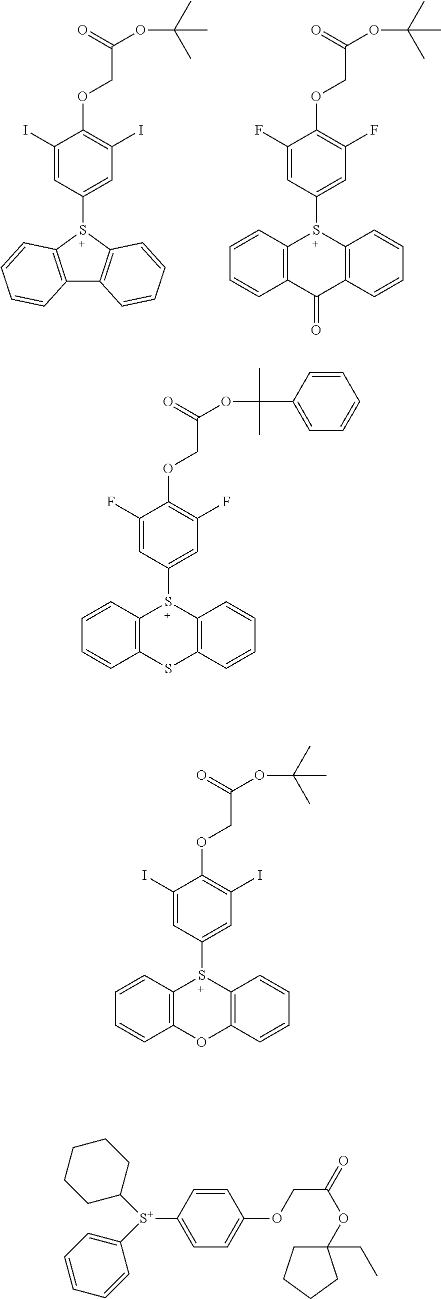

Exemplary iodonium cations of formula (9) may include one or more of the following:

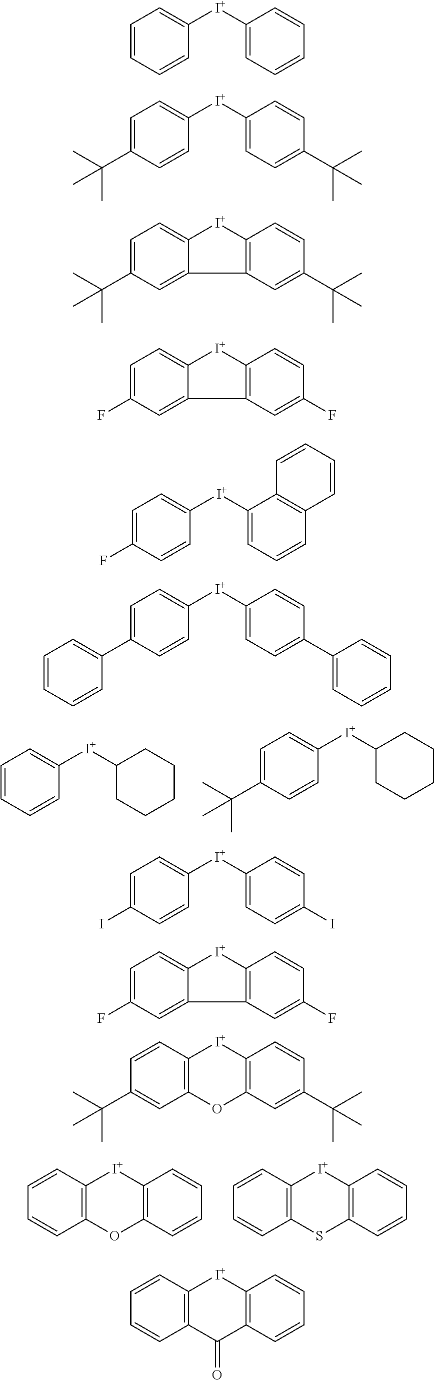

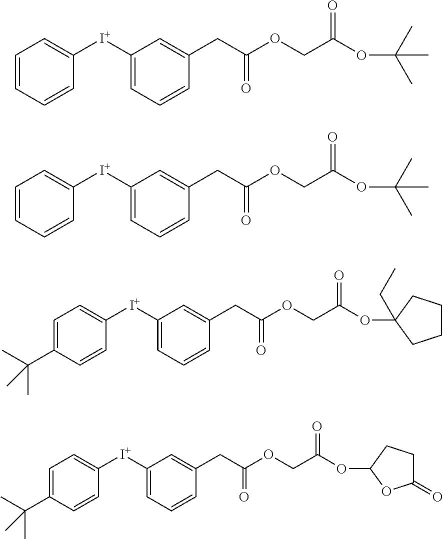

Exemplary organic anions having a sulfonate group may include one or more of the following:

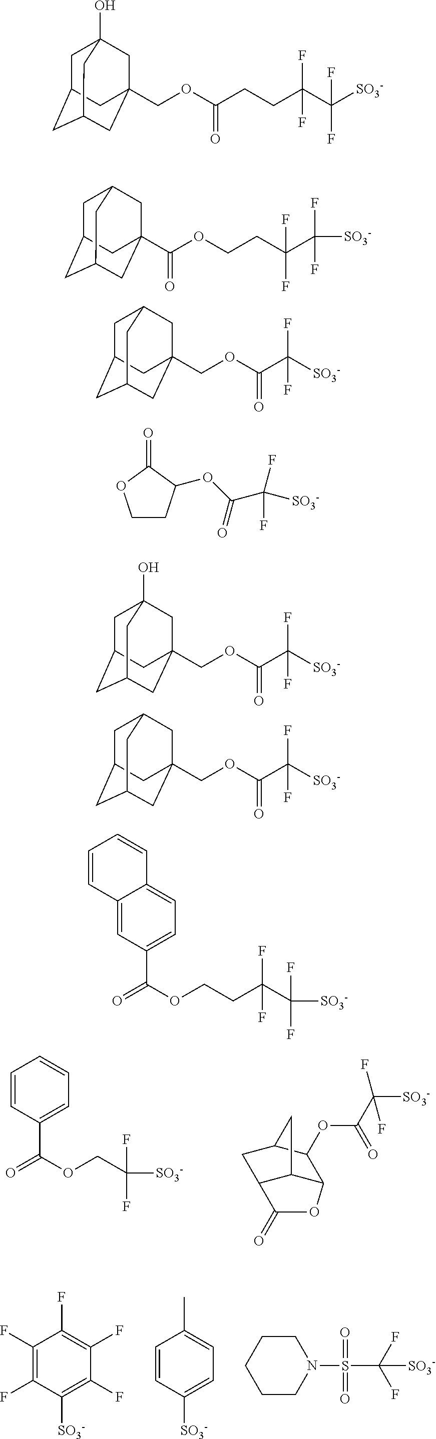

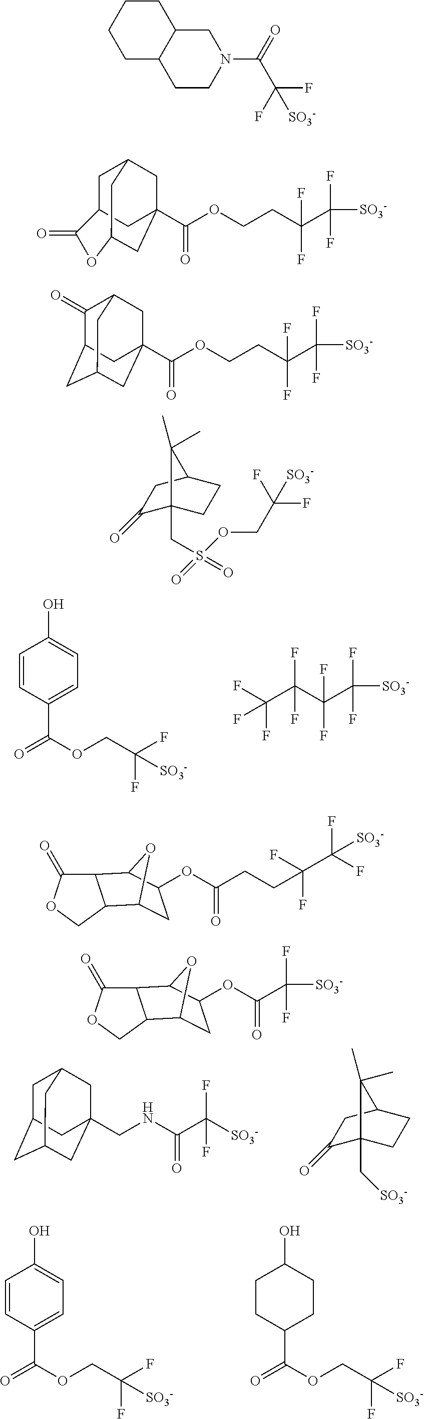

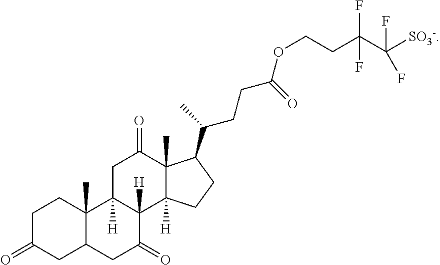

Exemplary non-sulfonated anions may include one or more of the following:

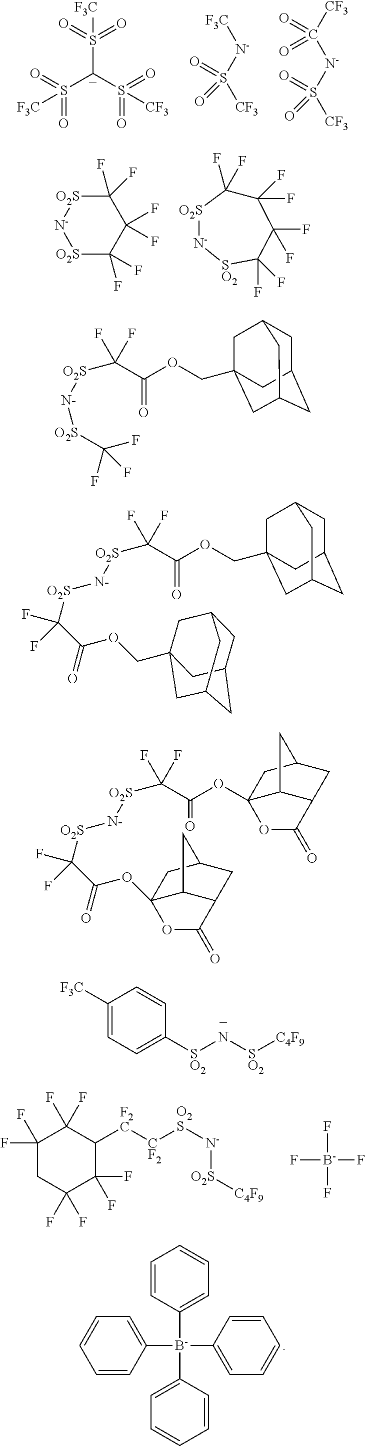

Commonly used onium salts may include, for example, triphenylsulfonium trifluoromethanesulfonate, (p-tert-butoxyphenyl)diphenylsulfonium trifluoromethanesulfonate, tris(p-tert-butoxyphenyl)sulfonium trifluoromethanesulfonate, triphenylsulfonium p-toluenesulfonate; di-t-butyphenyliodonium perfluorobutanesulfonate, and di-t-butyphenyliodonium camphorsulfonate. Other useful PAG compounds are known in the art of chemically amplified photoresists and include, for example: non-ionic sulfonyl compounds, for example, 2-nitrobenzyl-p-toluenesulfonate, 2,6-dinitrobenzyl-p-toluenesulfonate, and 2,4-dinitrobenzyl-p-toluenesulfonate; sulfonic acid esters, for example, 1,2,3-tris(methanesulfonyloxy)benzene, 1,2,3-tris(trifluoromethanesulfonyloxy)benzene, and 1,2.,-tris(p-toluenesulfonyloxy)benzene; diazomethane derivatives, for example, bis(benzenesulfonyl)diazomethane, bis(p-toluenesulfonyl)diazomethane; glyoxime derivatives, for example, bis-O-(p-toluenesulfonyl)-α-dimethylglyoxime, and bis-O-(n-butanesulfonyl)-α-dimethylglyoxime; sulfonic acid ester derivatives of an N-hydroxyimide compound, for example, N-hydroxysuccinimide methanesulfonic acid ester, N-hydroxysuccinimide trifluoromethanesulfonic acid ester; and halogen-containing triazine compounds, for example, 2-(4-methoxyphenyl)-4,6-bis(trichloromethyl)-1,3,5-triazine, and 2-(4-methoxynaphthyl)-4,6-bis (trichloromethyl)-1,3,5-triazine. Suitable photoacid generators are further described in U.S. Pat. Nos. 8,431,325 and 4.189.323.

The photoresist compositions may further include a basic quencher and/or a photo-decomposable quencher (PDQ) (also known as a photo-decomposable base). If used, the content of each is typically from 0.01 to 10 wt %, based on total solids of the photoresist composition.

Exemplary basic quenchers include, for example: linear aliphatic amines such as tributylamine, trioctylamine, triisopropanolamine, tetrakis (2-hydroxypropyl) ethylenediamine; n-tert-butyldiethanolamine, tris(2-acetoxy-ethyl)amine, 2,2′,2″,2′″-(ethane-1,2-diylbis(azanetriyl))tetraethanol, 2-(dibutylamino)ethanol, and 2,2′,2″-nitrilotriethanol; cyclic aliphatic amines such as 1-(tert-butoxycarbonyl)-4-hydroxypiperidine, tert-butyl 1-pyrrolidinecarboxylate, tert-butyl 2-ethyl-1H-imidazole-1-carboxylate, di-tert-butyl piperazine-1,4-dicarboxylate, and N-(2-acetoxy-ethyl) morpholine; aromatic amines such as pyridine, di-tert-butyl pyridine, and pyridinium; linear and cyclic amides and derivatives thereof such as N,N-bis(2-hydroxyethyl)pivalamide, N,N-diethylacetamide, N1,N1,N3,N3-tetrabutylmalonamide, 1-methylazepan-2-one, 1-allylazepan-2-one, and tert-butyl 1,3-dihydroxy-2-(hydroxymethyl)propan-2-vlcarbamate; ammonium salts such as quaternary ammonium salts of sulfonates, sulfamates, carboxylates, and phosphonates; imines such as primary and secondary aldimines and ketimines; diazines such as optionally substituted pyrazine, piperazine, and phenazine; diazoles such as optionally substituted pyrazole, thiadiazole, and imidazole; and optionally substituted pyrrolidones such as 2-pyrrolidone and cyclohexyl pyrrolidine.

Photo-decomposable quenchers PDQs generate a relatively weak acid upon irradiation that does not react rapidly with acid-labile groups in the photoresist composition. Exemplary photo-decomposable quenchers include, for example, photo-decomposable cations, and preferably those also useful for preparing strong acid generators but paired with an anion of a weak acid such as, for example, a C₁₋₂₀ carboxylic acid or C₁₋₂₀ sulfonic acid. Exemplary carboxylic acids include formic acid, acetic acid, propionic acid, tartaric acid, succinic acid, cyclohexanecarboxylic acid, benzoic acid, salicylic acid, and the like. Exemplary carboxylic acids include p-toluene sulfonic acid, camphor sulfonic acid and the like. In a preferred embodiment, the photo-decomposable quencher is a photo-decomposable organic zwitterion compound such as diphenyliodonium-2-carboxylate.

The photoresist composition may further include a material that comprises one or more base-labile groups (a “base-labile material”). As referred to herein, base-labile groups are functional groups that can undergo cleavage reaction to provide polar groups such as hydroxyl, carboxylic acid, sulfonic acid, and the like, in the presence of an aqueous alkaline developer after exposure and post-exposure baking steps. The base-labile group will not react significantly (e.g., will not undergo a bond-breaking reaction) prior to a development step of the photoresist composition that comprises the base-labile group. Thus, for instance, a base-labile group will be substantially inert during pre-exposure soft-bake, exposure, and post-exposure bake steps. By “substantially inert” it is meant that ≤5%, preferably ≤1%, of the base-labile groups (or moieties) will decompose, cleave, or react during the pre-exposure soft-bake, exposure, and post-exposure bake steps. The base-labile group is reactive under typical photoresist development conditions using, for example, an aqueous alkaline photoresist developer such as a 0.26 normal (N) aqueous solution of tetramethylammonium hydroxide (TMAH). For example, a 0.26 N aqueous solution of TMAH may be used to develop the resist pattern using a single puddle development or dynamic development process, e.g., where the 0.26 N TMAH developer is dispensed onto an imaged photoresist layer for a suitable time such as 10 to 120 seconds. An exemplary base-labile group is an ester group, typically a fluorinated ester group. Preferably, the base-labile material is substantially not miscible with and has a lower surface energy than other solid components of the photoresist composition. When coated on a substrate, the base-labile material can thereby segregate from other solid components of the photoresist composition to the top surface of the formed photoresist layer.

The base-labile material is preferably a polymeric material, also referred to herein as a base-labile polymer, which may include one or more repeating units comprising one or more base-labile groups. For example, the base-labile polymer may comprise a repeating unit comprising 2 or more base-labile groups that are the same or different. A preferred base-labile polymer comprises at least one repeating unit comprising 2 or more base-labile groups, for example a repeating unit comprising 2 or 3 base-labile groups.

The base-labile polymer may be a polymer comprising a repeating unit derived from a monomer of formula (E1)

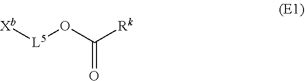

wherein X^{b} is a polymerizable group selected from vinyl and acrylic, L⁵ is a divalent linking group comprising one or more of substituted or unsubstituted linear or branched C₁₋₂₀ alkylene, substituted or unsubstituted C₃₋₂₀ cycloalkylene, —C(O)—, or —C(O)O—; and R^{k} is a substituted or unsubstituted C₁₋₂₀ fluoroalkyl group provided that the carbon atom bonded to the carbonyl (C═O) in formula (E1) is substituted with at least one fluorine atom.

Exemplary monomers of formula (E1) include the following:

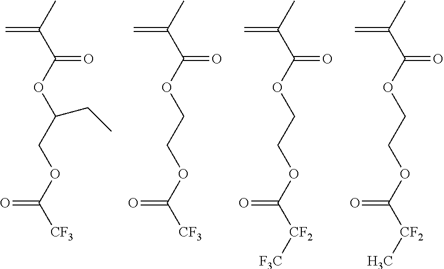

The base-labile polymer may include a repeating unit including two or more base-labile groups. For example, the base-labile polymer can include a repeating unit derived from a monomer of formula (E2):

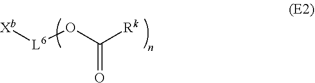

wherein X^{b} and R^{k} are as defined in formula (E1); L⁶ is a polyvalent linking group comprising one or more of substituted or unsubstituted straight chain or branched C₁₋₂₀ alkylene, substituted or unsubstituted C₃₋₂₀ cycloalkylene, —C(O)—, or —C(O)O—; and n is an integer of 2 or more, for example, 2 or 3.

Exemplary monomers of formula (E2) include the following:

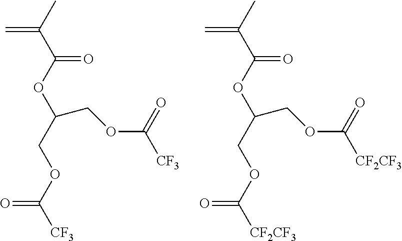

The base-labile polymer may include a repeating unit including one or more base-labile groups. For example, the base-labile polymer can include a repeating unit derived from a monomer of formula (E3):

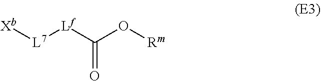

wherein X^{b} is as defined in formula (E1); L⁷ is a divalent linking group comprising one or more of substituted or unsubstituted straight chain or branched C₁₋₂₀ alkylene, substituted or unsubstituted C₃₋₂₀ cycloalkylene, —C(O)—, or —C(O)O—; L^{f} is a substituted or unsubstituted C₁₋₂₀ fluoroalkylene group wherein the carbon atom bonded to the carbonyl (C═O) in formula (E1) is substituted with at least one fluorine atom; and R^{m} is substituted or unsubstituted straight chain or branched C₁₋₂₀ alkyl, or substituted or unsubstituted C₃₋₂₀ cycloalkyl.

Exemplary monomers of formula (E3) include the following:

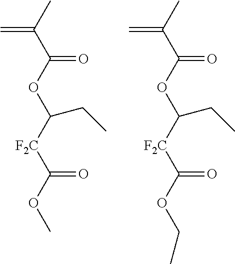 [SMILES: C=C(C)C(=O)OC(CC)C(F)(F)C(=O)OC ; C=C(C)C(=O)OC(CC)C(F)(F)C(=O)OCC]

In a further aspect of the invention, a base-labile polymer may comprise one or more base-labile groups and one or more acid-labile groups, such as one or more acid-labile ester moieties (e.g., t-butyl ester) or acid-labile acetal groups. For example, the base-labile polymer may comprise a repeating unit including a base-labile group and an acid-labile group, i.e., wherein both a base-labile group and an acid-labile group are present on the same repeating unit. In another example, the base-labile polymer may comprise a first repeating unit comprising a base-labile group and a second repeating unit comprising an acid-labile group. Preferred photoresists of the invention can exhibit reduced defects associated with a resist relief image formed from the photoresist composition. If present, the content of the base-labile polymer is typically from 0.01 to 10 wt %, based on total solids of the photoresist composition.

The base-labile polymer may be prepared using any suitable methods in the art, including those described herein for the first and second polymers. For example, the base-labile polymer may be obtained by polymerization of the respective monomers under any suitable conditions, such as by heating at an effective temperature, irradiation with actinic radiation at an effective wavelength, or a combination thereof. Additionally, or alternatively, one or more base-labile groups may be grafted onto the backbone of a polymer using suitable methods.

The photoresist composition may further include one or more additional optional additives. For example, optional additives may include actinic and contrast dyes, anti-striation agents, plasticizers, speed enhancers. sensitizers. surfactants, and the like, or combinations thereof. If present. the content of each optional additive is typically from 0.01 to 10 wt %. based on total solids of the photoresist composition.

Exemplary surfactants include fluorinated and non-fluorinated surfactants and can be ionic or non-ionic, with non-ionic surfactants being preferable. Exemplary fluorinated non-ionic surfactants include perfluoro C₄ surfactants such as FC-4430 and FC-4432 surfactants (3M Corporation); and fluorodiols such as POLYFOX PF-636. PF-6320. PF-656. and PF-6520 fluorosurfactants (Omnova).

The photoresist compositions further include a solvent for dissolving the components of the composition and facilitating its coating on a substrate. Preferably, the solvent is an organic solvent conventionally used in the manufacture of electronic devices. Suitable solvents include, for example: aliphatic hydrocarbons such as hexane and heptane; aromatic hydrocarbons such as toluene and xylene; halogenated hydrocarbons such as dichloromethane, 1,2-dichloroethane and 1-chlorohexane; alcohols such as methanol, ethanol, 1-propanol, iso-propanol. tert-butanol. 2-methyl-2-butanol, 4-methyl-2-pentanol, and diacetone alcohol (4-hydroxy-4-methyl-2-pentanone) (DAA); propylene glycol monomethyl ether (PGME); ethers such as diethyl ether, tetrahydrofuran, 1,4-dioxane and anisole; ketones such as acetone, methyl ethyl ketone, methyl iso-butyl ketone, 2-heptanone and cyclohexanone (CHO); esters such as ethyl acetate, n-butyl acetate, propylene glycol monomethyl ether acetate (PGMEA), ethyl lactate (EL), hydroxyisobutyrate methyl ester (HBM) and ethyl acetoacetate; lactones such as gamma-butyrolactone (GBL) and epsilon-caprolactone; lactams such as N-methyl pyrrolidone; nitriles such as acetonitrile and propionitrile; cyclic or non-cyclic carbonate esters such as propylene carbonate, dimethyl carbonate, ethylene carbonate, propylene carbonate, diphenyl carbonate, and propylene carbonate; polar aprotic solvents such as dimethyl sulfoxide and dimethyl formamide; water; and combinations thereof. Of these, preferred solvents are PGME, PGMEA, EL, GBL, HBM, CHO, DAA and combinations thereof. The solvent is typically organic-based, meaning the cumulative content of organic solvents (less impurities) in the solvent is 50 wt % or more, typically 95 wt % or more, 95 wt % or more, 98 wt % or more, or 100 wt %, based on total weight of the solvents. The total solvent content (i.c., cumulative solvent content for all solvents) in the photoresist compositions is typically from 40 to 99 wt %, for example, from 70 to 99 wt %, or from 85 to 99 wt %, based on the total photoresist composition. The desired solvent content will depend. for example, on the desired thickness of the coated photoresist layer and coating conditions.

The photoresist compositions can be prepared following known procedures. For example, the compositions can be prepared by dissolving solid components of the photoresist composition in the solvent. The photoresist compositions or one or more of the components of the compositions can optionally be subjected to one or more purification processes, for example, filtration and/or ion exchange processes. The desired total solids content of the compositions will depend on factors such as the desired final layer thickness. The solids content of the photoresist compositions is typically from 1 to 10 wt %, more preferably from 1 to 5 wt %, based on the total weight of the composition.

### Pattern Formation Methods

Patterning methods using the photoresist compositions of the invention are described below. Suitable substrates on which the photoresist compositions can be coated include electronic device substrates. A wide variety of electronic device substrates may be used in the present invention, such as: semiconductor wafers; polycrystalline silicon substrates; packaging substrates such as multichip modules; flat panel display substrates; substrates for light emitting diodes (LEDs) including organic light emitting diodes (OLEDs); and the like, with semiconductor wafers being typical. Such substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. Suitable substrates may be in the form of wafers such as those used in the manufacture of integrated circuits, optical sensors, flat panel displays, integrated optical circuits, and LEDs. Such substrates may be any suitable size. Typical wafer substrate diameters are 200 to 300 millimeters (mm), although wafers having smaller and larger diameters may be suitably employed according to the present invention. The substrates may include one or more layers or structures which may optionally include active or operable portions of devices being formed.

Typically, one or more lithographic layers such as a hardmask layer, for example, a spin-on-carbon (SOC), amorphous carbon, or metal hardmask layer, a CVD layer such as a silicon nitride (SiN), a silicon oxide (SiO), or silicon oxynitride (SiON) layer, an organic or inorganic underlayer, or combinations thereof, are provided on an upper surface of the substrate prior to coating a photoresist composition of the present invention. Such layers, together with an overcoated photoresist layer, form a lithographic material stack.

Optionally, a layer of an adhesion promoter may be applied to the substrate surface prior to coating the photoresist compositions. If an adhesion promoter is desired, any suitable adhesion promoter for polymer films may be used, such as silanes, typically organosilanes such as trimethoxyvinylsilane, tricthoxyvinylsilane, hexamethyldisilazane, or an aminosilane coupler such as gamma-aminopropyltriethoxysilane. Particularly suitable adhesion promoters include those sold under the AP 3000, AP 8000, and AP 9000S designations, available from DuPont Electronics & Imaging (Marlborough, Massachusetts).

The photoresist composition may be coated on the substrate by any suitable method, including spin-coating, spray-coating, dip-coating, doctor-blading, or the like. For example, applying the layer of photoresist may be accomplished by spin-coating the photoresist in solvent using a coating track, in which the photoresist is dispensed on a spinning wafer. During dispensing, the wafer is typically spun at a speed of up to 4,000 rotations per minute (rpm), for example, from 200 to 3,000 rpm, for example, 1,000 to 2,500 rpm, for a period of from 15 to 120 seconds to obtain a layer of the photoresist composition on the substrate. It will be appreciated by those skilled in the art that the thickness of the coated layer may be adjusted by changing the spin speed and/or the solids content of the composition. A photoresist layer formed from the compositions of the invention typically has a dried layer thickness of from 10 to 200 nanometers (nm), preferably from 15 to 100 nm, and more preferably from 20 to 60 nm

The photoresist composition is typically next soft-baked to minimize the solvent content in the layer, thereby forming a tack-free coating and improving adhesion of the layer to the substrate. The soft bake is performed, for example, on a hotplate or in an oven, with a hotplate being typical. The soft bake temperature and time will depend, for example, on the particular photoresist composition and thickness. The soft bake temperature is typically from 90 to 170° C., for example, from 110 to 150° C. The soft bake time is typically from 10 seconds to 20 minutes, for example, from 1 minute to 10 minutes, or from 1 minute to 5 minutes. The heating time can be readily determined by one of ordinary skill in the art based on the ingredients of the composition.

The photoresist layer is next pattern-wise exposed to activating radiation to create a difference in solubility between exposed and unexposed regions. Reference herein to exposing a photoresist composition to radiation that is activating for the composition indicates that the radiation is capable of forming a latent image in the photoresist composition. The exposure is typically conducted through a patterned photomask that has optically transparent and optically opaque regions corresponding to regions of the resist layer to be exposed and unexposed, respectively. Such exposure may, alternatively, be conducted without a photomask in a direct writing method, typically used for e-beam lithography. The activating radiation is typically sub-400 nm, sub-300 nm or sub-200 nm, with 248 nm (KrF) or 13.5 nm (EUV) wavelength or e-beam lithography being preferred. Of these, 13.5 nm (EUV) is particularly preferred. The exposure energy is typically from 1 to 200 millijoules per square centimeter (mJ/cm²), preferably 10 to 100 mJ/cm² and more preferably 20 to 50 mJ/cm², dependent upon the exposure tool and components of the photoresist composition. In some aspects, the activating radiation is EUV at a wavelength of 13.5 nm.

Following exposure of the photoresist layer, a post-exposure bake (PEB) of the exposed

photoresist layer is performed. The PEB can be conducted, for example, on a hotplate or in an oven, with a hotplate being typical. Conditions for the PEB will depend, for example, on the particular photoresist composition and layer thickness. The PEB is typically conducted at a temperature of from 80 to 150° C., and a time of from 30 to 120 seconds. A latent image defined by the polarity-switched (exposed regions) and unswitched regions (unexposed regions) is formed in the photoresist.

The exposed photoresist layer is then developed with a suitable developer to selectively remove those regions of the layer that are soluble in the developer while the remaining insoluble regions form the resulting photoresist pattern relief image. In the case of a positive-tone development (PTD) process. the exposed regions of the photoresist layer are removed during development and unexposed regions remain. Conversely, in a negative-tone development (NTD) process, the exposed regions of the photoresist layer remain, and unexposed regions are removed during development. Application of the developer may be accomplished by any suitable method such as described above with respect to application of the photoresist composition, with spin coating being typical. The development time is for a period effective to remove the soluble regions of the photoresist, with a time of from 5 to 60 seconds being typical. Development is typically conducted at room temperature.

Suitable developers for a PTD process include aqueous base developers, for example, quaternary ammonium hydroxide solutions such as tetramethylammonium hydroxide (TMAH), preferably 0.26 normal (N) TMAH, tetraethylammonium hydroxide, tetrabutylammonium hydroxide, sodium hydroxide, potassium hydroxide, sodium carbonate, potassium carbonate, and the like. Suitable developers for an NTD process are organic solvent-based, meaning the cumulative content of organic solvents in the developer is 50 wt % or more, typically 95 wt % or more. 95 wt % or more, 98 wt % or more, or 100 wt %, based on total weight of the developer. Suitable organic solvents for the NTD developer include, for example, those chosen from ketones, esters, ethers, hydrocarbons, and mixtures thereof. The NTD developer is typically n-butyl acetate or 2-heptanone.

A coated substrate may be formed from the photoresist compositions of the invention. Such a coated substrate includes: (a) a substrate having one or more layers on a surface thereof; and (b) a layer of the photoresist composition over the one or more layers.

The photoresist pattern may be used, for example, as an etch mask, thereby allowing the pattern to be transferred to one or more sequentially underlying layers by known etching techniques, typically by dry-etching such as reactive ion etching. The photoresist pattern may, for example, be used for pattern transfer to an underlying hardmask layer which, in turn, is used as an etch mask for pattern transfer to one or more layers below the hardmask layer. If the photoresist pattern is not consumed during pattern transfer, it may be removed from the substrate by known techniques, for example, oxygen plasma ashing or a wet strip process. The photoresist compositions may, when used in one or more such patterning processes, be used to fabricate semiconductor devices such as memory devices, processor chips (CPUs), graphics chips, optoclectronic chips, LEDs, OLEDs, as well as other electronic devices.

The following non-limiting examples are illustrative of the invention.

### EXAMPLES

### Enol Ether Compound Synthesis

### Example 1 (Compound SM1)

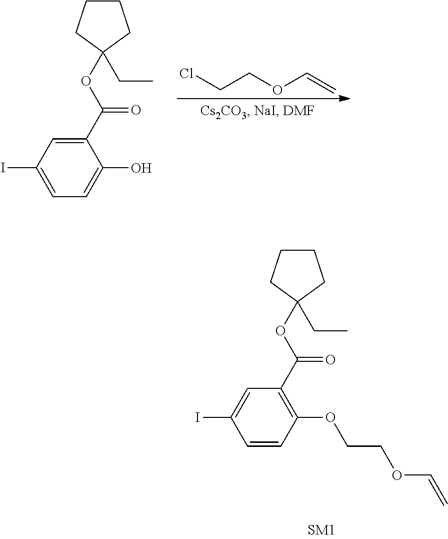

In a reaction vessel, 1-ethylcyclopentyl-2-hydroxy-5-iodobenzoate (36.0 g, 100 mmol) was suspended in DMF to achieve a 9 wt % solution. Cesium carbonate (65.1 g, 200 mmol) and sodium iodide (1.50 g, 10 mmol) were added neatly to the solution to form a reaction mixture. (2-chloroethoxy) ethene (12.8 g, 120 mmol) was next added slowly and dropwise to the reaction mixture while stirring. The reaction mixture was heated to an internal temperature of 70° C. for 4 hours. Then, additional (2-chloroethoxy) ethene (10.7 g, 100 mmol) was added to the reaction mixture, and the reaction mixture was heated at 70° C. for an additional 14 hours. The reaction mixture was allowed to cool to room temperature and then was diluted with DI water (1.5 L), and the aqueous mixture was extracted with methyl tert-butyl ether (3×250 mL). The organic layers were combined and washed with water (5×200 mL), and the solvents were removed under a reduced pressure to afford enol ether compound SM1 as a colorless oil (37.5 g, yield of 87%). [¹H-NMR (500 MHz, chloroform-d) δ (ppm): 7.94 (d, 1H, ArH), 7.66 (dd, 1H, ArH), 6.73 (d, 1H, ArH), 6.51 (dd, 1H, alkenyl-H), 4.27-4.18 (m, 3H, CH₂/alkenyl-H), 4.08-4.00 (m, 3H, CH₂/alkenyl-H), 2.29-2.18 (m, 2H, CH₂), 2.09 (q, 2H, CH₂), 1.82-1.68 (m, 4H, CH₂), 1.67-1.57 (m, 2H, CH₂), 0.93 (t, 3H, CH₃).]

### Example 2 (Compound SM2)

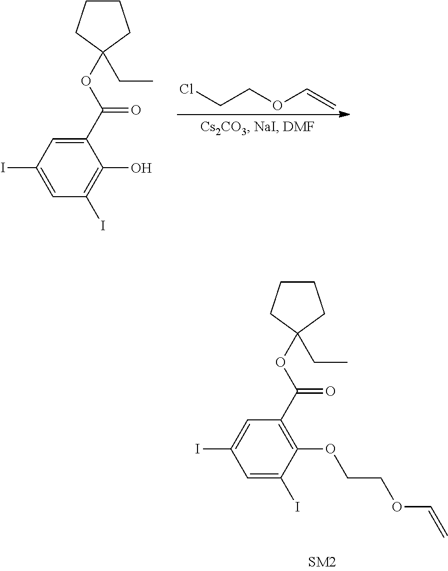

In a reaction vessel, 1-ethylcyclopentyl-2-hydroxy-3,5-diiodobenzoate (36.5 g, 75 mmol) was suspended in DMF to achieve a 12 wt % solution. Cesium carbonate (48.9 g, 150 mmol) and sodium iodide (1.12 g, 7.5 mmol) were added neatly to form a reaction mixture. (2-chloroethoxy) ethene (12.8 g, 120 mmol) was next added slowly added dropwise to the reaction mixture while stirring. The reaction mixture was heated to an internal temperature of 70° C. for 16 hours. Then, additional (2-chloroethoxy)ethene (9.6 g, 100 mmol) was added to the reaction mixture, and the reaction temperature was increased to 85° C. for 1 hour. The reaction mixture was allowed to cool to room temperature and was then diluted with DI water (1.5 L), and the aqueous mixture was extracted with ethyl acetate (4×200 mL). The organic layers were combined and washed with DI water (5×200 mL), and the solvents were removed under a reduced pressure to afford enol ether compound SM2 as a colorless oil (37.5 g, yield of 90%). [¹H-NMR (500 MHz, chloroform-d) δ (ppm): 8.20 (d, 1H, ArH), 7.92 (d, 1H, ArH), 6.52 (dd, 1H, alkenyl-H), 4.30-4.20 (m, 3H, CH₂/alkenyl-H), 4.10 (t, 2H, CH₂), 4.05 (dd, 1H, alkenyl-H), 2.28-2.15 (m, 2H, CH₂), 2.10 (q, 2H, CH₂), 1.84-1.71 (m, 4H, CH₂), 1.70-1.60 (m, 2H, CH₂), 0.93 (t, 3H, CH₃).]

### Example 3 (Compound SM3)

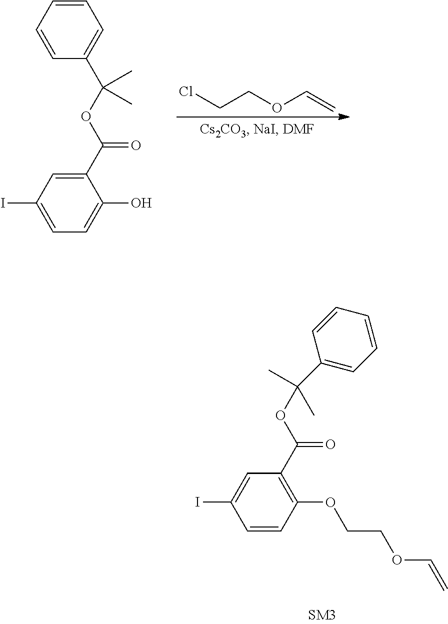

In a reaction vessel, 2-phenylpropan-2-yl-2-hydroxy-5-iodobenzoate (11.5 g, 30 mmol) was suspended in DMP to achieve a 11.5 wt % solution. Cesium carbonate (19.5 g, 60 mmol) and sodium iodide (0.5 g, 3 mmol) were added neatly to the solution to form a reaction mixture. (2-chloroethoxy)ethene (3.8 g, 36 mmol) was next added slowly and dropwise to the reaction mixture while stirring. The reaction mixture was heated to an internal temperature of 70° C. for 4 hours. Then, additional (2-chloroethoxy) ethene (3.2 g, 30 mmol) was added to the reaction mixture, and the reaction mixture was heated at 70° C. for an additional 16 hours. The reaction mixture was allowed to cool to room temperature and then diluted with DI water (1.0 L), and the aqueous mixture was extracted with ethyl acetate (3×100 mL). The organic layers were combined and washed with DI water (5×100 mL), and the solvents were removed under a reduced pressure to afford enol ether compound SM3 as a colorless oil (11.1 g, yield of 82%). [¹H-NMR (500 MHz, acetone-d₆) δ (ppm): 7.90 (s, 1H, ArH), 7.79 (d, 1H, ArH), 7.53 (d, 2H, ArH), 7.34 (t, 2H, ArH), 7.23 (t, 1H, ArH), 7.00 (d, 1H, ArH), 6.57 (dd, 1H, alkenyl-H), 4.34 (t, 2H, CH₂), 4.27 (d, 1H, alkenyl-H), 4.13 (t, 2H, CH₂), 4.01 (d, 1H, alkenyl-H), 1.86 (s, 6H, CH₃).]

### Acid-Sensitive Monomer Synthesis

### Example 4 (Monomer MC1)

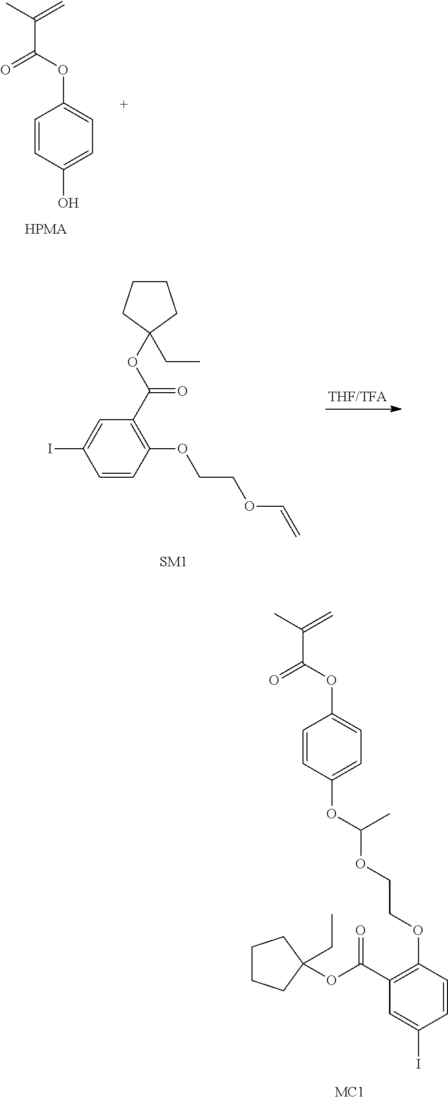

A reaction vessel is charged with monomer HPMA (5.0 g, 28.06 mmol) and anhydrous tetrahydrofuran 50 mL. Trifluoroacetic acid (0.03 g) and enol ether compound SM1 (12.0 g, 28.06 mmol) are added to the anhydrous solution to provide a reaction mixture. The reaction mixture is stirred at room temperature (ca. 23° C.) for 24 hours. The reaction mixture is then passed through an alumina column and the solvent is removed completely under reduced pressure. It is expected that monomer MC1 would result.

### Example 5 (Monomer MC2)

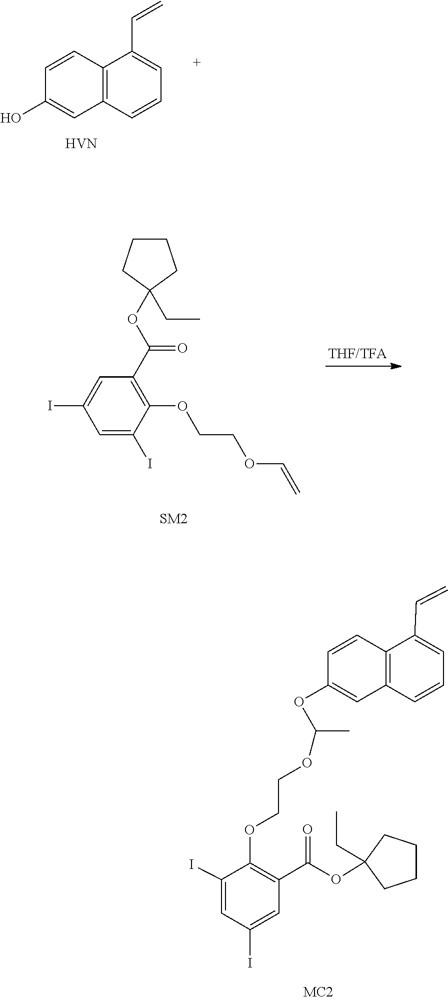

A reaction vessel is charged with monomer HVN (5.1 g, 30.0 mmol) and anhydrous tetrahydrofuran 75 mL. Trifluoroacetic acid (0.03 g) and enol ether compound SM2 (16.68.0 g, 30.0 mmol) are added to the anhydrous solution to provide a reaction mixture. The reaction mixture is stirred at room temperature (ca. 23° C.) for 24 hours. The reaction mixture is then passed through an alumina column and the solvent is removed completely under reduced pressure. It is expected that monomer MC2 would result.

### Example 6 (Monomer MC3)

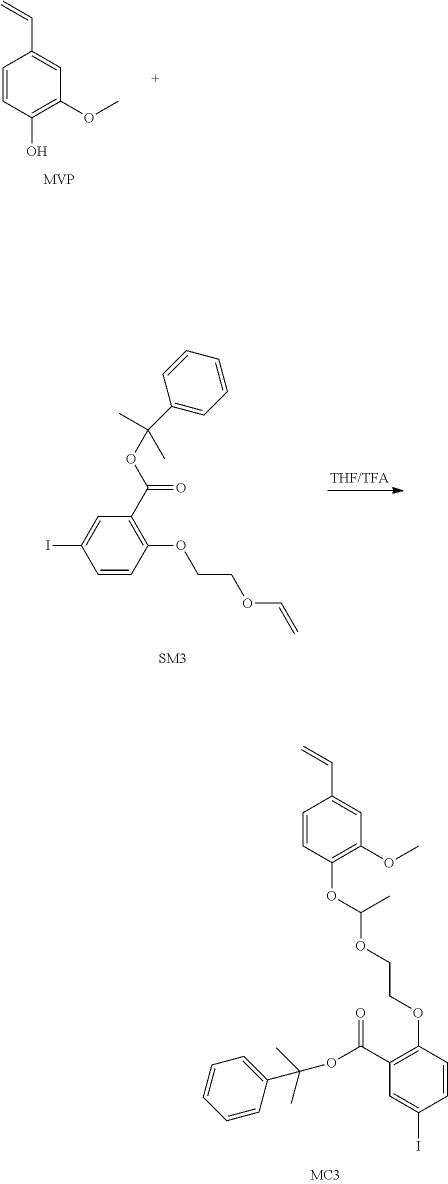 [SMILES: C=Cc1ccc(O)c(OC)c1 ; C=COCCOc1ccc(I)cc1C(=O)OC(C)(C)c1ccccc1 ; C=Cc1ccc(OC(C)OCCOc2ccc(I)cc2C(=O)OC(C)(C)c2ccccc2)c(OC)c1]

A reaction vessel is charged with monomer MVP (4.50 g, 30.0 mmol) and anhydrous tetrahydrofuran 60 mL. Trifluoroacetic acid (0.03 g) and enol ether compound SM1 (13.56 g, 30.0 mmol) are added to the anhydrous solution to provide a reaction mixture. The reaction mixture is stirred at room temperature (ca. 23° C.) for 24 hours. The reaction mixture is then passed through an alumina column and the solvent is removed completely under reduced pressure. It is expected that monomer MC3 would result.

### Polymer Synthesis

The following materials were used to prepare polymers as described below:

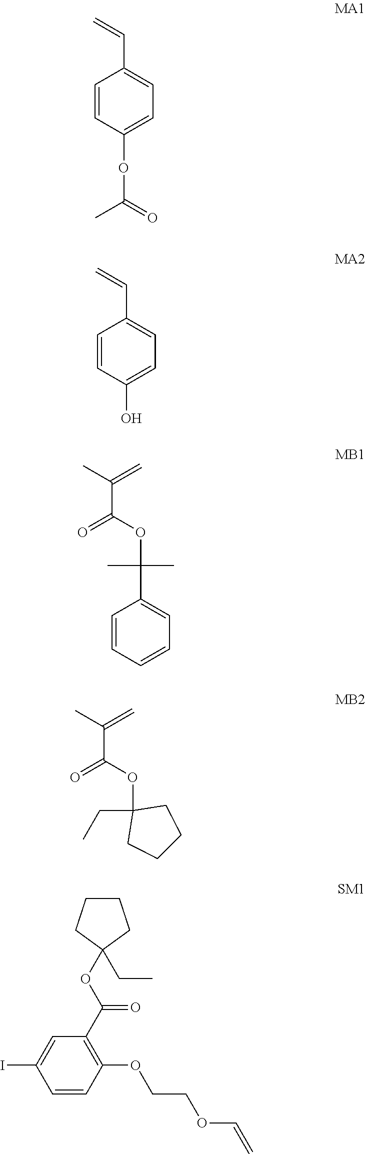

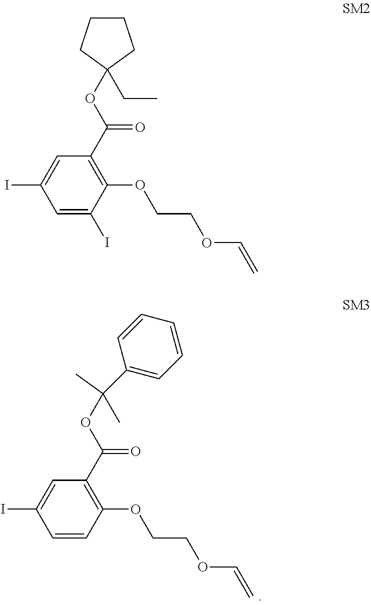 [SMILES: C=COCCOc1c(I)cc(I)cc1C(=O)OC1(CC)CCCC1 ; C=COCCOc1ccc(I)cc1C(=O)OC(C)(C)c1ccccc1]

### Example 7 (Polymer CP-1)

Polymer CP-1 was prepared from monomers MA-1 and MB-1, at a molar feed ratio of 50/50. A feed solution was made by dissolving monomer MA-1 (22.6 g, 140 mmol) and monomer MB-1 (28.5 g, 140 mmol) in 50 g of PGMEA. An initiator solution was separately prepared by dissolving 4.4 g of 2,2′-azobis (2,4-dimethylvaleronitrile) (obtained as V-65 from Wako Pure Chemical Industries, Ltd.) in 13 g PGMEA and THF (wt/wt 1/1). The polymerization was performed in a 3-neck round bottom flask fitted with a water condenser and a thermometer to monitor the reaction in the flask. The reactor was charged with 25 g of PGMEA and heated to 75° C. The feed solution and the initiator solution were each fed separately into the reactor using syringe pumps over 6 hours. Following the addition, the contents were then stirred for an additional 2 hours. The contents were subsequently cooled to room temperature and precipitated into 1 liter (L) of heptane. The resulting product was isolated by filtration and was dried overnight at 35° C. under vacuum. The product was then dissolved in methanol (500 mL) and combined with 30 wt % of sodium methoxide in a methanol solution (0.6 g). The reaction mixture was heated at 67° C. until completion of the reaction. The reaction mixture was allowed to cool to room temperature and was then neutralized with the addition of acidic ion exchange resin C381H (Evoqua Water Technologies LLC). The reaction converts the structure of the repeating unit derived from 4-acetoxystyrene (MA-1) to 4-hydroxystyrene (MA-2). The polymer solution was precipitated into DI water to isolate a white solid (about 36.3 g) [Mw=6.5 kilodaltons (kDa), PDI=1.63], which was further dried under vacuum at 35° C. Molecular weight was determined by gel permeation chromatography (GPC) using polystyrene standards.

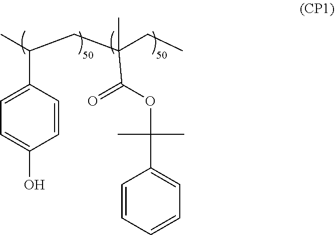

### Example 8 (Polymer CP-2)

Polymer CP-2 was prepared from the monomers MA-1 and MB-2, at a molar feed ratio of 50/50. A feed solution was made by dissolving monomer MA-1 (28.22 g, 174 mmol) and monomer MB-2 (31.78 g, 174 mmol) in 40 g of PGMEA. An initiator solution was separately prepared by dissolving 4.41 g of 2,2′-azobis (2,4-dimethylvaleronitrile) (obtained as V-65 from Wako Pure Chemical Industries, Ltd.) in 13.23 g PGMEA and THF (wt/wt 1/1). The polymerization was performed in a 3-neck round bottom flask fitted with a water condenser and a thermometer to monitor the reaction in the flask. The reactor was charged with 20 g of PGMEA and heated to 75° C. The feed solution and the initiator solution were each fed separately into the reactor using syringe pumps over 6 hours. Following the addition, the contents were then stirred for an additional 2 hours. The contents were subsequently cooled to room temperature and precipitated into 1 liter (L) of heptane. The resulting product was isolated by filtration and was dried overnight at 35° C. under vacuum. The product was then dissolved in methanol (600 mL) and combined with 30 wt % of sodium methoxide in a methanol solution (0.75 g). The reaction mixture was heated at 67° C. until completion of the reaction. The reaction mixture was allowed to cool to room temperature and was then neutralized with the addition of acidic ion exchange resin C381H (Evoqua Water Technologies LLC). The reaction converts the structure of the repeating unit derived from 4-acetoxystyrene (MA-1) to 4-hydroxystyrene (MA-2). The polymer solution was precipitated into DI water to isolate a white solid (about 42.2 g) [Mw=7.1 kilodaltons (kDa), PDI=1.74], which was further dried under vacuum at 35° C. Molecular weight was determined by gel permeation chromatography (GPC) using polystyrene standards.

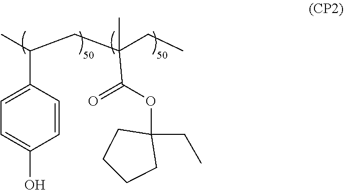

### Example 9 (Polymer P1)

A reaction vessel was charged with polymer CP1 (15.0 g) and PGMEA to provide a 20 wt % solution. Traces of water were removed by azeotropic distillation. Trifluoroacetic acid (0.014 g) and enol ether compound SM1 (4.38 g) were added to the anhydrous solution to provide a reaction mixture. The reaction mixture was stirred at room temperature (ca. 23° C.) for 24 hours. The reaction mixture was then passed through an alumina column and the solvent was removed under a reduced pressure to provide Polymer PI in PGMEA (48.8 g, 26.0 solid %, yield of 65.5%). Molecular weight was determined by gel permeation chromatography (GPC) using polystyrene standard. [Mw=7.9 kilodaltons (kDa), PDI=2.54].

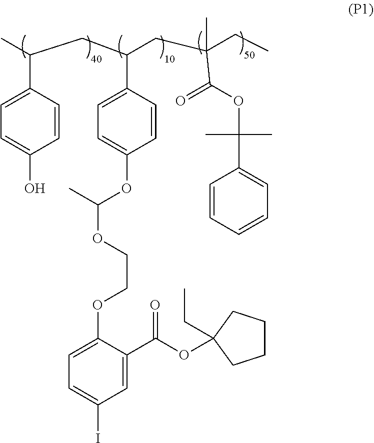

### Example 10 (Polymer P2)

A reaction vessel was charged with polymer CP-2 (15.2 g) and PGMEA to provide a 20 wt % solution. Traces of water were removed by azeotropic distillation. Trifluoroacetic acid (0.032 g) and enol ether compound SM2 (8.39 g) were added to the anhydrous solution to provide a reaction mixture. The reaction mixture was stirred at room temperature (ca. 23° C.) for 24 hours. The reaction mixture was then passed through an alumina column and the solvent was removed under a reduced pressure to provide Polymer P2 in PGMEA (84.1 g, 22.3 solid %, yield of 79.5%). Molecular weight was determined by gel permeation chromatography (GPC) using polystyrene standards. [Mw=8.9 kilodaltons (kDa), PDI=3.48].

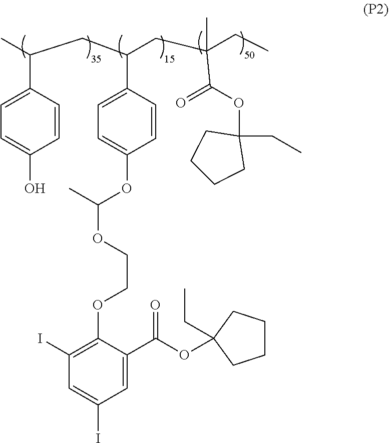

### Example 11 (Polymer P3)

A reaction vessel was charged with polymer CP-2 (14.1 g) and PGMEA to provide a 20 wt % solution. Traces of water were removed by azeotropic distillation. Trifluoroacetic acid (0.03 g) and enol ether compound SM3 (8.09 g) were added to the anhydrous solution to provide a reaction mixture. The reaction mixture was stirred at room temperature (ca. 23° C.) for 24 hours. The reaction mixture was then passed through an alumina column and the solvent was removed under a reduced pressure to provide Polymer P3 in PGMEA (86.5 g, 20.8 solid %, yield of 78.0%). (Mw=9.0 kilodaltons (kDa), PDI=3.49), which was further dried under vacuum at 35° C. Molecular weight was determined by gel permeation chromatography (GPC) using polystyrene standards.

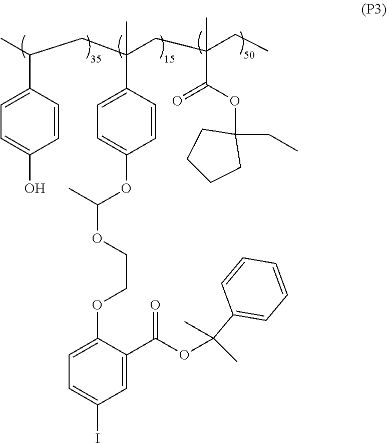

### Photoresist Composition Preparation

### Examples 12-16

Photoresist compositions were prepared by combining the components in the relative amounts shown in Table 1, where the amounts are expressed in weight percent (wt %) based on total solids. The photoresist compositions were each prepared in a solvent mixture of PGMEA and methyl-2-hydroxyisobutryate in a weight ratio of 1:1, with a resulting total solids content of 2.15 wt % based on the total composition. The compositions were shaken on a mechanical shaker and then filtered through a PTFE disk-shaped filter having a 0.2-micron pore size.

TABLE 1

| | Photoresist | | | | |
|---|---|---|---|---|---|
| Example | Composition | Polymer | PAG | Quencher | Solvent |
| 12 | PR-1 | P1 | PAG-1 | Q1 | S1/S2 |
| | | (78.74%) | (19.69%) | (1.57%) | (1:1 wt/wt) |
| 13 | PR-2 | P2 | PAG-1 | Q1 | S1/S2 |
| | | (78.74%) | (19.69%) | (1.57%) | (1:1 wt/wt) |
| 14 | PR-3 | P3 | PAG-1 | Q1 | S1/S2 |
| | | (78.74%) | (19.69%) | (1.57%) | (1:1 wt/wt) |
| 15 | CPR-1 | CP1 | PAG-1 | Q1 | S1/S2 |
| | | (78.74%) | (19.69%) | (1.57%) | (1:1 wt/wt) |
| 16 | CPR-2 | CP2 | PAG-1 | Q1 | S1/S2 |
| | | (78.74%) | (19.69%) | (1.57%) | (1:1 wt/wt) |

S1 = PGMEA;
S2 = methyl-2-hydroxyisobutryate

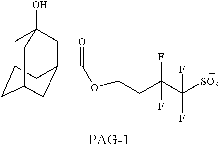

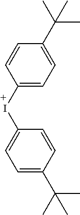

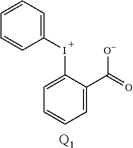

### Lithographic Evaluation Line Space (L/S) Patterning

### Examples 17-21

The photoresist compositions were evaluated for line/space (l/s) patterning under KrF exposure using a bright field mask pattern. 200 nm wafers for photolithographic testing were coated with ARTM3 organic antireflectant (DuPont Electronics & Industrial) and softbaked at 205° C. for 60 seconds to provide a 60 nm first BARC layer. A coating of ARTM40A organic antireflectant (DuPont Electronics & Industrial) was then disposed on the first BARC layer and softbaked at 215° C. for 60 seconds to provide a second BARC layer having a thickness of about 80 nm. A photoresist composition was then coated onto the dual BARC stack and soft-baked at 110° C. for 60 seconds to provide a photoresist film layer having a thickness of about 50 nm. The wafers were each exposed with 248 nm radiation on a Canon FPA-5000 ES4 scanner (NA=0.8, outer sigma=0.85, inner sigma=0.57) using a mask having 120 nm l/s patterns. The wafers were post-exposure baked at 100° C. for 60 seconds, developed with MF-CD26™ TMAH developer (DuPont Electronics & Industrial) for 60 seconds, rinsed with DI water, and dried. Critical dimension (CD) measurements of the formed l/s patterns were made with a Hitachi S-9380 CD-SEM. Sizing energy (Esize) and LWR (nm) of the lines were determined based on the CD measurements. Sizing energy is the irradiation energy at which the target 120 nm line-space pattern was resolved. The pseudo Z-factor is reported below and was determined according to Equation 1:

Pseudo Z-factor=(E_{size})×(LWR)² (Equation 1)

where E_{size} is reported in millijoules per square centimeter (mJ/cm²), LWR is reported in nanometers (nm), and the pseudo Z-factor is reported in mJ×10⁻¹¹. The pseudo Z-factor is a modified measure of photoresist performance based on the Z-factor, which is a known parameter indicative of RLS (Resolution, Line Width Roughness, Sensitivity) photoresist performance (see, e.g., Wallow, T. et al Proc. SPIE 6921, 69211F, 2008). The pseudo Z-factor is calculated at a constant resolution (CD size). The results are shown in Table 2.

TABLE 2

| | Photoresist | E_{size} | LWR | Pseudo-Z factor |
|---|---|---|---|---|
| Example | Composition | (mJ/cm²) | (nm) | (mJ E⁻¹¹) |
| 17 | PR-1 | 60.49 | 6.011 | 2.18 |
| 18 | PR-2 | 73.92 | 5.203 | 2.00 |
| 19 | PR-3 | 68.01 | 7.430 | 3.75 |
| 20 (Comp.) | CPR-1 | 56.66 | 9.313 | 4.91 |
| 21 (Comp.) | CPR-2 | 61.66 | 10.115 | 6.30 |

As shown in Table 2, the inventive photoresist compositions PR-1, PR-2, and PR-3 achieved improved (lower) LWR and improved (lower) pseudo-Z factor relative to comparative photoresist compositions CPR-1 and CPR-2.

### Trench (TR) Patterning

### Examples 22-26

The photoresist compositions were evaluated for trench (TR) patterning under KrF exposure as described in Examples 17-21 and using a dark field mask pattern. Esize and LWR of the spaces were determined based on the CD measurements. Sizing energy was determined from the irradiation energy at which the target 120 nm trench pattern was resolved. The results are shown in Table 3.

TABLE 3

| | Photoresist | E_{size} | LWR | Pseudo-Z factor |
|---|---|---|---|---|
| Example | Composition | (mJ/cm²) | (nm) | (mJ E⁻¹¹) |
| 22 | PR-1 | 64.652 | 6.205 | 2.48 |
| 23 | PR-2 | 86.63 | 5.234 | 2.37 |
| 24 | PR-3 | 78.00 | 4.962 | 1.92 |
| 25 (Comp) | CPR-1 | 57.769 | 9.339 | 5.03 |
| 26 (Comp) | CPR-2 | 60.308 | 9.317 | 5.23 |

As shown in Table 3, the inventive photoresist compositions PR-1B, PR-2B, and PR-3B achieved improved (lower) LWR and improved (lower) pseudo-Z factor relative to the comparative photoresist compositions CPR-1B and CPR-2B.

1. A compound comprising a C₆₋₃₀ aromatic or C₃₋₃₀ heteroaromatic core, wherein the core comprises a first substituent comprising (i) an enol ether group or (ii) a halomethyl ether group, wherein the halomethyl ether group is substituted or unsubstituted, a second substituent comprising an acid labile group, and a third substituent that is a halogen atom, wherein the core is optionally further substituted.

2. The compound of claim 1, wherein the compound is represented by formula (1) or (2): 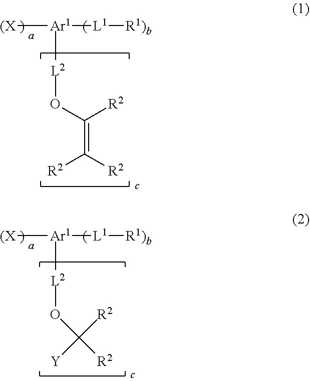
wherein:
Ar¹ is a C₆₋₃₀ aromatic or C₃₋₃₀ heteroaromatic core;
R¹ independently represents a moiety comprising an acid labile group;
each R² independently represents H, substituted or unsubstituted C₁₋₁₀ alkyl, substituted or unsubstituted C₁₋₁₀ haloalkyl, optionally including as part of its structure one or more groups chosen from —O—, —S—, —N(R³)—, —C(O)—, —C(O)O—, or —C(O)N(R³)—, wherein R³ represents H, substituted or unsubstituted C₁₋₁₀ alkyl, substituted or unsubstituted C₁₋₁₀ heteroalkyl, substituted or unsubstituted C₆₋₃₀ aryl, or substituted or unsubstituted C₄₋₃₀ heteroaryl, any two R² groups in formula (1) or the two R² groups in formula (2) together optionally forming a ring, and one or more R² groups and L² together optionally forming a ring;
X independently represents a halogen atom;
Y independently represents Cl, Br, or I;
each L¹and L² independently represents a single bond or a divalent linking group; and
a, b, and c independently represent an integer greater than or equal to 1;
wherein Ar¹ is optionally further substituted.

3. The compound of claim 2, wherein X is a fluorine atom or an iodine atom.

4. The compound of claim 2, wherein R¹ comprises an acid-labile ester group, an acetal group, or a ketal group.

5. The compound of claim 2, wherein c is 1.

6. The compound of claim 2, wherein c is 2.

7. The compound of claim 2, wherein L² comprises an —S(O)₂— group.

8. A monomer, comprising the reaction product of (i) a compound of claim 1, and (ii) a monomer precursor comprising a polymerizable group and a group chosen from an —OH group or a —C(O)OH group, wherein the reaction product comprises an acetal or ketal linker formed by reaction between an enol ether moiety or a halomethyl ether moiety of the compound and the —OH or —C(O)OH group of the monomer precursor.

9. The monomer of claim 8, wherein the monomer is of the following general formula (4): 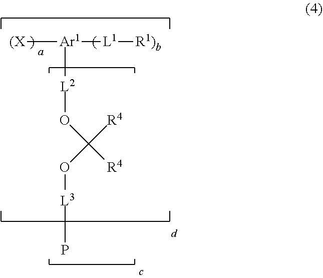
wherein:
P is a polymerizable group;
Ar¹ is a C₆₋₃₀ aromatic or C₃₋₃₀ heteroaromatic core;
R¹ independently represents a moiety comprising an acid labile group;
R⁴ independently represents H, substituted or unsubstituted C₁₋₁₀ alkyl, substituted or unsubstituted C₁₋₁₀ heteroalkyl, substituted or unsubstituted C₆₋₃₀ aryl, or substituted or unsubstituted C₄₋₃₀ heteroaryl, any two R⁴ groups together optionally forming a ring, and an R⁴ group and L² together optionally forming a ring;
X independently represents a halogen atom; p2 each L¹, L², and L³ independently represents a single bond or a divalent linking group; and
a, b, c independently represent an integer greater than or equal to 1;
d is 1 or 2, provided when c is greater than 1, d is 1;
wherein the core is optionally further substituted.

10. A polymer, comprising a repeat unit formed from the monomer of claim 8.

11. A polymer, comprising the reaction product of (i) a compound of claim 1, and (ii) a first polymer comprising a first repeat unit comprising a group chosen from one or more of —OH or —C(O)OH, wherein the reaction product comprises an acetal or ketal linker formed by reaction between an enol ether moiety or a halomethyl ether moiety of the compound and the —OH or —C(O)OH group of the first repeat unit, thereby forming a modified first repeat unit.

12. The polymer of claim 11, wherein the modified first repeat unit comprises a moiety of formula (4): 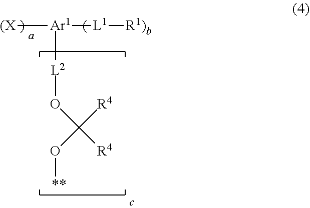
wherein:
Ar¹ is a C₆₋₃₀ aromatic or C₃₋₃₀ heteroaromatic core;
R¹ independently represents a moiety comprising an acid labile group;
R⁴ independently represents H, substituted or unsubstituted C₁₋₁₀ alkyl, substituted or unsubstituted C₁₋₁₀ heteroalkyl, substituted or unsubstituted C₆₋₃₀ aryl, or substituted or unsubstituted C₄₋₃₀ heteroaryl, any two R⁴ groups together optionally forming a ring, an R⁴ group and L² together optionally forming a ring, and an R⁴ group and another portion of the monomer not shown in formula (4) together optionally forming a ring;
X independently represents a halogen atom;
Each L¹ and L² independently represents a single bond or a divalent linking group;
a, b, and c independently represent an integer greater than or equal to 1; and
** represents an attachment point to another portion of the modified first repeat unit;
wherein the core is optionally further substituted.

13. The polymer of claim 11, wherein the first repeat unit of the first polymer is formed from a monomer comprising a polymerizable vinyl group.

14. The polymer of claim 10, further comprising a second repeat unit comprising an acid-labile group, a base-decomposable group, a aryl group, a fluoroalcohol group, or a lactone group, wherein the second repeat unit is structurally different from the modified first repeat unit.

15. A photoresist composition, comprising: a polymer of claim 10; and a solvent.

16. The photoresist composition of claim 15, further comprising a photoacid generator.

17. A pattern formation method, comprising:
(a) forming a photoresist layer from a photoresist composition of claim 15 on a substrate;
(b) exposing the photoresist layer to activating radiation; and
(c) developing the exposed photoresist layer to provide a resist relief image.
